(19) United States
(12) Patent Application Publication
(10) Pub. No.: US 2026/0285929 A1
(43) Pub. Date: Sep. 24, 2026

(54) RECOMBINANT AAV AS AN IMMUNE ADJUVANT

(71) Applicant: University of Florida Research Foundation, Incorporated, Gainesville, FL (US)

(72) Inventors: Duane Mitchell, Gainesville, FL (US); Todd Eliot Golde, Gainesville, FL (US); Christina von Roemeling, Gainesville, FL (US)

(73) Assignee: University of Florida Research Foundation, Incorporated, Gainesville, FL (US)

(21) Appl. No.: 19/128,746

(22) PCT Filed: Nov. 10, 2023
(86) PCT No.: PCT/US2023/079437

§ 371 Date: May 9, 2025

Related U.S. Application Data
(60) Provisional application No. 63/424,815, filed on Nov. 11, 2022.

(51) Int. Cl. C07K 14/52; A61K 38/00; A61K 39/00; A61K 48/00; A61P 35/00; C07K 16/28; C12N 15/86

(52) U.S. Cl. CPC C07K 14/522; A61K 48/0041; A61K 48/0058; A61P 35/00; C07K 16/2818; C12N 15/86; A61K 38/00; A61K 2039/505; C12N 2750/14143

(57) ABSTRACT

The present disclosure is related to improved compositions and methods related to viral immunotherapy treatments for cancer. Specifically, provided herein are therapeutic rAAV vectors, particles, and compositions thereof related to treatment of solid tumors. Provided herein are recombinant AAV particles that express a lymphocyte-recruiting chemokine, such as CXCL9, in tumor cells, such as glial cells of the brain, and thereby modify the tumor microenvironment to sensitize the tumor to other immune therapies. Kits, pharmaceutical compositions, and methods of treatment of brain tumors and other brain malignancies are contemplated herein. Combination therapies involving the administration of any of the disclosed rAAV particles and an immune modulator are also provided.

### RELATED APPLICATIONS

This application is a national stage filing under 35 U.S.C. 371 of International Patent Application Serial No. PCT/US2023/079437, filed Nov. 10, 2023, entitled “RECOMBINANT AAV AS AN IMMUNE ADJUVANT”, which claims the benefit of the filing date of U.S. Provisional Application No. 63/424,815, filed Nov. 11, 2022, entitled “RECOMBINANT AAV AS AN IMMUNE ADJUVANT”. The contents of each of these applications is incorporated herein by reference in their entirety.

### NON-FEDERAL SUPPORT

This work was supported, in whole or in part, by Circle of Hope for Cancer Research INC.

### REFERENCE TO AN ELECTRONIC SEQUENCE LISTING

The contents of the electronic sequence listing (U120270127US01-SEQ-AXW.xml; Size: 15,989 bytes; and Date of Creation: Mar. 18, 2025) is herein incorporated by reference in its entirety.

### BACKGROUND OF THE INVENTION

Glioma is a tumor of the glial cells in the brain and spinal cord. Three types of glial cells can produce tumors. The corresponding types of glioma are astrocytomas (tumors of astrocytes), which includes astrocytoma, anaplastic astrocytoma and glioblastoma multiforme (GBM); ependymoma; and oligodendroglioma. Gliomas are classified according to the type of glial cell involved in the tumor, as well as the tumor's genetic features, which can help predict how the tumor will behave over time and the treatments most likely to work. Inherited polymorphisms of DNA repair genes ERCC1, ERCC2 (XPD) and XRCC1 increase the risk of glioma. Surgical procedures are often the preferred initial treatments for gliomas. GBM is the most aggressive form of astrocytoma. In adults, GBM occurs most often in the cerebral hemispheres, especially in the frontal and temporal lobes of the brain. GBM is a devastating cancer that typically results in death in the first 15 months after diagnosis.

The current standard of care includes surgical resection, chemotherapy, and radiation, but does not result in remission for the majority of patients. Recently, gene therapy and immunotherapy approaches for treating glioma and glioblastoma have been developed. Although immunotherapy holds promise for producing durable outcomes in GBM, to date few patients have demonstrated a significant survival benefit despite numerous clinical trials examining different modalities to invigorate immune recognition and tumor eradication. And gene therapy treatments that have reached clinical trials for GBM have failed. In particular, viral vector-based treatments have not achieved FDA approval due to issues with viral delivery, inefficient tumor penetration, and limited efficacy. There remains a need in the art for viral vector-based constructs for gene delivery to specific cell types, such as tumor cells.

AAV has been shown to infect a variety of cell and tissue types, and significant progress has been made over the last decade to adapt this viral system for use in human gene therapy. Recombinant adeno-associated virus (rAAV) vectors have been used successfully for in vivo gene transfer in numerous pre-clinical animal models of human disease and have been used successfully for long-term expression of a wide variety of therapeutic genes. AAV vectors have also generated long-term clinical benefit in humans when targeted to immune-privileged sites. A major advantage of this vector is its comparatively low immune profile, eliciting only limited inflammatory responses and, in some cases, even directing immune tolerance to transgene products.

It is well-known that gliomas are devoid of lymphocytes, indicating that a major obstacle for effective immunotherapy is the limited ability of cytotoxic T lymphocytes (CTLs) to home to the tumor. CTLs are recruited via long-range signaling mediated by the diffusion of chemokines present in inflammatory environments. During glioma formation, tumors manufacture immune-suppressive chemokines and cytokines that co-opt resident cells, resulting in the preferential recruitment of immune suppressor cells from the periphery. Therefore, there remains a need in the art for an rAAV vector that can express proinflammatory chemokines in situ that can successfully recruit CTLs to the site of the tumor. In this manner, the rAAV vector may be able to trigger a stronger immune response against the tumor and sensitize the tumor to more conventional tumor treatments, thereby acting as an immune adjuvant.

### SUMMARY OF THE INVENTION

The present disclosure provides recombinant AAV (rAAV) vectors and particles that express transgenes encoding lymphocyte-recruiting chemokines and cytokines. In some aspects, these rAAV vectors contain nucleic acids encoding chemokines that have C-X-C motif ligands, such as CXCL9, CXCL10, and CXCL16, in cells of the central nervous system (CNS) to improve disease outcomes in cancers that implicate the CNS. The rAAV vectors of the disclosure may be packaged into viral particles, wherein the viral particles may be of serotype 6. Pharmaceutical compositions containing these rAAV particles are provided. The present disclosure also provides methods of treatment of a subject, and methods of transducing glial and/or immune cells, by administering these vectors and particles, as well as uses of these vectors in the manufacture of medicaments for treatment. In some aspects, combination therapies comprising administering the disclosed rAAV particle compositions with an immune checkpoint inhibitor are provided. The present disclosure also provides kits and cells comprising any of the disclosed rAAV particles.

The present disclosure is based, at least in part, on the discovery that viral vector-mediated delivery to the CNS of chemotactic factors that favor lymphocyte recruitment exhibit strong recruitment of cytotoxic T cells (CTLs) to solid tumors in the CNS of mammals. This recruitment triggers an immune response against the tumor that leads to reduction in size of the tumor and positive disease outcomes. These outcomes include greater survival of the mammalian subject. This discovery has led to the identification of viral vectors, such as rAAV vectors that express lymphocyte-recruiting chemokines and cytokines to achieve amelioration and treatment of the tumor. The cytokines and chemokines identified in the disclosed studies include CXCL9 (also known as MIG), MIP-1α/β (CCL3/CCL4), and other chemokines having a C—X—C motif. Such lymphocyte-recruiting chemokines and cytokines may be referred to herein as “call-and-receive” signal chemokines and CTL-recruiting chemokines. In some aspects, the recruited CTLs are CD8-expressing T cells (CD8+ CTLs).

The disclosure provides rAAV gene therapies for delivery of chemokine transgenes by a controlled route of delivery (e.g., intratumor, intracranial or intraventricular routes of delivery) to cells of interest in the CNS. These cells of interest include tumor-reactive astrocytes, tumor-associated macrophages, and tumor-associated microglia. These methods are designed to circumvent or overcome the limitations of systemic treatments—which include systemic toxicity, limited CNS permeability due to the presence of the blood brain barrier (BBB), accumulation in healthy CNS tissue. The disclosed rAAV compositions and methods provide increased efficacy with decreased systemic toxicity and do not require auxiliary methods to improve BBB permeability such as focused ultrasound. In some aspects, strategies to selectively reprogram the tumor microenvironment (TME) were explored using focal delivery of rAAV encoding call-and-receive chemokines for CTLs, such as CXCL9.

Thus, the excellent safety profile of AAV was combined with focal delivery, enhancing exposure within the tumor and mitigating systemic toxicities seen with conventional therapies. AAV therapy was successful at stimulating biological response to the encoded transgene, which increased lymphocyte recruitment. This strategy may be used across immunotherapy platforms. In some embodiments, the disclosed rAAV therapies may be used to treat any solid or hematologic primary CNS malignancy, and any secondary metastasis to the CNS. In particular embodiments, the disclosed rAAV therapies may be used to treat brain tumors such as glioma and GBM. In some embodiments, the disclosed rAAV particles exhibit tropism for and/or are adapted to transduce, astrocytes, microglia, and/or infilitrating myeloid cells. In exemplary embodiments, the disclosed rAAV particles exhibit tropism for astrocytes. Strong astrocyte transduction is correlated with high CXCL9 expression and CTL homing to the tumor, which results in modification of the TME.

Recombinant AAV transduction of the GBM TME to express CXCL9 (AAV6-CXCL9) enhances CTL tumor infiltration and improves responsiveness to immunotherapy. The disclosed rAAV particles, compositions and methods may encode any suitable CTL recruitment signal for any immune cell of interest. As such, rAAV particles encoding for a lymphocyte-recruiting chemokine or cytokine tailored to accommodate different tumors and TMEs are contemplated.

In the disclosed experiments, proteomic arrays were used to identify CTL chemokines absent in human glioma. 3D immunohistochemistry and flow cytometry were used to evaluate geospatial rAAV transgene expression and lymphocyte trafficking in vitro and in vivo. Survival studies of rAAV6-CXCL9 alone and in combination with anti-PD-1 checkpoint blockade were performed in preclinical models of GBM. Tumor immunogenicity following treatment was evaluated by single cell RNA sequencing (scRNAseq) and proteomic analysis. Several capsids were identified as having tropism for glial cells. In particular, AAV6 was identified as a reliable capsid for transducing murine tumor-reactive astrocytes. Transgene expression following a single intra-tumor injection was focal, stable, and resulted in high levels of CXCL9 production. CXCL9 was effective at promoting tumor T-cell chemotaxis in vitro and in vivo. AAV6-CXCL9 treatment sensitized GBM to PD-1 blockade, improving survival in two distinct syngeneic preclinical models. This sensitization effect that was in part dependent on recruitment of CD8 T-cells to the site of the tumor.

Accordingly, in some aspects, provided herein are recombinant adeno-associated virus (rAAV) particles comprising a vector containing an expression cassette comprising a transgene encoding a lymphocyte-recruiting chemokine. The chemokine may be any chemotactic factor that favors recruitment of lymphocytes such as CTLs. In some embodiments, the chemokine is selected from CXCL9, MIP-1a/B, CXCL10, CXCL16, and variants thereof. In some embodiments, the chemokine is CXCL9. In some embodiments, the chemokine is MIP-1a/B. In some embodiments, the chemokine-encoding transgene is a variant of CXCL9 or MIP-1a/B having 1, 2, 3, 4, 5, 6, 7, 8, 9, 10, 10-15, or 15 or more nucleotides that differ from the wild-type CXLC9 or MIP-1a/B protein sequence.

In various embodiments, the transgene is operably linked to a promoter. In various embodiments, the expression cassette is flanked on each side by an inverted terminal repeat sequence (ITR). The ITRs of the vector may be of any AAV serotype, such as serotype 2. The rAAV particle comprises a capsid protein. In some embodiments, the capsid protein is of serotype 6. In some embodiments, the capsid protein is a variant of serotype 6. In particular embodiments, the AAV particle is a pseudotyped rAAV2/6 particle.

The transgene of any of the disclosed rAAV vectors may be operably linked to one or more regulatory elements, such as a promoter. The promoter may be heterologous to the transgene. The promoter may be active in mediating expression in human cells or glial cells. The promoter may be a neuron-specific promoter. Exemplary such promoters include Glial Fibrillary Acidic protein (GFAP) promoter, MECP2 promoter, IBA1 promoter, JeT promoter, synapsin (SYN) promoter (e.g., human synapsin, or hSYN), and Microtubule-associated protein 2 (MAP2) promoter. Alternatively, the promoter may be non-cell autonomous and constitutively active, such as chicken beta-actin (CBA) promoter. The transgene may also be operably linked to a polyadenylation signal, such as a minimal polyA signal.

In some emobdiments, the rAAV vector is an rAAV2/6-GFAP-CXCL9 vector. In some emobdiments, the rAAV vector is an rAAV2/6-CBA-CXCL9 vector. In some emobdiments, the rAAV vector is an rAAV2/6-GFAP-MIP-1a/B vector. In some embodiments, any of these vectors are single-stranded.

In various embodiments, rAAV transduction in the CNS tissue or cell of interest is focal to the tumor and durable. In some embodiments, administration of any of the disclosed rAAV particles to a subject or a tissue results in increased expression of the encoded chemokine in the tissue for at least 5 days, 7 days, 14 days, 19 days, 21 days, or more than 21 days, relative to control or no treatment.

In some aspects, provided herein are compositions comprising a plurality of the disclosed rAAV particles. These compositions may further comprise a pharmaceutically acceptable carrier or excipient. They may be adapated for intracranial, intracerebroventricular (ICV), intrathecal, or intravenous (IV) delivery.

In some aspects, methods of treating a subject suffering from a cancer comprising administering a therapeutically effective amount of any of the disclosed rAAV particles or compositions are provided. The cancer may be any solid or hematologic primary CNS malignancy, and any secondary metastasis to the CNS. In particular embodiments, the cancer is a brain tumor. In some embodiments, methods of treatment of glioma and GBM are provided. In some embodiments, the disclosed rAAV compositions are used as a first line of therapy, or a second line of therapy. In some embodiments, the disclosed compositions are administered in conjunction or in combination with another therapy, such as an immune modulator.

Methods of treatment comprising administering any of the disclosed rAAV particles, or compositions thereof, and further administering an immune modulator are provided. Exemplary immune modulators include immune checkpoint inhibitors. As provided in the Examples, a combination therapy with anti-PD-1 blockade resulted in improved survival in evaluated mice subjects. This strategy is demonstrated across groups of immune checkpoint inhibitors that include PD-1, PD-L1, and CTLA-4 antagonists. In exemplary embodiments, an anti-PD-1 antibody, such as an FDA-approved monoclonal antibody, is administered in combination with the disclosed compositions. In some embodiments, one or more FDA-approved immune checkpoint inhibitors are administered before, after, or concurrently with the rAAV particle composition.

Administration of any of the disclosed compositions or combination therapies may result in strong recruitment of CD8 CTLs to the site of the tumor. Administration may result in reduction in size of the tumor and remission of the cancer. It may result in increased survival of the subject. Administration of any of the disclosed combination therapies may confer long-term innate and/or adaptive immunity against recurrence of the tumor. As such, uses of any of the disclosed rAAV particle to stimulate innate and adaptive immune responses in a subject are contemplated.

In some aspects, kits containing a composition of rAAV particles and a composition comprising an immune modulator are provided. In these kits, the rAAV particle composition and immune modulator composition may be provided in separate containers. Host cells comprising any of the disclosed rAAV particles are also provided. These host cells may be human glial cells.

In some aspects, the disclosure provides methods of treatment of a subject having a cancer comprising administering to the subject any of the rAAV particles or vectors encoding any of the transgenes described herein. In some aspects, the subject is a human. In some embodiments, the disclosed rAAV particles, pharmaceutical compositions, or cells are administered through an intravenous, intracranial, intrathecal, or intracerebroventricular infusion. In some embodiments, disclosed rAAV particles, pharmaceutical compositions, or cells are administered directly to a tumor of the subject (such as a brain tumor).

### BRIEF DESCRIPTION OF THE DRAWINGS

The following drawings form part of the present specification and are included to demonstrate certain aspects of the present invention. The invention may be better understood by reference to the following description taken in conjunction with the accompanying drawings, in which like reference numerals identify like elements, and in which:

FIGS. 1A-1F show the chemokine signature of glioblastoma tumors. FIG. 1A shows the reference IHC of CD3D expression in human GBM tumor tissue depicting undetectable and low staining frequencies (images from proteinatlas.org/ENSG00000167286-CD3D/pathology/glioma). FIG. 1B shows the distribution of CD3D expression in human GBM, sorted into undetected, low (<25% positive cells), moderate (25-75% positive cells), or high (>75% positive cells) expression levels, based on the cumulative results of two independent antibody stains where low levels represent the highest degree of signal detected among all specimens examined. FIG. 1C shows immunoblots of n=8 independent GBM samples showing signal intensity of 31 chemokines. CXCL9, undetected, is outlined. FIG. 1D shows the cumulative relative protein expression levels of immunoblots shown in FIG. 1C. FIG. 1E is a schematic depicting the transduction of the tumor and/or TME with AAV encoding CXCL9 following direct intra-tumor delivery produces a call-and-receive signal for lymphocytes. This resulted in increased lymphocyte trafficking into the tumor through chemokine-receptor engagement between CXCL9 in the TME and CXCR3 expression on lymphocytes. FIG. 1F depicts the recombinant AAV6 vector design encoding CXCL9 and the fluorescent reporter gene RFP separated by an internal ribosome entry sequence (IRES).

FIGS. 2A-2I show AAV6 demonstrates tropism for tumor-reactive astrocytes. FIG. 2A is a 3D IHC of RFP-labeled GL261 tumor tissue collected 1 week following AAV6-EGFP injection, n=3. Top row represents 3D rendering of tissue, where AAV6 transduced cells are shown in green pseudocolor, GFAP in red pseudocolor, RFP+ tumor cells in light blue pseudocolor, and DAPI nuclear stain in dark blue pseudocolor. Second row depicts 2D visual rendering of co-localization detected between AAV6 transduced cells (EGFP+) and GFAP+ cells, where voxel overlap (co-localization) is shown as a separate channel (yellow pseudocolor). Similarly, the third row shows co-localization between AAV6 transduced cells and tumor cells, with voxel overlap between tumor and EGFP shown as a separate channel (pink pseudocolor). FIG. 2B shows quantitative analysis depicting the proportion of EGFP (AAV6) co-localization with tumor cells, astrocytes, or undefined cells in the GL261 tumor model, n=3 per group. Values are presented as the cumulative mean±standard deviation. FIG. 2C is imaging and co-localization as described in panel a using the KR158 GBM model. FIG. 2D shows quantitative analysis of EGFP (AAV6) co-localization with tumor cells, astrocytes, or undefined cells in the KR158 tumor model, n=3 per group. Values are presented as the cumulative mean±standard deviation. FIG. 2E is a 3D IHC of AAV6-EGFP transduction in age-matched naïve control mice (n=3). Top row represents 3D rendering of tissue, where AAV6 transduced cells are shown in green pseudocolor, GFAP in red pseudocolor, and DAPI nuclear stain in dark blue pseudocolor. Second row depicts 2D visual rendering of co-localization detected between AAV6 transduced cells (EGFP+) and GFAP+ cells, where voxel overlap (co-localization) is shown as a separate channel (yellow pseudocolor). FIG. 2F shows quantitative analysis depicting the proportion of EGFP (AAV6) co-localization with astrocytes or undefined cells in naïve mice, n=3. Values are presented as the cumulative mean±standard deviation. FIG. 2G shows intra-tumor AAV6 treatment schematic for early time point tissue collection and flow cytometry analysis. FIG. 2H are box-whisker plot with individual values shown depicting the percent of AAV6-EGFP transduced cells identified by flow cytometry as either RFP+GL261 tumor cells or GFAP (APC+) astrocytes at 3, 5, and 7 days following intratumor AAV6 injection (n=6 per time point). Two-way ordinary ANOVA statistical analysis performed comparing percent transduction between tumor and astrocytes across all time points. FIG. 2I shows representative flow plots for each time point in FIG. 2G, identifying each tumor and astrocyte populations where AAV6 transduced tumors are identified as EGFP⁺RFP⁺, and AAV6 transduced astrocytes are identified as EGFP⁺RFP⁻GFAP⁺. P-values=or <0.05 are considered statistically significant.

FIGS. 3A-3F show AAV6 transduction efficiency in GBM tumors. FIGS. 3A-3B are 3D IHC displaying geospatial distribution of AAV6 encoded transgene (BFP) in GL621 (FIG. 3A) and KR158 (FIG. 3B) tumors collected 1 week following AAV6-BFP injection (green pseudocolor), n=2 per model. DRAQ5 nuclear dye (pink pseudocolor) is used to identify tumor borders, as outlined by the white dashed line. FIG. 3C depicts the intra-tumor AAV6 treatment schematic for protein detection of AAV6 encoded CXCL9. FIG. 3D shows ELISA detection of CXCL9 protein in serum samples extracted from peripheral blood draws at one and two weeks following AAV6-CXCL9 or AAV6-EGFP control intracranial injection in GL261 (FIG. 3D) models, n=3 per time point, per group. Serum protein levels of CXCL9 isolated from age-matched naïve controls used to establish baseline levels indicated by dashed black line. FIGS. 3E-3F show ELISA detection of CXCL9 protein in brain tissues isolated at 1 and 2 weeks following AAV6-CXCL9 or AAV6-EGFP control intracranial injection in GL261 (FIG. 3E) and KR158 (FIG. 3F) models. Cerebellar tissue was removed, and left and right hemispheres lysed separately to reflect tumor-bearing and contralateral (focal and distal) signal detection. Statistical analyses performed using two-way ANOVA analysis with Tukey's multiple comparisons test. Naïve brain, and sham (saline) injected tumors included as negative control and tumor baseline control, with the latter represented by the dashed black line, n=3-4 per group. N=3-6 for rAAV6 injected tumor groups, with individual values shown. P-values=or <0.05 are considered statistically significant.

FIGS. 4A-4J show AAV6-CXCL9 directed lymphocyte chemotaxis. FIG. 4A depicts a diagrammatic overview of in vitro competitive T lymphocyte chemotaxis assay. FIG. 4B is a competitive chemotaxis measured as the number of T lymphocytes (CTV+, blue pseudocolor) in either AAV6-EGFP (control, green pseudocolor) transduced GL261 field or AAV6-CXCL9 (RFP+, red pseudocolor) transduced GL261 field at 1 and 24 hours following co-culture. Statistical analyses performed by two-way ANOVA with Sidak's multiple comparisons test, n=3 per time point, per group. Representative images of competitive chemotaxis shown. Dashed white line represents the lymphocyte-tumor border at assay start. FIG. 4C is a competitive chemotaxis measured as the number of T lymphocytes (CTV+, blue pseudocolor) in either AAV6-EGFP (control, green pseudocolor) transduced or AAV6-CXCL9 (RFP+, red pseudocolor) transduced C8-D1A astrocytes field at 1 and 24 hours following co-culture. Statistical analyses performed by two-way ANOVA with Sidak's multiple comparisons test, n=3 per time point, per group. Representative images of competitive chemotaxis shown. Dashed white line represents the lymphocyte-astrocyte border at assay start. FIG. 4E is a multicolor flow cytometric detection of tumor-infiltrating CD8+ lymphocyte subsets in single or combination AAV6-CXCL9 plus anti-PD-1 immune checkpoint blockade treatment in GL261 model. AAV6-EGFP and IgG2 mAb control treatment included. Fold-change normalization based on values detected in sham (saline) injected tumor control samples, with mean expression indicated by dashed black line. Statistical analyses performed using ordinary one-way ANOVA with Tukey's multiple comparisons test, n=5-6 per treatment group, individual values shown. FIG. 4F is a multicolor flow cytometric detection of tumor-infiltrating CD4+ lymphocyte subsets in single or combination AAV6-CXCL9 plus anti-PD-1 immune checkpoint blockade treatment in GL261 model. AAV6-EGFP and IgG2 mAb control treatment included. Fold-change normalization based on values detected in sham (saline) injected tumor control samples, with mean expression indicated by dashed black line. Statistical analyses performed using ordinary one-way ANOVA with Tukey's multiple comparisons test, n=5-6 per treatment group, individual values shown. FIG. 4G show concatenated flow plots for all treatment groups represented in FIGS. 4E and 4F. FIGS. 4H-4I show a biological repeat of experiments presented in FIGS. 4E, 4F, and 4G using the KR158 tumor model. P-values=or <0.05 are considered statistically significant.

FIGS. 5A-5F show the scRNAseq data.

FIGS. 6A-6E show the scRNAseq data.

FIGS. 7A-7G show Combination AAV6-CXCL9 plus anti-PD-1 immunotherapy reprograms immunological landscape of GBM. FIG. 7A shows representative cytokine immunoblots from all treatment groups. FIG. 7B is a heatmap shown denoting differentially expressed inflammatory cytokines detected single agent and/or combination AAV6-CXCL9 plus anti-PD-1 checkpoint inhibitor treated tumor tissue as compared to sham treated control tumors, n=3-4 per group. Violin plots of relative protein expression of (FIG. 7C) CCL5, (FIG. 7D) CD40, and (FIG. 7E) CXCL10 detected in tumors across all treatment groups. Statistical analyses performed using Kruskal-Wallis multiple comparisons test, n=3-4 per group, individual values shown. FIG. 7F shows circos interactome analysis (in progress). FIG. 7G shows GREAT images & quantitation (in progress). P-values=or <0.05 are considered statistically significant.

FIGS. 8A-8H show AAV6-CXCL9 sensitizes GBM tumors to anti-PD-1 immunotherapy, improving overall survival. FIG. 8A depicts a diagrammatic summary of combinatorial treatment strategy. Survival analysis in (FIG. 8B) GL261 and (FIG. 8C) KR158 tumor-bearing mice treated with sham (saline) control, AAV6-CXCL9, and anti-PD-1 monoclonal antibody alone and in combination. AAV6-EGFP and IgG2 included as treatment controls. Median survival for each treatment group shown, n=8 per group. Statistical analysis performed using Log-rank (Mantel-Cox) test comparing individual treatment groups. FIG. 8D depicts a diagrammatic summary of combination treatment strategy with concomitant CD8a antibody depletion. FIGS. 8E and 8F show flow cytometry detection of lymphocyte subsets isolated from peripheral blood collected at day 18 of study. Bar graph (8E) depicts the percent of each CD4 T lymphocytes (CD45+CD3+CD4+CD8−) and CD8 T lymphocytes (CD45+CD3+CD4−CD8+) detected within the total CD45+ population of PBMCs. Statistical analyses performed using Sidak's multiple comparisons test, n=5-8 per group. Individual values shown. FIG. 8G shows the survival analysis in GL261 tumor-bearing mice treated with combination AAV6-CXCL9 plus anti-PD-1 monoclonal antibody, with or without anti-CD8a depletion. Sham (saline) injected GL261 tumors treated with anti-CD8a or control IgG2 included as control groups. Median survival for each treatment group shown, n=8 per group. Statistical analysis performed using Log-rank (Mantel-Cox) test comparing individual treatment groups. P-values=or <0.05 are considered statistically significant. FIG. 8H shows survival on rechallenge.

FIG. 9 shows a quantitative summary of voxel-based AAV6 co-localization with either tumor (GL261, n=5) or astrocytes in each tumor-bearing (n=3) and naïve mice (n=3). Values are presented as the cumulative mean±standard deviation.

FIG. 10 shows KR158 models, n=3 per time point, per group. Age-matched naïve controls used to establish baseline CXCL9 levels indicated by dashed black line. ELISA detection of CXCL9 protein in brain tissues isolated at 1 and 2 weeks following AAV6-CXCL9 or AAV6-EGFP control intracranial injection in (FIG. 3E) GL261 and (FIG. 3F) KR158 models.

FIGS. 11A-11E shows (FIG. 11A) a schematic outlining combination AAV6 and PD-1 ICB treatment and tissue collection and survival analysis in preclinical models. Multicolor flow cytometric detection of tumor-infiltrating CD8+ lymphocyte subsets in single or combination AAV6-CXCL9 plus anti-PD-1 ICB treatment in (FIG. 11B) GL261 and (FIG. 11C) KR158 models. AAV6-EGFP and IgG2 mAb control included as treatment controls. Fold-change normalization based on values detected in sham (saline) injected tumor control samples, with mean expression indicated by dashed black line. Statistical analysis performed using ordinary one-way ANOVA with Fisher's least significant difference (LSD) test for multiple comparisons, n=3-6 per treatment group, individual values shown. Multicolor flow cytometric detection of tumor-infiltrating CD4+ lymphocyte subsets in single or combination AAV6-CXCL9 plus anti-PD-1 immune checkpoint blockade treatment in (FIG. 11D) GL261 and (FIG. 11E) KR158 models. AAV6-EGFP and IgG2 mAb treatment controls included. Fold-change normalization based on values detected in sham (saline) injected tumor control samples, with mean expression indicated by dashed black line. Statistical analysis performed using ordinary one-way ANOVA with Fisher's LSD test for multiple comparisons, n=3-6 per treatment group, individual values shown. P-values=or <0.05 are considered statistically significant.

FIGS. 12A-12B show tile-stitch 10× 3D IF imaging of GL261 tumors resected from combination AAV6-CXCL9 plus anti-PD-1 ICB treated GREAT mice (FIG. 12A). DAPI nuclear dye (blue pseudocolor) used to identify tumor area outlined by the dashed white line. Digital magnification of regions outlined in the far-left panel (a′ and b′) are included to show higher image resolution. Green pseudocolor depicts endogenous EYFP, correlating with IFNγ expression. (FIG. 12B) 3D IF of tissue from (FIG. 12A) immunolabeled for CD45 expression (red pseudocolor). Digital zoom of region outlined in the far-right panel shows co-localization between CD45 and IFNγ, indicating these are immune cells.

FIGS. 13A-13J show the immunological landscape of GBM tumors treated with AAV6-CXCL9 and anti-PD-1 immunotherapy. UMAP of cell types clustered by scRNA transcriptional analysis of 52,344 CD45+ cells isolated from GL261 tumor bearing mice treated with: (FIG. 13A) sham (saline), (FIG. 13B) AAV6-ctrl+IgG2, (FIG. 13C) AAV6-ctrl+aPD-1, (FIG. 13D) AAV6-CXCL9+IgG2, and (FIG. 13E) combination AAV6-CXCL9+aPD-1 treated GL261 tumors, n=3 per group. Summary circle chart depicting cell cluster population frequency detected for each treatment included alongside each UMAP. FIG. 13F shows a summary of UMAP cell clusters. Quantitative change in population frequency of (FIG. 13G) CD8+ T cells, (FIG. 13H) Treg cells, (FIG. 13I) monocytes, and (FIG. 13J) non-classical (n-c) monocytes across treatment groups. Statistical analyses performed using ordinary one-way ANOVA with Fisher's LSD test for multiple comparisons, n=3 per group, individual values shown.

FIGS. 14A-14L show AAV6-CXCL9 and anti-PD-1 immunotherapy stimulates CD8 lymphocyte activation. FIG. 14A shows a Venn Diagram representing differentially expressed genes affiliated with each treatment. FIG. 14B shows a heatmap depicting scRNA-seq-derived cell-cell communication networks enriched or decreased in response to combination AAV6-CXCL9+aPD-1 as compared to AAV6-CXCL9+IgG2 treatment across identified cell clusters. FIG. 14C shows a heatmap depicting scRNA-seq-derived cell-cell communication networks enriched or decreased in response to combination AAV6-CXCL9+aPD-1 as compared to AAV6-EGFP+aPD-1 treatment across identified cell clusters. FIG. 14D shows a waterfall summary plot of scRNA-seq-derived signaling pathways enriched in CD8+ T cells following combination AAV6-CXCL9+aPD-1 as compared to AAV6-CXCL9+IgG2 treatment. FIG. 14E shows a waterfall summary plot of scRNA-seq-derived signaling pathways enriched in CD8+ T cells following combination AAV6-CXCL9+aPD-1 as compared to AAV6-EGFP+aPD-1 treatment. FIG. 14F shows a heatmap representation of gene expression analysis derived from all cell clusters using the nCounter® Immune Exhaustion Panel (nanoString) following AAV6-CXCL9 gene therapy with or without PD-1 ICB. CD8+ T cell populations outlined in black for each treatment group. FIGS. 14G-14L show he quantification of common pathways found to be differentially regulated in CD8+ T cells in response to treatment. Statistical analyses performed using Kruskal-Wallis test followed by Dunn's multiple comparisons, with individual values shown. P-values=or <0.05 are considered statistically significant.

FIGS. 15A-15K show the inflammatory signature of preclinical GBM treated with AAV6-CXCL9 and anti-PD-1 ICB. FIG. 15A shows a heatmap summary of scRNA-seq-derived CCL-CXC expression in CD8+ T cells in response to AAV6-CXCL9 and anti-PD-1 ICB treatment created using GraphPad Prism. FIGS. 15B-15I show the quantification of CCL-CXC genes found to be differentially expressed in CD8+ T cells in response to treatment. Statistical analyses performed using Kruskal-Wallis test followed by Dunn's multiple comparisons, with individual values shown. FIG. 15J shows a heatmap summary of CCL-CXC relative protein expression found to be differentially expressed in response to AAV6-CXCL9 with and without PD-1 ICB, created using GraphPad Prism. FIG. 15K shows the Circos interactome analysis of detected differentially expressed proteins and predicted receptors. P-values=or <0.05 are considered statistically significant.

FIG. 16 shows a diagrammatic summary of findings. Intra-tumor delivery of AAV6 encoding CXCL9 results in robust transduction of tumor-reactive astrocytes, creating a chemotactic gradient of secreted CXCL9. This improves lymphocyte trafficking in combination with anti-PD-1 ICB through chemokine-receptor engagement between CXCL9 in the TME and CXCR3 expression on lymphocytes. CD8+ T cells are required for durable survival response to treatment, indicating that tumor cell killing is mediated by the adaptive arm of immunity. Combination treatment also transforms the inflammatory milieu of tumors, creating a pro-inflammatory environment evidenced by the presence of cytokines and chemokines that further promote innate and adaptive immune activation.

FIGS. 17A-17F show flow cytometry results. FIG. 17A shows a heatmap summary of EGFP fluorescence intensity detected in vitro in 15 primary human and murine glioma cell lines 72 hours following transduction with 29 unique AAV serotypes. FIG. 17B AAV6-EGFP control vector map. FIG. 17C shows AAV6-BFP control vector map. FIG. 17D shows a AAV6-empty vector control map. FIGS. 173-17F showin vitro transduction efficiency of AAV6-EGFP detected via quantitative flow cytometry in GL261, KR158, and CT-2A murine glioma 72 hours post-transduction (1e5 VGS), n=3 per model. Statistical analyses performed using two-way ANOVA with Sidak's multiple comparisons test. P-values=or <0.05 are considered statistically significant.

FIGS. 18A-18B show 3D immunofluorescence data of KR158 (FIG. 18A) and GL261 (FIG. 18B) tumors 1 week following AAV6-EGFP (green pseudocolor) intratumor injection (1e10 VGS) counter-labeled with DAPI nuclear stain (blue pseudocolor). Left panels show 3D rendering, and right panels depict select 2D images from Z-stack to show enhanced cellular resolution.

FIGS. 19A-19B show 3D immunofluorescence results. FIG. 19A shows results in KR158 tumors and FIG. 19B shows GL261 tumors implanted in CCR2RFPCX3CR1GFP reporter mice resected 1 week following intratumor injection with AAV6-BFP (1e10 VGS) and counter-labeled with DRAQ5 nuclear stain. Left panels show 3D rendering, and right panels depict select 2D images from Z-stack to show enhanced cellular resolution. Digital zoom of selected regions outlined by yellow dashed line to show lack of overlap between BFP and either RFP (CCR2) and GFP (CX3CR1).

FIGS. 20A-20B show gating strategies for flow cytometry. FIG. 20A shows a schematic illustration of the flow cytometry gating strategy and controls used in FIG. 2G. FIG. 20B shows a schematic illustration of the flow cytometry gating strategy and controls used in FIGS. 11B-11E, and FIGS. 8E-8F.

FIGS. 21A-21H show quantitative change in population frequency of (FIG. 21A) CD4+ T cells, (FIG. 21B) microglia, (FIG. 21C) dendritic cells (cDCs), (FIG. 21D) disease-associated microglia, (FIG. 21E) macrophages, (FIG. 21F) suppressive macrophages, (FIG. 21G) natural killer (NK) cells, and (FIG. 21H) border-associated macrophages (BAM) across treatment groups detected by scRNA-seq. Statistical analyses performed using ordinary one-way ANOVA with Fisher's least significant difference (LSD) test for multiple comparisons, n=3 per group, individual values shown. P-values=or <0.05 are considered statistically significant.

FIGS. 22A-22H show a summary chord plots of ligand-receptor interactions between scRNA-seq defined cell clusters generated using CellChat33, where line thickness correlates with the total number of predicted interactions between two defined cell clusters in each (a) sham, (b) AAV6-EGFP+IgG2, (c) AAV6-EGFP+aPD1, (d) AAV6-CXCL9+IgG2, and (e) AAV6-CXCL9+aPD1. (f) Waterfall summary plot of scRNA-seq-derived signaling pathways enriched in CD4+ T cells following combination AAV6-CXCL9+aPD-1 as compared to AAV6-CXCL9+IgG2 treatment. (g) Waterfall summary plot of scRNA-seq-derived signaling pathways enriched in CD4+ T cells following combination AAV6-CXCL9+aPD-1 as compared to AAV6-EGFP+aPD-1 treatment. (h) Box-whisker summary of pathway enrichment analysis for immune cells performed with AUCell algorithm using the nanoString nCounter® Immune Exhaustion pathway dataset. Statistical analysis performed using Kruskal-Wallis and Dunn's multiple comparison tests. P-values=or <0.05 are considered statistically significant.

FIGS. 23A-23M show graphs of gene expression in monocytes. FIG. 23A shows a heatmap representation of gene expression analysis derived from monocytes using the nCounter® Immune Exhaustion Panel (nanoString) following AAV6-CXCL9 gene therapy with or without PD-1 ICB. FIGS. 23B-23M show the quantification of common pathways differentially expressed in monocytes in response to treatment. Statistical analyses performed using Kruskal-Wallis test followed by Dunn's multiple comparisons, with individual values shown. P-values=or <0.05 are considered statistically significant.

FIGS. 24A-24H show graphs of protein expression. FIG. 24A shows a heatmap of relative protein expression of 65 out of 111 total inflammatory cytokines detected in sham control, single agent, and combination AAV6-CXCL9 plus anti-PD-1 checkpoint inhibitor treated tumor tissue collected 10 days after the onset of treatment, n=3-4 per group. Undetected proteins not displayed. FIGS. 24B-24H show violin plots of relative protein expression of differentially expressed chemokines and cytokines detected in tumors in response to AAV6-CXCL9 with or without anti-PD-1 ICB. Statistical analyses performed using Kruskal-Wallis multiple comparisons test, n=3-4 per group, individual values shown. P-values=or <0.05 are considered statistically significant.

### DETAILED DESCRIPTION

The present disclosure is related to improved compositions and methods related to viral immunotherapy treatments for cancer. Specifically, provided herein are therapeutic rAAV vectors and methods related to treatment of solid tumors. Provided herein are recombinant AAV particles that express a lymphocyte-recruiting chemokine in tumor cells, such as glial cells of the brain, and thereby modify the tumor microenvironment to sensitize the tumor to other immune therapies. Kits, pharmaceutical compositions, and methods of treatment of brain tumors and other brain malignancies are contemplated herein. Combination therapies involving the administration of any of the disclosed rAAV particles and an immune modulator—such as an immune checkpoint inhibitor or adoptive cell immunotherapy—are provided.

The present disclosure further provides methods of transducing expression of CXCL9 or MIP-1a/B in one or more brain cells comprising administering to the cells any of the disclosed rAAV vectors. These methods may confer enhanced biodistribution of the CXCL9 protein in the cells as well as enhanced transduction. The present disclosure also provides host cells comprising these rAAV-CXCL9 vectors and particles and compositions comprising these rAAV-CXCL9 vectors.

The disclosure also provides rAAV particles and compositions comprising rAAV particles having mutant capsid proteins. The disclosure provides methods and compositions for delivering rAAV particles having mutant capsid proteins to neural tissue, such as central nervous system (CNS) tissue. In particular embodiments, methods are provided for delivery to CNS tissue such as brain tissue (e.g., human brain tissue). The disclosure further provides methods for treatment of cancer in a subject suffering from or diagnosed with glioblastoma or glioma. The disclosure further provides methods of treatment of a subject in need thereof (such as a human subject) by administering one or more of these rAAV particles or compositions. In some embodiments, these methods comprise delivering a heterologous gene or targeting construct to a bain cell (such as an astrocyte or a glioma cell) comprising inserting the heterologous gene or targeting contruct into any of the disclosed AAV particles, and contacting the glioma cell with the AAV. The step of contacting the cell may comprise a single AAV particle administration or multiple AAV administrations (such as a regimen of multiple administrations). In some embodiments, any of the disclosed methods of treatment provide a reduction, either partial or complete, of a glioma tumor. In some embodiments, the disclosed methods of treatment induce apoptosis or cell death in a glioma.

Provided herein are methods of transducing cells and tissue in the tumor microenvironment. These cells may comprise astrocytes, microglia, monocytes, or other glial cells. These cells may comprise malignant or non-malignant cells. In some embodiments, methods of transducing astrocytes are provided. In some embodiments, methods of transducing these cells comprise providing to the cell any one of the compositions of variant rAAV particles as disclosed herein, wherein the rAAV particles in the composition comprise an expression cassette comprising a transgene (e.g., CXCL9). The disclosure also provides host cells (such as mammalian cells) comprising any of these particles or compositions. Further provided herein are methods of identifying AAV variants having increased affinity for and transduction of specific tumor cell type.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

A “subject” refers to mammal that is the object of treatment using a method or composition as provided for herein. “Mammal” includes, without limitation, mice, rats, rabbits, guinea pigs, dogs, cats, sheep, goats, cows, horses, primates, such as monkeys, chimpanzees, and apes, and humans. In some embodiments, the subject is human.

The terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. To “treat” a disease as the term is used herein, means to reduce the frequency or severity of at least one sign or symptom of a disease or disorder experienced by a subject.

The term “therapeutically effective amount,” as used herein, refers to an amount that is capable of treating or ameliorating a disease or condition or otherwise capable of producing an intended therapeutic effect, such as reducing the frequency or severity of at least one sign or symptom of a disease or disorder experienced by a subject.

A “nucleic acid” sequence refers to a deoxyribonucleic acid (DNA) or ribonucleic acid (RNA) sequence. This term encompasses naturally-occurring and non-naturally occurring nucleobases (bases). This term encompasses sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “polynucleotide,” refers to a polymeric form of nucleotides of any length, including DNA, RNA, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

For the purpose of describing the relative position of nucleotide sequences in a particular nucleic acid molecule throughout the instant application, such as when a particular nucleotide sequence is described as being situated “upstream,” “downstream,” “3′,” or “5” relative to another sequence, it is to be understood that it is the position of the sequences in the “sense” or “coding” strand of a DNA molecule that is being referred to as is conventional in the art.

The term “isolated” when referring to a nucleotide sequence, means that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not materially affect the basic characteristics of the composition.

As used herein, the term “variant” refers to a molecule having characteristics that deviate from what occurs in nature, e.g., a “variant” of a protein or nucleic adi molecule is at least about 80% identical, at least about 90% identical, at least about 95% identical, at least about 96% identical, at least about 97% identical, at least about 98% identical, at least about 99% identical, at least about 99.5% identical, or at least about 99.9% identical to the reference molecule (which may be a wild-type sequence). Variants of a protein molecule may contain modifications to the sequence (e.g., having 1, 2, 3, 4, 5, 6, 7, 8, 9, 10, 10-15, or 15-20 amino acid substitutions) relative to the wild-type sequence. Variants of a nucleic acid molecule may contain modifications to the sequence (e.g., having 1, 2, 3, 4, 5, 6, 7, 8, 9, 10, 10-15, or 15-20 nucleotide substitutions) relative to the wild-type sequence. These modifications include chemical modifications as well as truncations.

The term “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The “percent (%) identity” of two sequences, whether nucleic acid or amino acid sequences, is the number of exact matches between two aligned sequences divided by the length of the shorter sequences and multiplied by 100. This term refers to the extent to which two sequences (nucleotide or amino acid) have the same residue at the same positions in an alignment. For example, “an amino acid sequence is X % identical to SEQ ID NO: 6” refers to % identity of the amino acid sequence to SEQ ID NO: 6 and is elaborated as X % of residues in the amino acid sequence are identical to the residues of sequence disclosed in SEQ ID NO: 6. Generally, computer programs are employed for such calculations.

Sequence identity can be determined by aligning sequences using algorithms, such as BESTFIT, FASTA, FASTDB and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

For example, whether any particular nucleic acid molecule is at least 80%, 85%, 90%, 95%, 96%, 97%, 98%, or 99% identical to, for instance, the nucleotide sequence of a CXCL9 sequence, can be determined conventionally using known computer programs. A preferred method for determining the best overall match between a query sequence (e.g., a sequence of the present disclosure) and a subject sequence, also referred to as a global sequence alignment, can be determined using the FASTDB or blastn computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. Whether a nucleotide is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present disclosure. For subject sequences truncated at the 5′ and/or 3′ ends, relative to the query sequence, the percent identity is corrected by calculating the number of nucleotides of the query sequence that are positioned 5′ to or 3′ to the query sequence, which are not matched/aligned with a corresponding subject nucleotide, as a percent of the total bases of the query sequence.

The term “recombinant,” as applied to a polynucleotide means that the polynucleotide is the product of various combinations of cloning, restriction or ligation steps, and other procedures that result in a construct that is distinct from a polynucleotide found in nature and/or a combination of polynucleotides and viral proteins that is not found in nature. A recombinant virus is a viral particle comprising a recombinant polynucleotide. The terms respectively include replicates of the original polynucleotide construct and progeny of the original virus construct.

The term “gene,” refers to a polynucleotide containing at least one open reading frame that is capable of encoding a particular gene product. Any of the polynucleotide sequences described herein may be used to identify larger fragments or full-length coding sequences of the genes with which they are associated. Methods of isolating larger fragment sequences are known to those of skill in the art.

The term “transgene,” as used herein, refers to a nucleic acid sequence to be positioned within a viral vector and encoding a polypeptide, protein or other product of interest. In some embodiments, one rAAV vector may comprise a sequence encoding one or more transgenes (which can optionally be the same gene, or different genes). For example, one rAAV vector may comprise the coding sequence for 1, 2, 3, 4, 5, 6, 7, 8, 9, or 10 transgenes. The transgenes of the present disclosure are chemokines and cytokines and include, but are not limited to, CXCL9 and MIP-1α/β (CCL3/CCL4).

The terms “gene transfer” or “gene delivery” refer to methods or systems for inserting DNA, such as a transgene, into host cells, such as those of a subject afflicted with a cancer. In several embodiments, gene transfer yields transient expression of non-integrated transferred DNA, extrachromosomal replication and expression of transferred replicons (e.g., episomes). In additional embodiments, gene transfer results in integration of transferred genetic material into the genomic DNA of host cells. Exemplary host cells of the disclosure include HEK293 and C2C12 myoblast cells. Exemplary host cells of the disclosure include HEK293 and C2C12 myoblast cells. Additional exemplary host cells of the disclosure include human cells derived from an induced pluripotent stem cell or a neuronal cell line.

The terms “regulatory element” or “regulatory sequence”, or variations thereof, refer to a nucleotide sequence that participates in functional regulation of a polynucleotide, including replication, duplication, transcription, splicing, translation, or degradation of the polynucleotide. Regulatory elements can be enhancing or inhibitory in nature, depending on the embodiment. Non-limiting examples of regulatory elements include transcriptional regulatory sequences such as promoter sequences, polyadenylation (polyA) signals, transcription termination sequences, upstream regulatory domains, origins of replication, internal ribosome entry sites (“IRES”), enhancers, and the like. In some embodiments, any of the disclosed rAAV vectors comprise an IRES. In some embodiments, any of the disclosed rAAV vectors comprise a splice donor/splice acceptor (SD/SA) sequence, such as an endogenous SD/SA sequence. These elements collectively provide for the replication, transcription and translation of a coding sequence in a recipient cell, though not all of these sequences need always be present. It shall be appreciated that the structural components of a rAAV vector as provided for herein may be listed in individual paragraphs solely for clarity and may be used together in combination. For example, any regulatory element or other component can be used in combination with any transgene (or transgenes) provided for herein.

A “promoter” is a polynucleotide that interacts with an RNA polymerase and initiates transcription of a coding region (e.g., a transgene) usually located downstream (in the 3′ direction) from the promoter.

The term “operably linked” refers to an arrangement of elements wherein the components are configured to perform a function. For example, regulatory sequences operably linked to a coding sequence result in the expression of the coding sequence. Depending on the embodiment, a regulatory sequence need not be contiguous with the coding sequence. Thus, for example, one or more untranslated, yet transcribed, sequences can be present between a promoter sequence and a coding sequence, with those two sequence still being considered “operably linked”.

The term “vector” means any molecular vehicle, such as a plasmid, phage, transposon, cosmid, chromosome, virus, viral particle, virion, etc. which can transfer gene sequences (e.g., a transgene) to or between cells of interest.

An “expression vector” is a vector comprising a region of nucleic acid (e.g., a transgene) which encodes a gene product (e.g., a polypeptide or protein) of interest. As disclosed herein, vectors are used for achieving expression, e.g., stable expression, of a protein in an intended target cell (e.g., a cell of the nervous system). An expression vector may also comprise control elements operatively linked to the transgene to facilitate expression of the encoded protein in the target cell (e.g., a cell of the nervous system). A combination of one or more regulatory elements and a gene or genes to which they are operably linked for expression may be referred to herein as an “expression cassette.”

The term “AAV” is an abbreviation for adeno-associated virus, and the term may be used to refer to the virus itself or derivatives thereof. The term covers all subtypes and both naturally occurring and recombinant forms, unless otherwise indicated. The abbreviation “rAAV” refers to recombinant adeno-associated virus, also referred to as a recombinant AAV vector (or “rAAV vector”), which refers to AAV comprising a polynucleotide sequence not of AAV origin (e.g., a transgene). The term “AAV” includes AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), serotype rh10 AAV, serotype rh74 AAV, or a pseudotyped rAAV (e.g., AAV2/9, referring an AAV vector with the genome of AAV2 (e.g., the ITRs of AAV2) and the capsid of AAV9). In several embodiments, the preferred serotype for delivery to human patients is AAV6.

The term “AAV virion”, “AAV viral particle” or “rAAV vector particle” refers to a viral particle composed of at least AAV capsid protein and an encapsidated polynucleotide.

As used herein, the term “kit” may be used to describe variations of the portable, self-contained enclosure that includes at least one set of components to conduct one or more of the diagnostic or therapeutic methods of the present disclosure.

Terms and phrases used in this application, and variations thereof, including in the appended claims, unless otherwise expressly stated, should be construed as open ended as opposed to limiting. As examples of the foregoing, the term “including” should be read to mean ‘including, without limitation,’ ‘including but not limited to,’ or the like; the term “comprising” as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term “includes” should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but it may include additional steps. When used in the context of a compound, composition or device, the term “comprising” means that the compound, composition, or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% homologous or identical includes 96%, 97%, 98%, 99%, and 100% homologous or identical to the reference sequence. In addition, when a sequence is disclosed as “comprising” a nucleotide or amino acid sequence, such a reference shall also include, unless otherwise indicated, that the sequence “consists of” or “consists essentially of” the recited sequence. Likewise, when a composition is disclosed as “comprising” a feature, such a reference shall also include, unless otherwise indicated, that the composition “consists of” or “consists essentially of” the recited feature.

### Vectors and Expression Cassettes

Provided herein are rAAV vectors comprising expression cassettes containing a transgene coding sequence for a lymphocyte-recruiting chemokine. In some embodiments, the transgene is selected from CXCL9, MIP-1α/β, CXCL10, CXCL16, and variants thereof. In some embodiments, the expression cassette contains CXCL9 operably controlled by a neuron-specific promoter such as GFAP, and the rAAV vector is an rAAV2/6-GFAP-CXCL9 vector. The expression cassette is flanked by 3′ and 5′ inverted terminal repeats, such as AAV2 ITRs.

In some embodiments, the transgene encodes CXCL9. In some embodiments, the CXCL9 is a mouse CXCL9. In some embodiments, it is a human CXCL9.

In some embodiments, the transgene encodes MIP-1α/β, or CCL3. In some embodiments, the CXCL9 is a mouse MIP-1α/B (SEQ ID NO: 3). In some embodiments, it is a human MIP-1α/B (SEQ ID NO: 4). The transgene may comprises a nucleic acid sequence that is at least about 90%, 92.5%, 95%, 98%, or 99% identical to SEQ ID NO: 3 or 4. The transgene may comprises a nucleic acid sequence as set forth as SEQ ID NO: 3. The transgene may comprise the sequence of SEQ ID NO: 4.

```
MIP-1α/β (CCL3) Mus musculus sequence
(SEQ ID NO: 3)
    1 gggcatatgg cttcagacac cagaaggata caagcagcag cgagtaccag tcccttttct
  61 gttctgctga caagctcacc ctctgtcacc tgctcaacat catgaaggtc tccaccactg
121 cccttgctgt tcttctctgt accatgacac tctgcaacca agtcttctca gcgccatatg
181 gagctgacac cccgactgcc tgctgcttct cctacagccg gaagattcca cgccaattca
241 tcgttgacta ttttgaaacc agcagccttt gctcccagcc aggtgtcatt ttcctgacta
301 agagaaaccg gcagatctgc gctgactcca aagagacctg ggtccaagaa tacatcactg
361 acctggaact gaatgcctga gagtcttgga ggcagcgagg aaccccccaa acctccatgg
421 gtcccgtgta gagcaggggc ttgagccccg gaacattcct gccacctgca tagctccatc
481 tcctataagc tgtttgctgc caagtagcca catcgaggga ctcttcactt gaaattttat
541 ttaatttaat cctattggtt taatactatt taattttgta atttatttta ttgtcatact
601 tgtatttgtg actatttatt ctgaaagact tcaggacacg ttcctcaacc cccatctccc
661 tcccagttgg tcacactgtt tggtgacagc tattctaggt agacatgatg acaaagtcat
721 gaactgacaa atgtacaata gatgctttgt ttataccaga gaagtaataa atatgccctt
781 taacaagtga aaaaaaaaaa aaaa
MIP-1α/β (CCL3) Homo sapiens sequence
(SEQ ID NO: 4)
    1 ctctttaaga cttttatttt tatctctaga aggggtctta gccccctagt ctccagttgc
  61 tgctgacacg ccgaccgcct gctgcttcag ctacacctcc cggcagattc cacagaattt
121 catagctgac tactttgaga cgagcagcca gtgctccaag cccggtgtca tcttcctaac
181 caagcgaagc cggcaggtct gtgctgaccc cagtgaggag tgggtccaga aatatgtcag
241 cgacctggag ctgagtgcct gaggggtcca gaagcttcga ggcccagcga cctcggtggg
301 cccagtgggg aggagcagga gcctgagcct tgggaacatg cgtgtgacct ccacagctac
361 ctcttctatg gactggttgt tgccaaacag ccacactgtg ggactcttct taacttaaat
421 tttaatttat ttatactatt tagtttttgt aatttatttt cgatttcaca gtgtgtttgt
481 gattgtttgc tctgagagtt cccctgtccc ctcccccttc cctcacaccg cgtctggtga
541 caaccgagtg gctgtcatca gcctgtgtag gcagtcatgg caccaaagcc accagactga
601 caaatgtgta tcggatgctt ttgttcaggg ctgtgatcgg cctggggaaa taataaagat
661 gctcttttaa aaggtaaa
```

The transgene of the disclosed rAAV vectors may be flanked by two AAV inverted terminal repeat (ITR) sequences. The ITR sequences of the disclosed rAAV vectors may be derived from any AAV serotype (e.g., 1, 2, 3, 4, 5, 6, 7, 8, 9, or 10) or may be from more than one serotype. In some embodiments, the ITR sequences are from AAV2. In some embodiments, the ITR sequences are wild-type AAV2 ITR sequences. In particular embodiments, the rAAV vectors comprise CXCL9 flanked by wild-type AAV2 ITRs.

Accordingly, in some embodiments, the disclosure provides an rAAV vector comprising a CBA promoter operably linked to a CXCL9 transgene, wherein the AAV vector comprises AAV2 ITRs. In particular embodiments, the disclosure provides an rAAV vector comprising a CBA promoter operably linked to a CXCL9 transgene, wherein the AAV vector comprises AAV2 ITRs and is of a serotype AAV6. In some embodiments, the disclosure provides an rAAV vector comprising a GFAP promoter operably linked to a CXCL9 transgene, wherein the AAV vector comprises AAV2 ITRs. In particular embodiments, the disclosure provides an rAAV vector comprising a GFAP promoter operably linked to a CXCL9 transgene, wherein the AAV vector comprises AAV2 ITRs and is of a serotype AAV6.

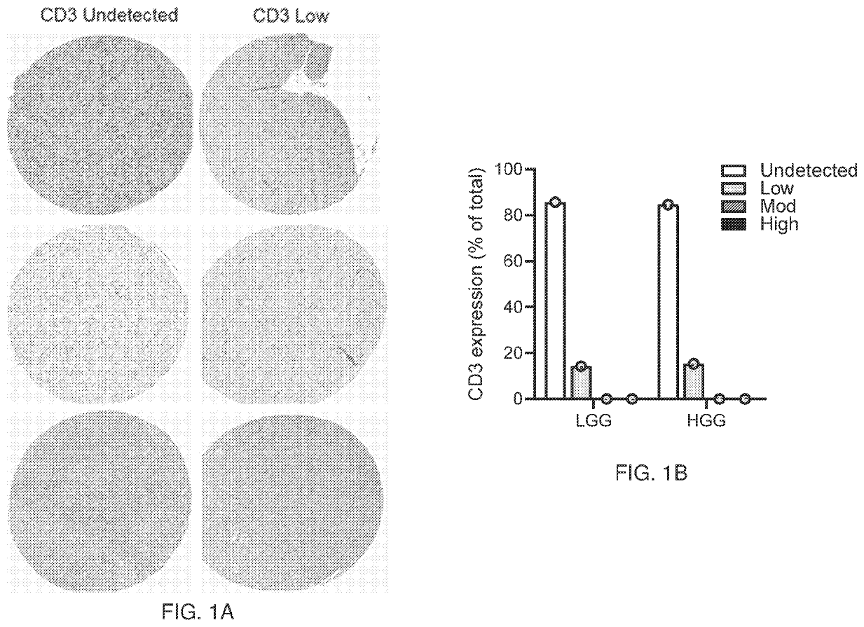

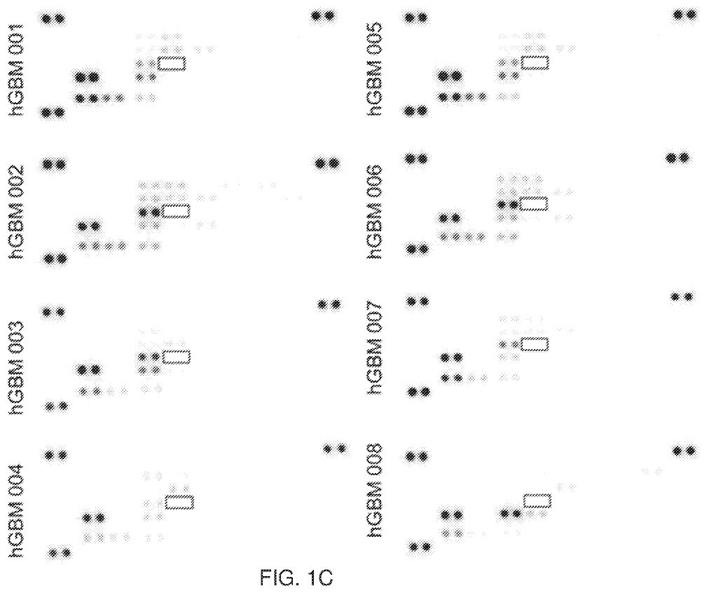

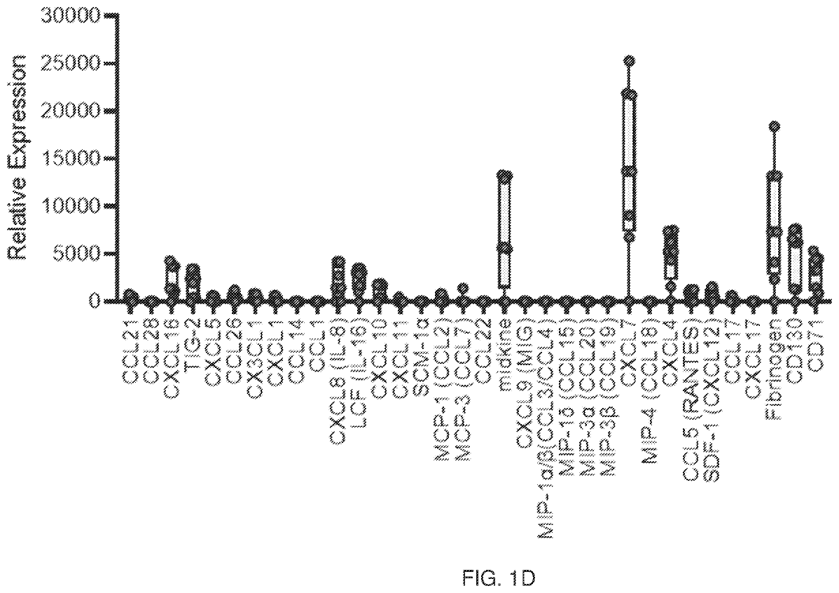

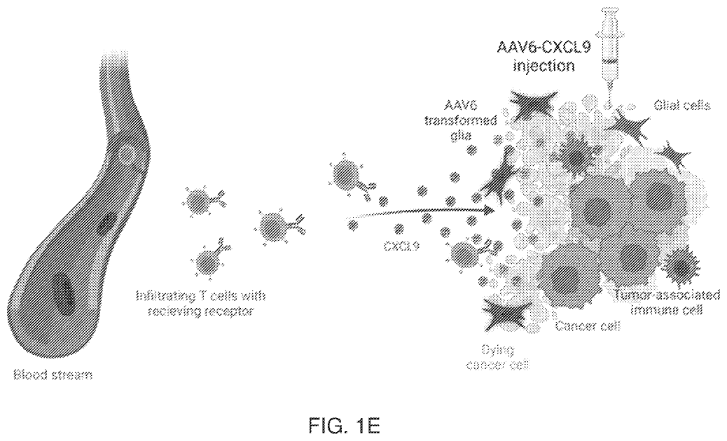

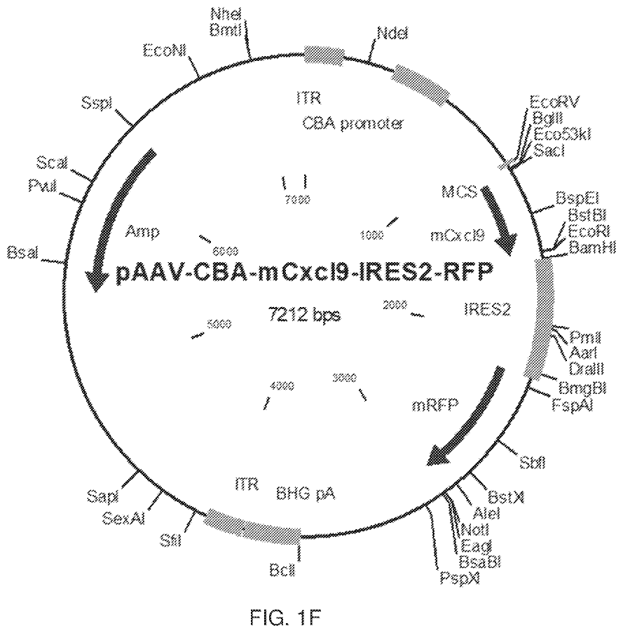

The expression cassette may comprise a nucleic acid sequence having at least 85%, 90%, 92.5%, 95%, 98%, or 99% identity to SEQ ID NO: 5, which corresponds to the pAAV-CBA-mCXCL9-IRES-RFP vector. The expression cassette may comprise the nucleic acid sequence of SEQ ID NO: 5. A map of this vector is provided in FIG. 1F.

(SEQ ID NO: 5)
TAGCTGCGCGCTCGCTCGCTCACTGAGGCCGCCCGGGCAAAGCCCGGGCGTCGGGCGACCTTTGGT
CGCCCGGCCTCAGTGAGCGAGCGAGCGCGCAGAGAGGGAGTGGCCAACTCCATCACTAGGGGTTC
CTTGTAGTTAATGATTAACCCGCCATGCTACTTATCTACGTAGCCATGCTCTAGGTACCATTGAC
GTCAATAATGACGTATGTTCCCATAGTAACGCCAATAGGGACTTTCCATTGACGTCAATGGGTGG
ACTATTTACGGTAAACTGCCCACTTGGCAGTACATCAAGTGTATCATATGCCAAGTACGCCCCCT
ATTGACGTCAATGACGGTAAATGGCCCGCCTGGCATTATGCCCAGTACATGACCTTATGGGACTT
TCCTACTTGGCAGTACATCTACGTATTAGTCATCGCTATTACCATGGTCGAGGTGAGCCCCACGT
TCTGCTTCACTCTCCCCATCTCCCCCCCCTCCCCACCCCCAATTTTGTATTTATTTATTTTTTAA
TTATTTTGTGCAGCGATGGGGGCGGGGGGGGGGGGGGGGCGCGCGCCAGGCGGGGCGGGGCGGGG
CGAGGGGGGGGGCGGGGCGAGGCGGAGAGGTGCGGCGGCAGCCAATCAGAGCGGCGCGCTCCGAA
AGTTTCCTTTTATGGCGAGGCGGCGGCGGCGGCGGCCCTATAAAAAGCGAAGCGCGCGGCGGGCG
GGAGTCGCTGCGACGCTGCCTTCGCCCCGTGCCCCGCTCCGCCGCCGCCTCGCGCCGCCCGCCCC
GGCTCTGACTGACCGCGTTACTCCCACAGGTGAGCGGGCGGGACGGCCCTTCTCCTCCGGGCTGT
AATTAGCGCTTGGTTTAATGACGGCTTGTTTCTTTTCTGTGGCTGCGTGAAAGCCTTGAGGGGCT
CCGGGAGGGCCCTTTGTGCGGGGGGAGCGGCTCGGGGCTGTCCGCGGGGGGACGGCTGCCTTCGG
GGGGGACGGGGCAGGGCGGGGTTCGGCTTCTGGCGTGTGACCGGCGGCTCTAGAGCCTCTGCTAA
CCATGTTCATGCCTTCTTCTTTTTCCTACAGCTCCTGGGCAACGTGCTGGTTATTGTGCTGTCTC
ATCATTTTGGCAAGAATTGGATCAAAGCTTCAATTGGATATCACTAGCGCTACCGGACTCAGATC
TCGAGCTCAATGAAGTCCGCTGTTCTTTTCCTTTTGGGCATCATCTTCCTGGAGCAGTGTGGAGT
TCGAGGAACCCTAGTGATAAGGAATGCACGATGCTCCTGCATCAGCACCAGCCGAGGCACGGTCC
ACTACAAATCCCTCAAAGACCTCAAACAGTTTGCCCCAAGCCCCAATTGCAACAAAACTGAAATC
ATTGCTACACTGAAGAACGGAGATCAAACCTGCCTAGATCCGGACTCGGCAAATGTGAAGAAGCT
GATGAAAGAATGGGAAAAGAAGATCAACCAAAAGAAAAAGCAAAAGAGGGGGAAAAAACATCAAA
AGAACATGAAAAACAGAAAACCCAAAACACCCCAAAGTCGTCGTCGTTCAAGGAAGACTACAGAT
TACAAGGATGACGACGATAAGTAAAGCTTCGAATTCTGCAGTCGACGGTACCGCGGGCCCGGGAT
CCGCCCCTCTCCCTCCCCCCCCCCTAACGTTACTGGCCGAAGCCGCTTGGAATAAGGCCGGTGTG
CGTTTGTCTATATGTTATTTTCCACCATATTGCCGTCTTTTGGCAATGTGAGGGCCCGGAAACCT
GGCCCTGTCTTCTTGACGAGCATTCCTAGGGGTCTTTCCCCTCTCGCCAAAGGAATGCAAGGTCT
GTTGAATGTCGTGAAGGAAGCAGTTCCTCTGGAAGCTTCTTGAAGACAAACAACGTCTGTAGCGA
CCCTTTGCAGGCAGCGGAACCCCCCACCTGGCGACAGGTGCCTCTGCGGCCAAAAGCCACGTGTA
TAAGATACACCTGCAAAGGCGGCACAACCCCAGTGCCACGTTGTGAGTTGGATAGTTGTGGAAAG
AGTCAAATGGCTCTCCTCAAGCGTATTCAACAAGGGGCTGAAGGATGCCCAGAAGGTACCCCATT
GTATGGGATCTGATCTGGGGCCTCGGTGCACATGCTTTACATGTGTTTAGTCGAGGTTAAAAAAA
CGTCTAGGCCCCCCGAACCACGGGGACGTGGTTTTCCTTTGAAAAACACGATGATAATATGGCCT
CCTCCGAGGACGTCATCAAGGAGTTCATGCGCTTCAAGGTGCGCATGGAGGGCTCCGTGAACGGC
CACGAGTTCGAGATCGAGGGCGAGGGCGAGGGCCGCCCCTACGAGGGCACCCAGACCGCCAAGCT
GAAGGTGACCAAGGGCGGCCCCCTGCCCTTCGCCTGGGACATCCTGTCCCCTCAGTTCCAGTACG
GCTCCAAGGCCTACGTGAAGCACCCCGCCGACATCCCCGACTACTTGAAGCTGTCCTTCCCCGAG
GGCTTCAAGTGGGAGCGCGTGATGAACTTCGAGGACGGCGGCGTGGTGACCGTGACCCAGGACTC
CTCCCTGCAGGACGGCGAGTTCATCTACAAGGTGAAGCTGCGCGGCACCAACTTCCCCTCCGACG
GCCCCGTAATGCAGAAGAAGACCATGGGCTGGGAGGCCTCCACCGAGCGGATGTACCCCGAGGAC
GGCGCCCTGAAGGGCGAGATCAAGATGAGGCTGAAGCTGAAGGACGGCGGCCACTACGACGCCGA
GGTCAAGACCACCTACATGGCCAAGAAGCCCGTGCAGCTGCCCGGCGCCTACAAGACCGACATCA
AGCTGGACATCACCTCCCACAACGAGGACTACACCATCGTGGAACAGTACGAGCGCGCCGAGGGC
CGCCACTCCACCGGCGCCCTGTACAAGTAAAGCGGCCGCGACTCTAGATCATAATCAGCCATACC
ACATTTGTAGAGGTTTTACTTGCTTTAAAAAACCTCCCACACCTCCCCCTGAACCTGAAACATAA
AATGAATGCAATTCCTCGAGCAGCTTATCGATAATCAACCTCTGGATTACAAAATTTGTGAAAGA
TTGACTGGTATTCTTAACTATGTTGCTCCTTTTACGCTATGTGGATACGCTGCTTTAATGCCTTT
GTATCATGCTATTGCTTCCCGTATGGCTTTCATTTTCTCCTCCTTGTATAAATCCTGGTTGCTGT
CTCTTTATGAGGAGTTGTGGCCCGTTGTCAGGCAACGTGGCGTGGTGTGCACTGTGTTTGCTGAC
GCAACCCCCACTGGTTGGGGCATTGCCACCACCTGTCAGCTCCTTTCCGGGACTTTCGCTTTCCC
CCTCCCTATTGCCACGGCGGAACTCATCGCCGCCTGCCTTGCCCGCTGCTGGACAGGGGCTCGGC
TGTTGGGCACTGACAATTCCGTGGTGTTGTCGGGGAAATCATCGTCCTTTCCTTGGCTGCTCGCC
TGTGTTGCCACCTGGATTCTGCGCGGGACGTCCTTCTGCTACGTCCCTTCGGCCCTCAATCCAGC
GGACCTTCCTTCCCGCGGCCTGCTGCCGGCTCTGCGGCCTCTTCCGCGTCTTCGCCTTCGCCCTC
AGACGAGTCGGATCTCCCTTTGGGCCGCCTCCCCGCATCGATACCGTCGACTCGCTGATCAGCCT
CGACTGTGCCTTCTAGTTGCCAGCCATCTGTTGTTTGCCCCTCCCCCGTGCCTTCCTTGACCCTG
GAAGGTGCCACTCCCACTGTCCTTTCCTAATAAAATGAGGAAATTGCATCGCATTGTCTGAGTAG
GTGTCATTCTATTCTGGGGGGTGGGGTGGGGCAGGACAGCAAGGGGGAGGATTGGGAAGACAATA
GCAGGCATGCTGGGGATGCGGTGGGCTCTATGGCTTCTGAGGCGGAAAGAACCAGCTGGGGCTCG
ACTAGAGCATGGCTACGTAGATAAGTAGCATGGCGGGTTAATCATTAACTACAAGGAACCCCTAG
TGATGGAGTTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGCTTTTTGCAA
AAGCCTAGGCCTCCAAAAAAGCCTCCTCACTACTTCTGGAATAGCTCAGAGGCCGAGGCGGCCTC
GGCCTCTGCATAAATAAAAAAAATTAGTCAGCCATGGGGCGGAGAATGGGCGGAACTGGGCGGAG
TTAGGGGCGGGATGGGCGGAGTTAGGGGGGGGACTATGGTTGCTGACTAATTGAGATGCATGCTT
TGCATACTTCTGCCTGCTGGGGAGCCTGGGGACTTTCCACACCTGGTTGCTGACTAATTGAGATG
CATGCTTTGCATACTTCTGCCTGCTGGGGAGCCTGGGGACTTTCCACACCCTAACTGACACACAT
TCCACAGCTGCATTAATGAATCGGCCAACGCGCGGGGAGAGGCGGTTTGCGTATTGGGCGCTCTT
CCGCTTCCTCGCTCACTGACTCGCTGCGCTCGGTCGTTCGGCTGCGGCGAGCGGTATCAGCTCAC
TCAAAGGCGGTAATACGGTTATCCACAGAATCAGGGGATAACGCAGGAAAGAACATGTGAGCAAA
AGGCCAGCAAAAGGCCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACCCGACAGGACTATAA
AGATACCAGGCGTTTCCCCCTGGAAGCTCCCTCGTGCGCTCTCCTGTTCCGACCCTGCCGCTTAC
CGGATACCTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGTTCAGCCC
GACCGCTGCGCCTTATCCGGTAACTATCGTCTTGAGTCCAACCCGGTAAGACACGACTTATCGCC
ACTGGCAGCAGCCACTGGTAACAGGATTAGCAGAGCGAGGTATGTAGGCGGTGCTACAGAGTTCT
TGAAGTGGTGGCCTAACTACGGCTACACTAGAAGAACAGTATTTGGTATCTGCGCTCTGCTGAAG
CCAGTTACCTTCGGAAAAAGAGTTGGTAGCTCTTGATCCGGCAAACAAACCACCGCTGGTAGCGG
TGGTTTTTTTGTTTGCAAGCAGCAGATTACGCGCAGAAAAAAAGGATCTCAAGAAGATCCTTTGA
TCTTTTCTACGGGGTCTGACGCTCAGTGGAACGAAAACTCACGTTAAGGGATTTTGGTCATGAGA
TTATCAAAAAGGATCTTCACCTAGATCCTTTTAAATTAAAAATGAAGTTTTAAATCAATCTAAAG
TATATATGAGTAAACTTGGTCTGACAGTTACCAATGCTTAATCAGTGAGGCACCTATCTCAGCGA
TCTGTCTATTTCGTTCATCCATAGTTGCCTGACTCCCCGTCGTGTAGATAACTACGATACGGGAG
GGCTTACCATCTGGCCCCAGTGCTGCAATGATACCGCGAGACCCACGCTCACCGGCTCCAGATTT
ATCAGCAATAAACCAGCCAGCCGGAAGGGCCGAGCGCAGAAGTGGTCCTGCAACTTTATCCGCCT
CCATCCAGTCTATTAATTGTTGCCGGGAAGCTAGAGTAAGTAGTTCGCCAGTTAATAGTTTGCGC
AACGTTGTTGCCATTGCTACAGGCATCGTGGTGTCACGCTCGTCGTTTGGTATGGCTTCATTCAG
CTCCGGTTCCCAACGATCAAGGCGAGTTACATGATCCCCCATGTTGTGCAAAAAAGCGGTTAGCT
CCTTCGGTCCTCCGATCGTTGTCAGAAGTAAGTTGGCCGCAGTGTTATCACTCATGGTTATGGCA
GCACTGCATAATTCTCTTACTGTCATGCCATCCGTAAGATGCTTTTCTGTGACTGGTGAGTACTC
AACCAAGTCATTCTGAGAATAGTGTATGCGGCGACCGAGTTGCTCTTGCCCGGCGTCAATACGGG
ATAATACCGCGCCACATAGCAGAACTTTAAAAGTGCTCATCATTGGAAAACGTTCTTCGGGGCGA
AAACTCTCAAGGATCTTACCGCTGTTGAGATCCAGTTCGATGTAACCCACTCGTGCACCCAACTG
ATCTTCAGCATCTTTTACTTTCACCAGCGTTTCTGGGTGAGCAAAAACAGGAAGGCAAAATGCCG
CAAAAAAGGGAATAAGGGCGACACGGAAATGTTGAATACTCATACTCTTCCTTTTTCAATATTAT
TGAAGCATTTATCAGGGTTATTGTCTCATGAGCGGATACATATTTGAATGTATTTAGAAAAATAA
ACAAATAGGGGTTCCGCGCACATTTCCCCGAAAAGTGCCACCTGACGTCTAAGAAACCATTATTA
TCATGACATTAACCTATAAAAATAGGCGTATCACGAGGCCCTTTCGTCTCGCGCGTTTCGGTGAT
GACGGTGAAAACCTCTGACACATGCAGCTCCCGGAGACGGTCACAGCTTGTCTGTAAGCGGATGC
CGGGAGCAGACAAGCCCGTCAGGGCGCGTCAGCGGGTGTTGGCGGGTGTCGGGGCTGGCTTAACT
ATGCGGCATCAGAGCAGATTGTACTGAGAGTGCACCATTCGACGCTCTCCCTTATGCGACTCCTG
CATTAGGAAGCAGCCCAGTAGTAGGTTGAGGCCGTTGAGCACCGCCGCCGCAAGGAATGGTGCAT
GCAAGGAGATGGCGCCCAACAGTCCCCCGGCCACGGGGCCTGCCACCATACCCACGCCGAAACAA
GCGCTCATGAGCCCGAAGTGGCGAGCCCGATCTTCCCCATCGGTGATGTCGGCGATATAGGCGCC
AGCAACCGCACCTGTGGCGCCGGTGATGCCGGCCACGATGCGTCCGGCGTAGAGGATCTGGCTAG
CGATGACCCTGCTGATTGGTTCGCTGACCATTTCCGGGTGCGGGACGGCGTTACCAGAAACTCAG
AAGGTTCGTCCAACCAAACCGACTCTGACGGCAGTTTACGAGAGAGATGATAGGGTCTGCTTCAG
TAAGCCAGATGCTACACAATTAGGCTTGTACATATTGTCGTTAGAACGCGGCTACAATTAATACA
TAACCTTATGTATCATACACATACGATTTAGGTGACACTATAGAATACACGGAATTAATTC

### Treatment of Solid Tumors

In various aspects, methods of treatment of solid tumors are provided. Solid tumors of the CNS such as GBM and gliomas may be particularly suitable for treatment by any of the disclosed methods. In some embodiments, a non-brain solid tumor that has metastasized to the brain is suitable for treatment by any of the disclosed methods. Injection of the disclosed rAAV particles directly into the tumor are contemplated.

Provided herein are methods of transducing cells of the tumor or that interact with the tumor microenvironment. In some embodiments, methods of contacting a neuron, glial cell or astrocyte with any of the disclosed rAAV particles or compositions are provided. In some embodiments, the contacting is in vivo. In some embodiments, the contacting is in vitro or ex vivo.

Glioblastoma multiforme (GBM), the most common and fatal form of a primary brain tumor, accounts for approximately 60% of all glioma cases and is categorized as grade IV glioma. Local invasiveness, neoangiogenesis, and intratumor heterogeneity are among the most important hallmarks of the aggressiveness of GBM. GBM tumors contain functional subsets of cells called glioblastoma stem-like cells (GSCs), which are resistant to radiation therapy and chemotherapy and eventually lead to tumor recurrence. Recent studies showed that GSCs reside in particular tumor niches (such as perivascular niches) that are necessary to support their behavior. The presence of GSCs was first demonstrated by the identification of a CD133+ cell subpopulation that is capable of tumor initiation in vivo (Singh et al., 2004). CD133, a glycoprotein cell surface marker of normal neural stem cells, is commonly used to distinguish GSCs. The percentage of CD133+ cells may be significantly higher in recurrent GBMs after radiotherapy and chemotherapy as compared with primary tumors. Apart from CD133, other cell surface markers, such as SSEA-1 and CD44, have been used to enrich or distinguish stem-like populations in GBM. In particular, CD44 is highly expressed in the mesenchymal subtype of GBM, and its expression has been used to enrich for stem-like cells (Anido et al., 2010). It has been reported that Cd44−/− and Cd44+/− mice survived longer than Cd44+/+ mice with PDGF-driven gliomas, indicating that CD44 actively contributes to aggressive glioma growth (Pietras et al., Cell Stem Cell. 2014). It was recently shown that patient-derived glioma stem-like cells exhibited a characteristic equilibrium of distinct CD133+ and CD44+ subpopulations (Brown et al., PLOS One. 2017; 12 (2): e0172791, herein incorporated by reference). GSC populations are further characterized by their rate of growth and mvoement, i.e., fast-cycling (fast-dividing and fast-moving, or simply “fast”) and slow-cycling (slow-dividing and slow-moving, or simply “slow”) populations (see id.).

Combination therapies that include any of the disclosed rAAV particle compositions and compositions comprising an immune modulator are described. Exemplary immune modulators include immune checkpoint inhibitors. Further contemplated are combination therapies with CAR-T engineered cell immunotherapies, and other adoptive cell imunotherapies. CAR-T cells are known to modify tumor microenvironments through the reprogramming ex vivo of the subject's T cells to recognize and attack a tumor-specific antigen (such as CD19). Other immune modulators are contemplated for use in the disclosed therapeutic methods and kits, such as monoclonal antibody treatments, RNA nanoparticle vaccines, dendritic cell vaccines, and small-molecule checkpoint inhibitors.

In some embodiments, the immune checkpoint inhibitor is an antagonist of PD-1, PD-L1, CTLA-4, TIM-3, VISTA, LAG-3, B7-H3, or another immune checkpoint protein. In some embodiments, the immune checkpoint inhibitor is a PD-1 antagonist. In some embodiments, the immune checkpoint inhibitor is an anti-PD-1 antibody, such as nivolumab, pembrolizumab, avelumab, and durvalumab. In some embodiments, the immune checkpoint inhibitor is an anti-PD-1 antibody that is not (yet) FDA-approved.

The disclosed rAAV compositions are well-suited for use in combination therapies because they modify the TME to make the tumor more vulnerable to immune modulators, and thus enhance their efficacy. For example, administration of any of the disclosed rAAV particles results in sensitization of CNS tumors to treatment by a PD-1 antagonist (or a CTLA-4 antagonist). In some embodiments, a PD-1 antagonist is administered in a dose of 10 mg/kg of the subject. In some embodiments, the antagonist is administered intravenously. In some embodiments, the antagonist is administered intrathecally or orally.

The composition comprising an immune modulator be administered concurrently with the rAAV particle composition. In other embodiments, it is administered before the rAAV composition, or after the rAAV composition. In some embodiments, an immune checkpoint inhibitor is administered within 1 day, 3 days, 5 days, 1 week, 2 weeks, or 3 weeks of the rAAV composition administration. Contemplated herein is the use of the rAAV particle as a neo-adjuvant, i.e., a treatment given first to sensitize the tumor (or TME) to treatment by the primary therapy—the immune checkpoint inhibitor. In this manner, the disclosed rAAV therapies are used to improve efficacies of immune checkpoint therapies such as PD-1 monoclonal antibody therapies.

Following administration of the combination therapy to a subject in need thereof, the subject may be monitored for the presence of biomarkers indicating a positive response to the rAAV-chemokine treatment. Exemplary biomarkers that may indicate this response include adiponectin, ClqR1, CCL5, CCL7, CCL12 (MCP-1), CD40, CXCL10, CXCL16, lipocalin-2, IL-1β, TNFα, myeloperoxidase, IFNα, and IFNβ.

In exemplary embodiments, methods of treating a subject suffering from glioma or GBM, the method comprising administering a therapeutically effective amount of an rAAV particle comprising (i) a nucleic acid vector comprising an expression cassette comprising a transgene encoding a chemokine, wherein the chemokine is CXCL9 or a variant thereof, wherein the transgene is operably linked to a promoter, and wherein the expression cassette is flanked on each side by an ITR of serotype 2. In some embodiments, the CXCL9 chemokine is a human CXCL9. In some embodiments, the capsid protein is of serotype 6. In some embodiments, this administration results in expression of a therapeutically effective amount of human CXCL9, thereby treating the glioma or GBM. In exemplary embodiments, the methods further comprise administering an immune modulator, such as an anti-PD1 antagonist.

### Regulatory Elements

These vectors may comprise a heterologous promoter driving expression of the polynucleotide, such as a promoter selected from a CD68 promoter, a Glial Fibrillary Acidic protein (GFAP) promoter, a CMV enhancer/chicken Beta-actin (CAG) promoter, a Microtubule-associated protein 2 (MAP2) promoter or a synapsin (SYN) promoter. In various embodiments, the heterologous promoter is active in astrocyte cells and/or microglial cells.

Aspects of this disclosure relate to the delivery of CPRs and/or modified cell expressing a CPR to glial cells of the CNS. Likewise, aspects of this disclosure relate to the engineering of modified glial cells for delivery to a subject suffering from a neurodegenerative disease. In particular embodiments, the disclosure provides methods of preparing modified astrocytes that express any of the disclosed CPRs. Astrocytes are macroglial cells in the CNS that help provide physical structure and metabolic support to neurons in the brain. In some embodiments, the disclosure provides improved methods of delivery of these CPRs to astrocytes, including the delivery of expression vectors encoding CPRs to glial cells such as astrocytes using a recombinant AAV technique.

To achieve appropriate or enhanced expression levels of the CPR, any of a number of heterologous promoters suitable for use in the selected host cell may be employed. The promoter may be, for example, a constitutive promoter, tissue-specific promoter, inducible promoter, or a synthetic promoter. Inducible promoters and/or regulatory elements may also be contemplated for achieving appropriate expression levels of the heterologous peptide. Non-limiting examples of suitable inducible promoters include the CBA promoter and those promoters from genes such as cytochrome P450 genes, heat shock protein genes, metallothionein genes, and hormone-inducible genes, such as the estrogen gene promoter.

Tissue-specific promoters and/or regulatory elements are also contemplated herein. In certain embodiments, the heterologous promoters of the disclosed chimeric receptors are active in brain cells, such as human brain cells (e.g. human neurons and glia). In various embodiments, the heterologous promoter is active in astrocyte cells. In some embodiments, the heterologous promoter is active in microglial cells. The disclosed vectors may comprise a heterologous promoter selected from a CD68 promoter, a Glial Fibrillary Acidic protein (GFAP) promoter, a CD8 promoter, a CMV enhancer/chicken Beta-actin (CAG) promoter, a Microtubule-associated protein 2 (MAP2) promoter or a synapsin 1 (SYN) promoter. In particular embodiments, the promoter is a CD68 promoter. In some embodiments, the promoter is a GFAP promoter. In some embodiments, the promoter is a CD8 promoter.

Additional exemplary promoters active in human brain cells include, but are not limited to, the human synapsin 1 gene promoter (SYN), the hybrid CMV enhancer/chicken β-actin (CAG) promoter, Glial Fibrillary Acidic protein (GFAP) promoter, Microtubule-associated protein 2 (MAP2) promoter, and the platelet-derived growth factor-β chain promoter (1500 bp). These promoters are described in, e.g., Kugler et al. Virology, 311 (1): 89-95 (2003) and Morelli et al. J Gen Virol., 80 (Pt 3): 571-83 (1999), which are incorporated herein by reference. Non-limiting examples of such promoters that may be used include species-specific promoters, such as human-specific promoters.

Additional examples of neuron-specific promoters are known in the art. Non-limiting examples of neuron-specific promoters include neuron-specific enolase promoter, an aromatic amino acid decarboxylase promoter, a neurofilament promoter, a synapsin promoter, a thy-1 promoter, a serotonin receptor promoter, a GnRH promoter, an L7 promoter, a DNMT promoter, an enkephalin promoter, a myelin basic protein promoter, a CMV enhancer/platelet-derived growth factor-β promoter, a motor neuron-specific gene Hb9 promoter, and an alpha subunit of Ca (2+)-calmodulin-dependent protein kinase II promoter. In some embodiments, the promoter is mouse MECP2. In some embodiments, the promoter is JeT. In some embodiments, the promoter is human synapsin. In some embodiments, the promoter is a minimal core promoter of the thymidine kinase gene (minTK) (see, e.g., Jiang et al. (2008). Regulation of the MAD1 promoter by G-CSF. Nuc Acids Res. 36, (5): 1517-1531 which is incorporated by reference in its entirety herein). In some embodiments, the promoter is a minimal cytomegalovirus (minCMV) promoter (see, e.g., Ede et al. (2016). Quantitative Analyses of Core Promoters Enable Precise Engineering of Regulated Gene Expression in Mammalian Cells. ACS Synth Biol. 5 (5): 395-404 which is incorporated by reference in its entirety herein). In treating cancers as provided for herein, promoters are advantageous at least due to the reduced possibility of off-target expression of the transgene, thereby effectively increasing the delivered dose to the nervous system tissues and enhancing therapy. Non-limiting examples of expression regulatory sequences include promoters, insulators, silencers, response elements, introns (e.g., minimal minute virus (MVMi) introns), enhancers, initiation sites, termination signals, and polyA tails. Any combination of such regulatory sequences is contemplated herein (e.g., a promoter and an enhancer).

In some embodiments, the promoter is mouse MECP2. In some embodiments, the promoter is JeT. In some embodiments, the promoter is human synapsin. In some embodiments, the promoter is a modified human synapsin. In some embodiments, the promoter is IBA1.

Synthetic promoters are also contemplated herein. A synthetic promoter may comprise, for example, regions of known promoters, regulatory elements, transcription factor binding sites, enhancer elements, repressor elements, and the like.

Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

In some embodiments, one or more promoters may be operably linked to a coding nucleotide sequence in the heterologous nucleic acid. A promoter is “operably linked” to a nucleotide sequence when the promoter sequence controls and/or regulates the transcription of the nucleotide sequence. A promoter may be a constitutive promoter, tissue-specific promoter, an inducible promoter, or a synthetic promoter.

For example, constitutive promoters of different strengths can be used. A nucleic acid vector described herein may include one or more constitutive promoters, such as viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A cytomegalovirus (CMV) promoters. Non-limiting examples of constitutive mammalian promoters include various housekeeping gene promoters, as exemplified by the β-actin promoter (e.g., chicken β-actin promoter) and human elongation factor-1α (EF-1α) promoter. In some embodiments, chimeric viral/mammalian promoters may include a chimeric CMV/chicken beta actin (CBA, CB, or CAG) promoter. In some embodiments, a shortened or truncated promoter is used.

Inducible promoters are also be contemplated for achieving appropriate expression levels of the transgene. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

It is to be understood that a promoter may be a fragment of any one of the promoters disclosed herein, or one that retains partial promoter activity (e.g., 10-90, 30-60, 50-80, 80-99 or 90-99.9% of the activity) of a whole promoter.

In some embodiments, the rAAV vectors of the present disclosure further comprise an Internal Ribosome Entry Site (IRES). An IRES is a nucleotide sequence that allows for translation initiation in the middle of a messenger RNA (mRNA) sequence as part of the greater process of protein synthesis. Usually, in eukaryotes, translation can be initiated only at the 5′ end of the mRNA molecule, since 5′ cap recognition is required for the assembly of the initiation complex. In some embodiments, the IRES is located between the transgenes.

Other regulatory elements may also be contemplated for achieving appropriate expression levels of the heterologous nucleic acid. For examples, any of the disclosed rAAV vector may comprise a Woodchuck Hepatitis VirusPosttranscriptional Regulatory element (WPRE). A WPRE is a DNA sequence that, when transcribed, forms a tertiary structure that enhances expression of the vector. In some embodiments, the construct comprises a polyA sequence (or tail), such as a minimal polyA. Exemplary minimal polyA signals include bovine growth hormone (bGH) and SV40 polyA tails.

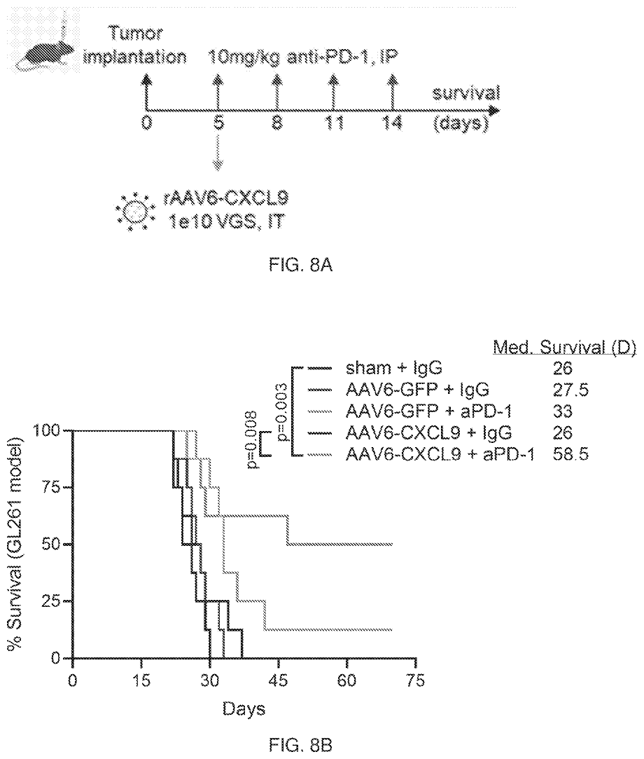

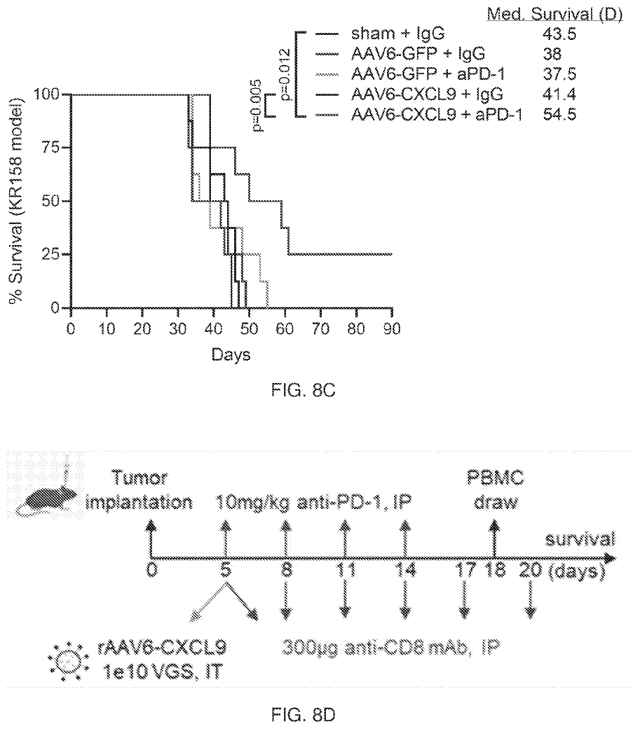

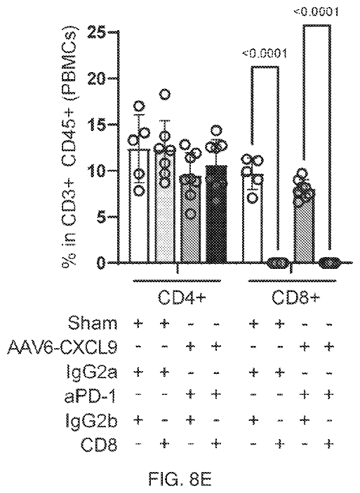

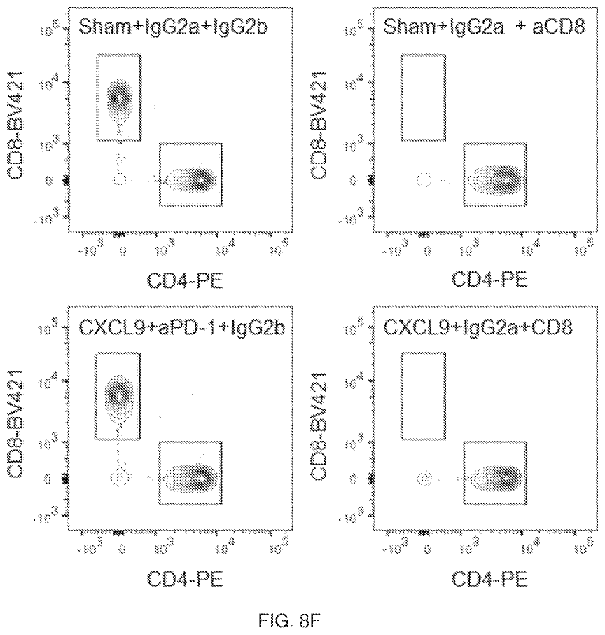

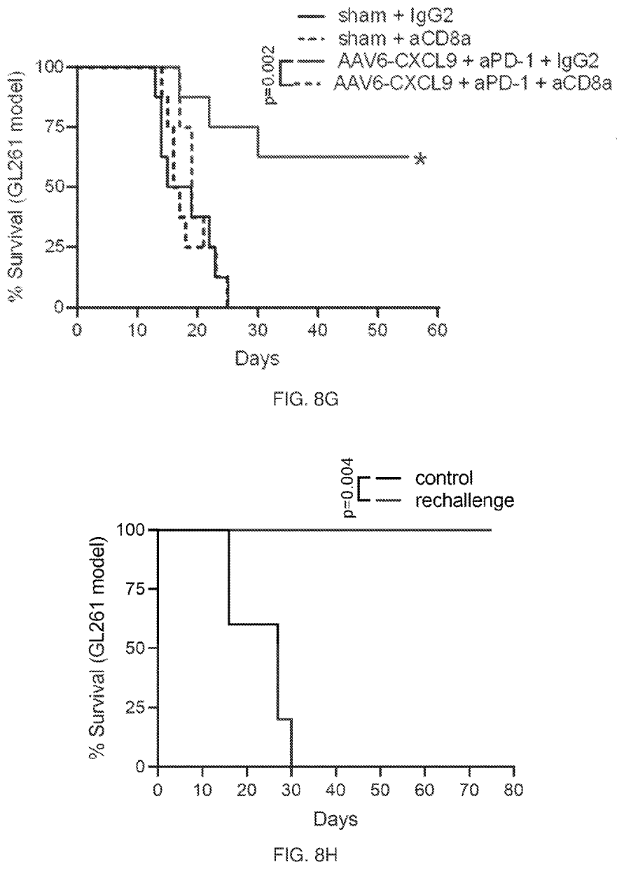

Accordingly, provided herein are rAAV particles comprising any of the disclosed vectors, for instance a vector containing an GFAP promoter, and further comprising a PHP.eB capsid (see FIG. 8). Further provided herein are rAAV particles comprising any of the disclosed vectors, for instance a vector containing a CD8 promoter, and further comprising a TM6 capsid. These rAAV particles may be designed for delivery to astrocytes, monocytes, or microglia, e.g., in modified cell or in a pharmaceutical composition or nanoparticle. In some embodiments, rAAV particles containing a GFAP promoter are delivered to astrocytes. Exemplary such particles may be referred to as “rAAV-PHP.eB-GFAP-CPR.” In some embodiments, rAAV particles containing a CD8 promoter are delivered to microglia and/or monocytes. Exemplary such particles may be referred to as “rAAV-TM6-CD8-CPR,” or simply “rAAV-TM6.”

Further provided herein are rAAV particles comprising any of the disclosed vectors, for instance a vector containing a CD8 promoter, and further comprising an AAV triple mutant (TM2) capsid. These rAAV particles may be designed for delivery to astrocytes, monocytes, or microglia, e.g., in modified cell or in a pharmaceutical composition or nanoparticle. In some embodiments, rAAV particles containing a GFAP promoter are delivered to astrocytes. Exemplary such particles may be referred to as “rAAV2/6-GFAP-CXCL9”, “rAAV2/6-CBA-CXCL9”, “rAAV-GFAP-CXCL9,” or simply “rAAV-CXCL9.”

### Pharmaceutical Compositions

In some aspects, provided herein are pharmaceutical compositions that comprise a viral vector as disclosed herein, and further comprise a pharmaceutical excipient, and may be formulated for administration to host cell ex vivo or in situ in an animal, and particularly a human. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions, diseases or disorders as described herein.

Formulations comprising pharmaceutically-acceptable excipients and/or carrier solutions are well-known to those of skill in the art, as is the development of suitable dosing and treatment regimens for using the particular compositions described herein in a variety of treatment regimens, including e.g., intracranial and ICV administration and formulation.

Typically, these formulations may contain at least about 0.1% of the therapeutic agent (e.g., therapeutic rAAV particle or preparation) or more, although the percentage of the active ingredient(s) may, of course, be varied and may conveniently be between about 1 or 2% and about 70% or 90% or more of the weight or volume of the total formulation. Naturally, the amount of therapeutic agent(s) in each therapeutically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art when preparing such pharmaceutical formulations. Additionally, a variety of dosages and treatment regimens may be desirable.

The term “excipient” refers to a diluent, adjuvant, carrier, or vehicle with which the rAAV particle or preparation, or composition of modified cells is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

In certain embodiments, the present disclosure provides a method of treating cancer comprisingadministering to the subject a composition comprising any of the disclosed rAAV particles and a pharmaceutically acceptable excipient. In some embodiments, the subject has been previously administered a composition comprising rAAV particles. In some embodiments, the subject has not been previously administered a composition comprising rAAV particles. In particular embodiments, the subject is a human.

In some embodiments, the number of rAAV particles administered to a subject may be on the order ranging from 106 to 1014 particles/mL or 103 to 1013 particles/mL, or any values therebetween for either range, such as for example, about 106, 107, 108, 109, 1010, 1011, 1012, 1013, or 1014 particles/mL. In one embodiment, rAAV particles of higher than 1013 particles/mL are be administered. In some embodiments, the number of rAAV particles administered to a subject may be on the order ranging from 106 to 10¹⁴ vgs/mL or 103 to 10¹⁵ vgs/mL, or any values there between for either range, such as for example, about 106, 107, 108, 109, 1010, 1011, 1012, 1013, or 10¹⁴ vgs/mL. In certain embodiments, the disclosed methods comprise administration of rAAV particle compositions in doses of 1×1010-4×1010 vgs/mL.

In certain embodiments, the disclosed methods comprise administration of rAAV particle compositions in doses of 1×109-1×1010 vgs per animal (or human) subject. In certain embodiments, the disclosed methods comprise administration of rAAV particle compositions in doses of 1×109-1×1011 vgs per animal subject. In some embodiments, rAAV particles of higher than 1011 vgs/mL are administered. In some embodiments, rAAV particles in amounts of lower than 109 vgs/mL are administered.

The rAAV particles can be administered as a single dose, or divided into two or more administrations as may be required to achieve therapy of the particular disease or disorder being treated. In some embodiments, 0.0001 mL to 10 mL are delivered to a subject.

In some embodiments, the number of rAAV particles administered to a host cell may be on the order ranging from 500 to 5,000 vector genomes (vgs)/cell. In particular embodiments, the disclosed methods comprise administration of rAAV particles in doses of about 500 vgs/cell, 1000 vgs/cell, 2000 vgs/cell, 3000 vgs/cell, 4000 vgs/cell, 5000 vgs/cell, 6000 vgs/cell or 7000 vgs/cell.

In some embodiments, the disclosure provides formulations of compositions disclosed herein in pharmaceutically acceptable solutions for administration to a cell or an animal, either alone or in combination with one or more other modalities of therapy, and in particular, for therapy of human cells, tissues, and diseases affecting man.

If desired, rAAV particle or preparation and compositions may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles or preparations and compositions of modified cells may thus be delivered along with various other agents as required in the particular instance. Such compositions may be purified from host cells or other biological sources, or alternatively may be chemically synthesized as described herein.

Typically, these formulations may contain at least about 0.1% of the therapeutic agent (e.g., rAAV particle or preparation and/or composition of modified cells) or more, although the percentage of the active ingredient(s) may, of course, be varied and may conveniently be between about 1 or 2% and about 70% or 80% or more of the weight or volume of the total formulation. Naturally, the amount of therapeutic agent(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it is desirable to deliver the rAAV particles or preparations and/or compositions of modified cells in suitably formulated pharmaceutical compositions disclosed herein either intracranially, intravenously, intracerebro-ventricularly, parenterally, intraocularly, intramuscularly, intrathecally, orally, intraperitoneally, intraparenchymally, (intra) cisternaly, intrathalamically, intrathoracically, by oral or nasal inhalation, or by direct injection to one or more cells, tissues, or organs. In particular embodiments, the disclosed rAAV particle compositions are formulated for intracranial, ICV, intrathecal, or IV delivery.

The pharmaceutical forms of the compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The pharmaceutical compositions of the present disclosure can be administered to the subject being treated by standard routes including, but not limited to, pulmonary, intranasal, oral, inhalation, parenteral such as intravenous, intraocular, ICV, topical, transdermal, intradermal, transmucosal, intraperitoneal, intramuscular, intracapsular, intraorbital, intravitreal, intracardiac, transtracheal, subcutaneous, subcuticular, intraarticular, subcapsular, subarachnoid, intraspinal, epidural and intrasternal injection.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

In some embodiments, a cell, tissue or organ is transduced in vivo, for example, for the purposes of treating a disease. In some embodiments, a cell, tissue or organ of the CNS is transduced in vivo. In some embodiments, brain tissue or brain cells is transduced in vivo. In some embodiments, a disease associated with the CNS is treated by administering one or more of the disclosed rAAV particles or compositions. In particular embodiments, a disease associated with glioma or glioblastoma is treated.

In some embodiments, “administering” or “administration” means providing a material to a subject in a manner that is pharmacologically useful. In some embodiments, a rAAV particle is administered to a subject enterally. In some embodiments, an enteral administration of the essential metal element/s is oral. In some embodiments, a rAAV particle is administered to the subject parenterally. In some embodiments, a rAAV particle is administered to a subject intratumorally, intraocularly, subcutaneously, intravenously (IV), intra-arterially, intracerebrally, intraventricularly, intramuscularly, intrathecally (IT), intracisternally, intraperitoneally, via inhalation, topically, or by direct injection to one or more cells, tissues, or organs (e.g., the brain). In certain embodiments, rAAV particle delivery is intratumoral.

In some embodiments, a subject in which a cell, tissue or organ is transduced is a vertebrate animal (e.g., a mammal or reptile). In some embodiments, a mammalian subject is a human, a non-human primate, a dog, a cat, a hamster, a mouse, a rat, a pig, a horse, a cow, a donkey or a rabbit. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, a subject is a model for a particular disease or used to study the pharmacokinetics and/or pharmacokinetics of a protein or siRNA encoded by a heterologous gene.

Sterile injectable solutions are prepared by incorporating the rAAV particles or preparations and/or compositions of modified cells, in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The amount of rAAV particle or preparation or modified cell compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the rAAV particle or preparation or composition, either over a relatively short, or a relatively prolonged period of time, as may be determined by the medical practitioner overseeing the administration of such compositions.

The composition may include rAAV particles or preparations or modified cell compositions, either alone, or in combination with one or more additional active ingredients, which may be obtained from natural or recombinant sources or chemically synthesized. In some embodiments, rAAV particles or preparations are administered in combination, either in the same composition or administered as part of the same treatment regimen, with a proteasome inhibitor, such as Bortezomib, or hydroxyurea.

In other aspects, provided herein are methods of treatment comprising administration of a chimeric receptor as described herein to a subject in need thereof. In certain embodiments, methods of treatment comprise administering a pharmaceutical composition, rAAV particle, or nanoparticle as described herein.

To “treat” a disease as the term is used herein, means to reduce the frequency or severity of at least one sign or symptom of a disease or disorder experienced by a subject. The compositions described above are typically administered to a subject in an effective amount, that is, an amount capable of producing a desirable result. The desirable result will depend upon the active agent being administered. For example, an effective amount of a rAAV particle may be an amount of the particle that is capable of transferring a heterologous nucleic acid to a host organ, tissue, or cell.

In certain embodiments, provided herein are methods of treating a subject having or at risk of developing a disease, disorder, or condition comprising administering to the subject a chimeric receptor or modified cell comprising a modified receptor as described herein. In other embodiments, the methods of treatment of a subject having or at risk of developing a disease, disorder, or condition comprise administering one or more pharmaceutical compositions, nanoparticles, or rAAV particles described herein.

In some embodiments, the subject has been diagnosed with a cancer. In some embodiments, the the subject has been diagnosed with a brain tumor. In particular embodiments, the subject has been disagnosed with glioma or GBM. In some embodiments, the subject has been diagnosed with a tumor or a non-brain organ that has metastasized to the brain.

Toxicity and efficacy of the compositions utilized in methods of the disclosure can be determined by standard pharmaceutical procedures, using either cells in culture or experimental animals to determine the LD50 (the dose lethal to 50% of the population). The dose ratio between toxicity and efficacy the therapeutic index and it can be expressed as the ratio LD50/ED50. Those compositions that exhibit large therapeutic indices are preferred. While those that exhibit a tolerable level of toxic side effects may be used, care should be taken to design a delivery system that minimizes the potential damage of such side effects. The dosage of compositions as described herein lies generally within a range that includes an ED50 with little or no toxicity. The dosage may vary within this range depending upon the dosage form employed and the route of administration utilized.

The amount of rAAV particle or preparation and time of administration of such particle or preparation will be within the purview of the skilled artisan having benefit of the present teachings. It is likely, however, that the administration of therapeutically-effective amounts of the rAAV particles or preparations of the present disclosure may be achieved by a single administration, such as for example, a single injection of sufficient numbers of infectious particles to provide therapeutic benefit to the patient undergoing such treatment. Alternatively, in some circumstances, it may be desirable to provide multiple or successive administrations of the rAAV particle or preparation, either over a relatively short, or a relatively prolonged period of time, as may be determined by the medical practitioner overseeing the administration of such compositions.

If desired, rAAV particles may be administered in combination with other agents, such as a checkpoint inhibitor (such as a PD-1 antagonist). In fact, there is virtually no limit to other components that may also be included, as long as the additional agents do not cause a significant adverse effect upon contact with the target cell (e.g., a cell of the nervous system) s or host tissues. The rAAV particles or preparations may thus be delivered along with various other pharmaceutically acceptable agents as required in the particular instance. Such compositions may be purified from host cells or other biological sources, or alternatively may be chemically synthesized as described herein.

In some embodiments, treatment of a subject with a rAAV particles as described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. In some embodiments, the disease, disorder, or symptom is cancer, such as a brain tumor. In some embodiments, the disease or symptom is SPG49 or HSAN9. In some embodiments, the symptom is intellectual disability, spastic ataxic gait, and autonomic dysfunction.

As is apparent to those skilled in the art in view of the teachings of this specification, an effective amount of viral vector to be added can be empirically determined. Administration can be administered in a single dose, a plurality of doses, continuously or intermittently throughout the course of treatment. Methods of determining the most effective means and dosages of administration are well known to those of skill in the art and will vary with the viral vector, the composition of the therapy, the target cell (e.g., a cell of the nervous system), and the subject being treated. Single and multiple administrations can be carried out with the dose level and pattern being selected by the treating physician.

Recombinant AAV (rAAV) Particles and Genomes

Aspects of the disclosure relate to recombinant adeno-associated virus (rAAV) particles or preparations of such particles for delivery of one or more polynucleotides or vectors comprising a sequence encoding a heterologous peptide, into various tissues, organs, and/or cells. In some embodiments, the rAAV particle is delivered to a host cell as described herein.

The wild-type AAV genome is a single-stranded deoxyribonucleic acid (ssDNA), either positive- or negative-sensed. The genome comprises two inverted terminal repeats (ITRs), one at each end of the DNA strand, two open reading frames (ORFs): rep and cap between the ITRs, and an insert nucleic acid positioned between the ITRs and optionally comprising a transgene. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ~2.3 kb- and a ~2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

Recombinant AAV (rAAV) particles may comprise a nucleic acid segment, which may comprise at a minimum: (a) one or more transgenes comprising a sequence encoding a heterologous peptide or an RNA of interest (e.g., a siRNA or microRNA) and (b) one or more regions comprising inverted terminal repeat (ITR) sequences (e.g., engineered ITR sequences) flanking the one or more heterologous nucleic acid regions (e.g., transgenes). In some embodiments, the ITRs are of the AAV2 serotype. In some embodiments, the nucleic acid segment is between 4 kb and 5 kb in size (e.g., 4.2 to 4.7 kb in size).

Any nucleic acid segment described herein may be encapsidated by a viral capsid, such as an AAV6 capsid or another serotype (e.g., a serotype that is of the same serotype as the ITR sequences), which may comprise a modified capsid protein as described herein. In some embodiments, the rAAV vector is pseudotyped with AAV2 ITRs and a modified AAV6 capsid (i.e., an rAAV2/6 vector or a variant thereof). In some embodiments, the modified capsid of any of the disclosed rAAV particles is an PHP.eB capsid. In some embodiments, the modified capsid is an rAAV6 (Y705F+Y731F+T492V) capsid (also known as a TM6 or AAV6-3pMut capsid). In some embodiments, provided herein are rAAV particles comprising any of the disclosed vectors, such as a vector containing an GFAP promoter, and further comprising an AAV6 capsid. In some embodiments, provided herein are rAAV particles comprising any of the disclosed vectors, such as a vector containing an GFAP promoter, and further comprising a TM6 capsid.

In some embodiments, the nucleic acid segment of the rAAV vector is circular. In some embodiments, the nucleic acid segment is single-stranded. In some embodiments, the nucleic acid segment is double-stranded. In some embodiments, a double-stranded nucleic acid segment may be, for example, a self-complimentary vector that contains a region of the nucleic acid segment that is complementary to another region of the nucleic acid segment, initiating the formation of the double-strandedness of the nucleic acid segment.

Accordingly, in some embodiments, an rAAV particle or rAAV preparation containing such particles comprises a viral capsid and a nucleic acid segment as described herein, which is encapsidated by the viral capsid. In some embodiments, the insert nucleic acid of the nucleic acid segment comprises (1) one or more transgenes comprising a sequence encoding a heterologous peptide, (2) one or more nucleic acid regions comprising a sequence that facilitates expression of the transgene (e.g., a promoter), and (3) one or more nucleic acid regions comprising a sequence that facilitate integration of the transgene (optionally with the one or more nucleic acid regions comprising a sequence that facilitates expression) into the genome of the subject. In certain embodiments, the promoter of the insert nucleic acid comprises a sequence that has at least 90%, at least 95%, or at least 99% identity to a chicken β-actin (CBA) promoter.

In some embodiments, the polynucleotides and vectors described herein comprise ITR sequences. In some embodiments, the coding sequence and associated promoter are flanked by rAAV ITR sequences. The ITR sequences of a polynucleotide described herein can be derived from any AAV serotype (e.g., 1, 2, 3, 4, 5, 6, 7, 8, 9, or 10) or can be derived from more than one serotype. In some embodiments, the ITR sequences are derived from AAV2 or AAV6. In some embodiments, the ITR sequences of the first serotype are derived from AAV3, AAV2 or AAV6. In other embodiments, the ITR sequences of the first serotype are derived from AAV1, AAV5, AAV8, AAV9 or AAV10. In some embodiments, the ITR sequences are the same serotype as the capsid (e.g., AAV3 ITR sequences and AAV3 capsid, etc.).

A non-limiting example of an ITR sequence (i.e., the AAV2 ITR sequence) useful in the disclosed rAAV vectors is provided below.

(SEQ ID NO: 7)
TTGGCCACTCCCTCTCTGCGCGCTCGCTCGCTCACTGAG
GCCGGGCGACCAAAGGTCGCCCGACGCCCGGGCTTTGCC
CGGGCGGCCTCAGTGAGCGAGCGAGCGCGCAGAGAGGGA
GTGGCCAACTCCATCACTAGGGGTTCCT

ITR sequences and plasmids containing ITR sequences are known in the art and commercially available (see, e.g., products and services available from Vector Biolabs, Philadelphia, PA; Cell Biolabs, San Diego, CA; Agilent Technologies, Santa Clara, Ca; and Addgene, Cambridge, MA; and Gene delivery to skeletal muscle results in sustained expression and systemic delivery of a therapeutic protein. Kessler P D, et al. Proc Natl Acad Sci USA. 1996; 93 (24): 14082-7; and Curtis A. Machida, Methods in Molecular Medicine™. Viral Vectors for Gene Therapy Methods and Protocols. 10.1385/1-59259-304-6:201 Humana Press Inc. 2003: Chapter 10, Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the nucleic acid segment comprises a pTR-UF-11 plasmid backbone, which is a plasmid that contains AAV2 ITRs. This plasmid is commercially available from the American Type Culture Collection (ATCC MBA-331).

The rAAV particle comprising a nucleic acid segment (in any form contemplated herein) may be delivered in the form of a composition, such as a composition comprising the active ingredient, such as the rAAV particle, nucleic acid segment (in any form contemplated herein), and a therapeutically or pharmaceutically acceptable carrier. The rAAV particles or nucleic acid segment may be prepared in a variety of compositions, and may also be formulated in appropriate pharmaceutical vehicles for administration to human or animal subjects.

Other aspects of the disclosure are directed to methods that involve contacting cells with an rAAV preparation produced by a method described herein. The contacting may be, e.g., ex vivo or in vivo by administering the rAAV preparation to a subject. The rAAV particle or preparation may be delivered in the form of a composition, such as a composition comprising the active ingredient, such as a rAAV particle or preparation described herein, and a therapeutically or pharmaceutically acceptable excipient. The rAAV particles or preparations may be prepared in a variety of compositions, and may also be formulated in appropriate pharmaceutical vehicles for administration to human or animal subjects.

The rAAV particle or particle within an rAAV preparation may be of any AAV serotype, including any derivative or pseudotype (e.g., 1, 2, 3, 4, 5, 6, 7, 8, 9, 10, 2/1, 2/5, 2/6, 2/8, 2/9, 3/1, 3/5, 3/8, or 3/9). As used herein, the serotype of an rAAV viral vector (e.g., an rAAV particle) refers to the serotype of the capsid proteins of the recombinant virus. In exemplary embodiments, the rAAV particle is of pseudotype rAAV2/6. In various embodiments, the AAV capsid is of serotype 6 (AAV6).

In some embodiments, the rAAV particle is not AAV2. In some embodiments, the rAAV particle is not AAV6. Non-limiting examples of derivatives and pseudotypes include rAAV.PHP.eB, AAV6 (TM6), AAV2 (Y444F+Y500F+Y730F) (i.e., TM2), rAAV2/1, rAAV2/5, rAAV2/6, rAAV2/8, rAAV2/9, AAV2-AAV3 hybrid, AAVrh.10, AAVrh.74, AAVhu.14, AAV3a/3b, AAVrh32.33, AAV-HSC15, AAV-HSC17, AAVhu.37, AAVrh.8, CHt-P6, AAV2.5, AAV6.2, AAV218, AAV-HSC15/17, AAVM41, AAV9.45, AAV6 (Y445F/Y731F), AAV2.5T, AAV-HAE1/2, AAV clone 32/83, AAVShH10, AAV2 (Y→F), AAV8 (Y733F), AAV2.15, AAV2.4, AAVM41, AAV-DJ, and AAVr3.45. The AAV6 (TM6) (also known as AAV6-3pmut and AAV6 (Y705F+Y731F+T492V)) capsid is described in Rosario et al., Microglia-specific targeting by novel capsid-modified AAV6 vectors, Mol Ther Methods Clin Dev. 2016; 13; 3:16026 and International Patent Publication No. PCT/US 2016/126857, each of which are herein incorporated by reference.

These AAV serotypes and derivatives/pseudotypes, and methods of producing such derivatives/pseudotypes are known in the art (see, e.g., Mol Ther. 2012 April;20 (4): 699-708). The AAV vector toolkit: poised at the clinical crossroads. Asokan A1, Schaffer DV, Samulski R J.). In some embodiments, the rAAV particle is a pseudotyped rAAV particle, which comprises (a) a nucleic acid segment comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Additional capsids suitable for use in the disclosed rAAV particles include capsids comprising non-native amino acid substitutions at amino acid residues of a wild-type AAV6 capsid, wherein the non-native amino acid substitutions comprise one or more of Y445F, Y705F, Y731F, T492V and S663V.

Additional serotypes of the rAAV capsids disclosed herein include capsids include AAV7m8, AAV2/2-MAX, AAVSHh10Y, AAV3b, AAVLK03, AAV7BP2, AAV1 (E531K), AAV6 (D532N), and AAV2G9.

The AAVSHh10 and AAV6 (D532N) capsids, both derivatives of AAV6, are described in Klimczak et al. (2009) A Novel Adeno-Associated Viral Variant for Efficient and Selective Intravitreal Transduction of Rat Muller Cells. PLOS ONE 4 (10): e746, herein incorporated by reference.

rAAV Production Methods

Methods of producing rAAV particles are described herein. Other methods are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Nos. US 2007/0015238 and US 2012/0322861, which are incorporated herein by reference; and plasmids and kits available from ATCC and Cell Biolabs, Inc.). For example, a plasmid containing the nucleic acid segment may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

In some embodiments, the one or more helper plasmids include a first helper plasmid comprising a rep gene and a cap gene and a second helper plasmid comprising a Ela gene, a Elb gene, a E4 gene, a E2a gene, and a VA gene. In some embodiments, the rep gene is a rep gene derived from AAV3, AAV5, or AAV6 and the cap gene is derived from AAV2, AAV3, AAV5, or AAV6 and may include modifications to the gene in order to produce the modified capsid protein described herein. In some embodiments, the rep gene is a rep gene derived from AAV1 or AAV2 and the cap gene is derived from AAV1 or AAV2 and may include modifications to the gene in order to produce the modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG (R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, Ca; and Addgene, Cambridge, MA; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R.O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. In some embodiments, the one or more helper plasmids comprise rep genes for a first serotype (e.g., AAV3, AAV5, and AAV6), cap genes (which may or may not be of the first serotype) and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid segment described herein. The HEK293 cells are then incubated for at least 60 hours to allow for rAAV particle production. Alternatively, in another example Sf9-based producer stable cell lines are infected with a single recombinant baculovirus containing the nucleic acid segment. As a further alternative, in another example HEK293 or BHK cell lines are infected with a HSV containing the nucleic acid segment and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

### Host Cells

The disclosure also contemplates modified host cells that comprise at least one of the disclosed rAAV particles or expression vectors described herein. Such host cells include mammalian host cells, with human host cells being preferred, and may be either isolated, in cell or tissue culture. In the case of genetically modified animal models (e.g., a mouse), the transformed host cells may be comprised within the body of the animal itself.

The disclosure also contemplates host cells that comprise at least one of the disclosed rAAV particles or vectors. Such host cells include mammalian host cells, with human host cells being preferred, and may be either isolated, in cell or tissue culture. In the case of genetically modified animal models (e.g., a mouse), the transformed host cells may be comprised within the body of a non-human animal itself. Human host cells are contemplated.

In some embodiments, the modified host cells are astrocytes. In some embodiments, the host cells are microglial cells. In some embodiments, the host cell is a monocyte-like (or macrophage-like) RAW264.7 cell. In some embodiments, the host cell is a cancer cell, such as an HEK293 cell or an H4 (human neuroglioma) cell.

In some embodiments, a host cell as described herein is derived from a subject as described herein. Host cells may be derived using any method known in the art, e.g., by isolating cells from a fluid or tissue of the subject. In some embodiments, the host cells are cultured. Methods for isolating and culturing cells are well known in the art.

Non-limiting examples of subjects include humans, dogs, cats, mice, rats, and transgenic species thereof. Preferably, the subject is a human. The cells can be obtained from a number of sources, including peripheral blood mononuclear cells, bone marrow, lymph node tissue, spleen tissue, umbilical cord, and tumors. In certain embodiments, any number of monocyte or progenitor cell lines available in the art, may be used. In certain embodiments, the cells can be obtained from a unit of blood collected from a subject using any number of techniques known to the skilled artisan. In some embodiments, cells from the circulating blood of an individual are obtained by apheresis or leukapheresis.

In some embodiments, a population or plurality of cells comprises the monocytes, astrocytes, or microglial cells of the present disclosure. Examples of a population of cells include, but are not limited to, a purified population of microglia, monocytes or astrocytes, and a cell line. In another embodiment, peripheral blood mononuclear cells comprise the population of monocytes. In yet another embodiment, purified cells comprise the population of monocytes.

Provided herein are pharmaceutical compositions that comprise a modified cell as disclosed herein. These modified cells may comprise astrocytes, microglial cells, or monocytes. In exemplary embodiments, provided herein are pharmaceutical compositions comprising modified astrocytes. These modified cells may be contacted ex vivo with a nucleic acid or expression vector expressing one of the disclosed CPRs. In some embodiments, the expression vector is a viral vector, such as an rAAV vector or a lentiviral vector. In particular embodiments, the expression vector is an rAAV vector.

In some aspects, ex vivo delivery of cells transduced with rAAV particles or preparations is provided herein. Ex vivo gene delivery may be used to transplant rAAV-transduced host cells that were isolated from the host back into the host. A suitable ex vivo protocol may include several steps. For example, a segment of target tissue or an aliquot of target fluid may be harvested from the host and rAAV particles or preparations may be used to transduce a polynucleotide into the host cells in the tissue or fluid. These genetically modified cells may then be transplanted back into the host. Several approaches may be used for the reintroduction of cells into the host, including intravenous injection, intracranial injection, ICV injection, or in situ injection into target tissue. Autologous and allogeneic cell transplantation may be used according to the disclosure.

An effective amount of cells in a pharmaceutical composition for ex vivo delivery to a subject is at least one cell (for example, one modified astrocyte cell), or is more typically greater than 100 cells, for example, up to 106, up to 107, up to 108 cells, up to 109 cells, up to 1010 cells, or up to 1011 cells or more. In certain embodiments, the cells are administered in a range from about 106 to about 1010 cells/m². The number of cells will depend upon the ultimate use for which the composition is intended, as well the type of cells included therein. For example, a composition comprising cells modified to contain a CPR specific for a particular neurodegenerative disease antigen (e.g., amyloid beta) will comprise a cell population containing from about 5% to about 95% or more of such cells. In certain embodiments, a composition comprising CPR modified cells comprises a cell population comprising at least 5%, 10%, 15%, 20%, 25%, 30%, 35%, 40%, 45%, 50%, 55%, 60%, 65%, 70%, 75%, 80%, 85%, 90%, 95% or more of such cells.

### Kits

Herein are described compositions including one or more of the disclosed rAAV vectors comprised within a kit for diagnosing, preventing, treating or ameliorating one or more symptoms of a cancer, such as a brain tumor. Such kits may be useful in the diagnosis, prophylaxis, and/or therapy or a human disease, and may be particularly useful in the treatment, prevention, and/or amelioration of one or more symptoms of GBM or glioma.

Kits comprising one or more of the disclosed rAAV vectors (as well as one or more virions, viral particles, transformed host cells or pharmaceutical compositions comprising such vectors); and instructions for using such kits in one or more therapeutic, diagnostic, and/or prophylactic clinical embodiments are also provided according to several embodiments. Such kits may comprise one or more reagents, restriction enzymes, peptides, therapeutics, pharmaceutical compounds, or means for delivery of the composition(s) to host cells, or to an animal (e.g., syringes, injectables, and the like). Exemplary host cells of the disclosure include HEK293 and C2C12 myoblast cells. Additional exemplary host cells of the disclosure include human cells derived from an induced pluripotent stem cell or a neuronal cell line. Depending on the embodiment, kits include those for treating, preventing, or ameliorating the symptoms of a disease, deficiency, dysfunction, and/or injury, or may include components for the large-scale production of the viral vectors themselves.

In some embodiments, a kit comprises one or more containers or receptacles comprising one or more doses of any of the described therapeutic. Such kits may be therapeutic in nature. In some embodiments, the kit contains a unit dosage, meaning a predetermined amount of a composition comprising, e.g., a described therapeutic with or without one or more additional agents.

One or more of the components of a kit can be provided in one or more liquid or frozen solvents. The solvent can be aqueous or non-aqueous. The formulation in the kit can also be provided as dried powder(s) or in lyophilized form that can be reconstituted upon addition of an appropriate solvent.

In some embodiments, a kit comprises a label, marker, package insert, bar code and/or reader indicating directions of suitable usage of the kit contents. In some embodiments, the kit may comprise a label, marker, package insert, bar code and/or reader indicating that the kit contents may be administered in accordance with a certain dosage or dosing regimen to treat a subject.

In addition, a kit may also contain various reagents, including, but not limited to, wash reagents, elution reagents, and concentration reagents. Such reagents may be readily selected from among the reagents described herein, and from among conventional concentration reagents.

As used herein, the term “kit” may be used to describe variations of the portable, self-contained enclosure that includes at least one set of components to conduct one or more of the diagnostic or therapeutic methods of the disclosure.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus may be considered to constitute preferred modes for its practice. However, those of skill in the art should, in light of the present disclosure, appreciate that many changes may be made in the specific embodiments which are disclosed and still obtain a like or similar result without departing from the spirit and scope of the invention.

### Example 1

### Chemokine analysis of Human GBM favors MDSC recruitment over lymphocyte infiltration

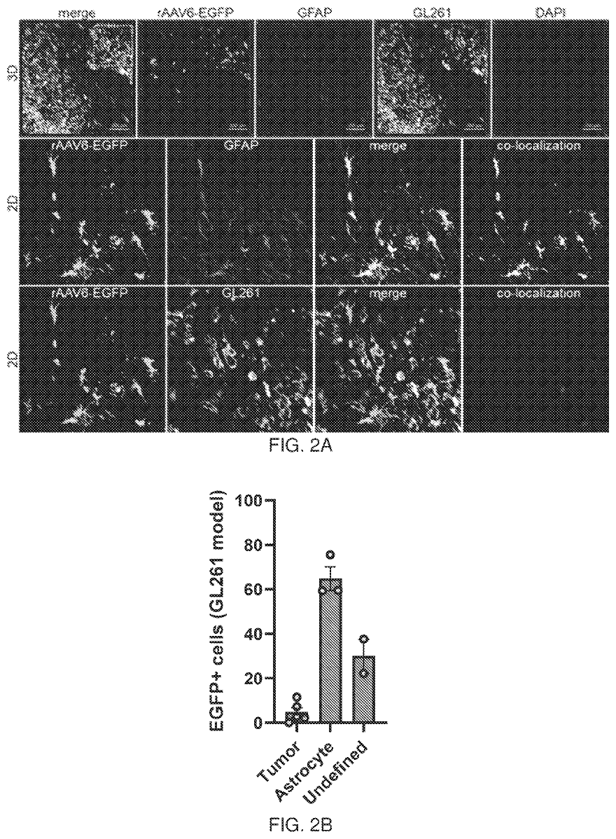

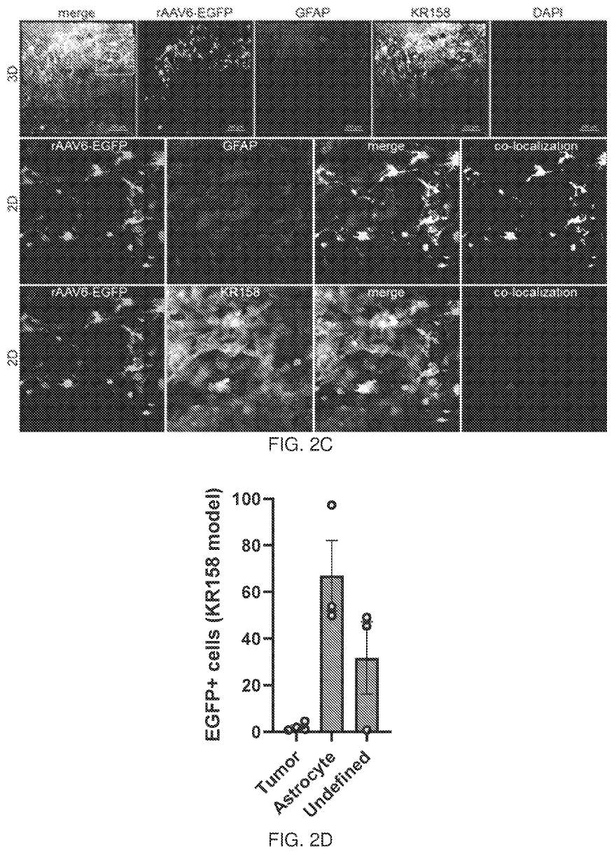

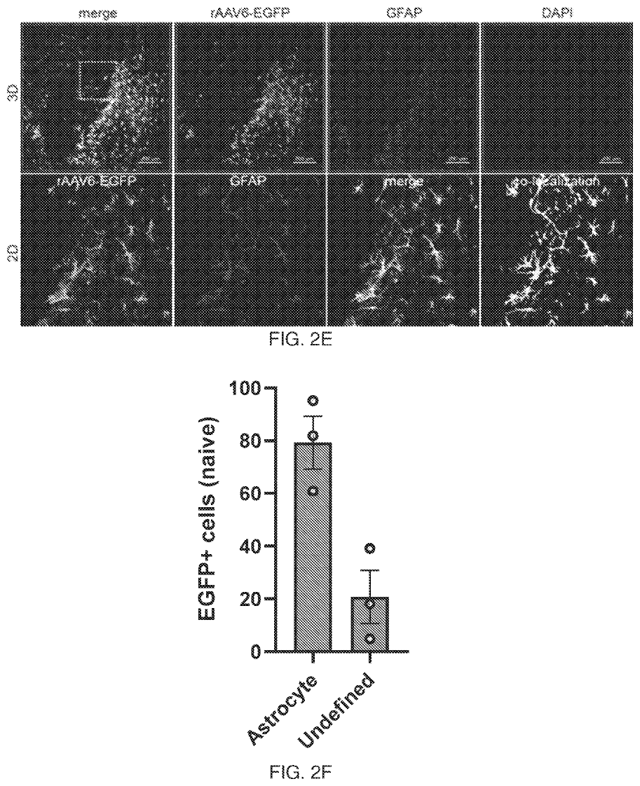

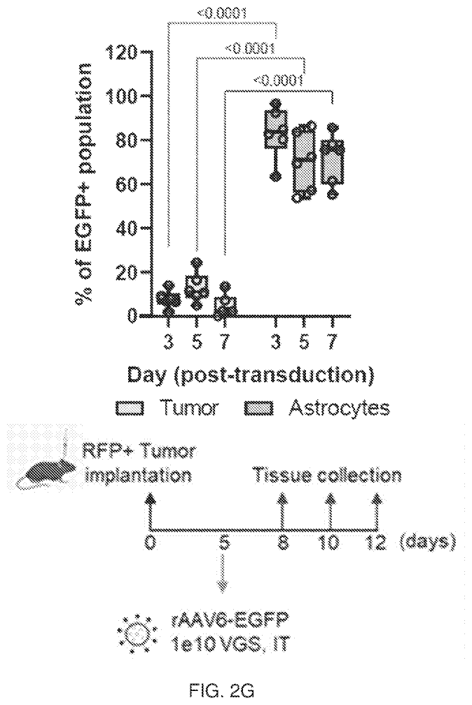

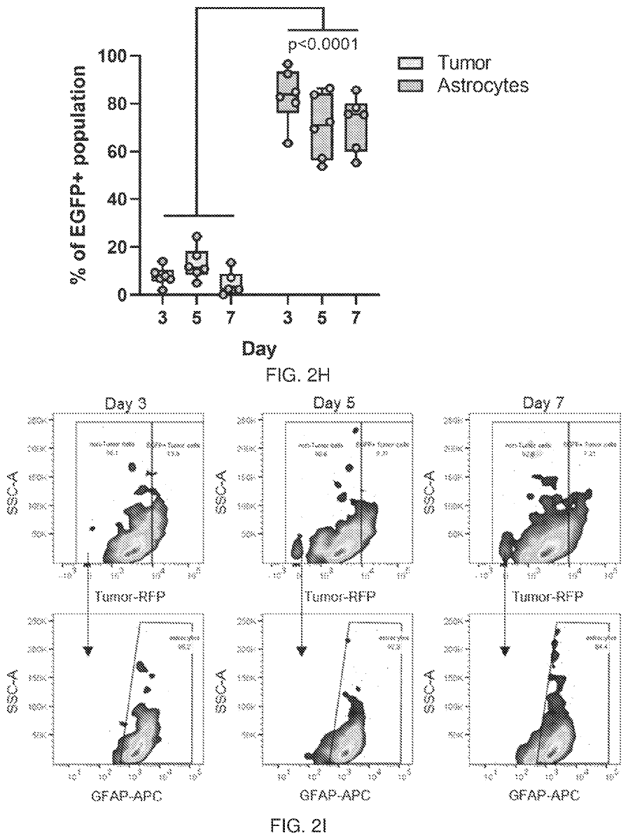

Upon examination of human glioma tumors for CD3 protein expression provided through the Human Protein Atlas, it was determined that over 80% of tumor specimens are negative for lymphocyte infiltrates (FIGS. 1C-1D), corroborating prior literature describing the lymphocyte-replete nature of these tumors (Rahman 2020 Adult immuno-oncology NEUROONC). To determine if a lack of lymphocyte chemotactic factors was a possible reason for the cold nature of these tumors, human glioma samples were screened for the expression of 31 chemokines. Of these, CXCL4, CXCL7, CXCL8 (IL-8), CXCL16, LCF (IL-16), TIG-2, and midkine emerged as the most abundant secreted chemokines detected (FIGS. 2A, 2E), where these ligands likely play a significant role in the recruitment of myeloid-derived suppressor cells in the context of gliomagenesis. Notably, chemotactic factors that favor lymphocyte recruitment were poorly expressed, including CXCL9 (MIG), and MIP-1α/β (CCL3/CCL4) (FIGS. 2A, 2E). To that end, it was possible that rAAV delivery and restoration of “call-and-receive” signals for T lymphocytes within the TME would enhance their recruitment to the tumor. Pro-inflammatory chemokine (C—X—C motif) ligand 9 (CXCL9) was selected as the lead candidate, as CXCL9 is a powerful attractant known to induce the migration, differentiation, and activation of cytotoxic T cells. CXCL9 expression in many tumors correlated with anti-tumor immune activity and is shown to be predictive of response to checkpoint blockade. Intra-tumor delivery of AAV encoded CXCL9 resulted in the production of a pro-lymphocyte chemotactic gradient by transduced tumor and/or tumor microenvironment (e.g. reactive astrocytes, microglia, infiltrating myeloid cells, etc.) that increased tumor infiltration by lymphocytes possessing the cognate receptor for CXCL9-CXC motif chemokine receptor 3 (CXCR3) as briefly summarized in FIG. 1E. This augmented the efficacy of T lymphocyte directed anti-tumor therapeutic strategies, such as immune checkpoint blockade or adoptive cellular transfer (ACT).

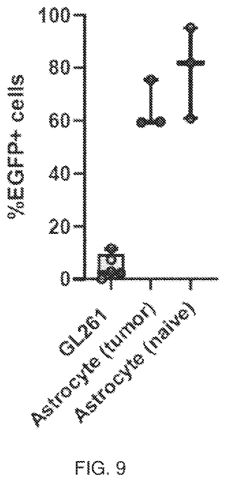

Historically, targeted transduction with rAAV has proven challenging despite elegant efforts in capsid evolution studies and capsid engineering. To achieve sufficient transgene expression, transduction of either tumor cells or tumor-associated stroma is likely necessary, where intra-tumoral delivery would minimize the potential for systemic toxicities and decrease off-target homing of T cells. To identify an appropriate rAAV capsid for targeting glioma tumors, an in vitro capsid screen in 15 unique glioma cell models consisting of patient derived and murine xenografts was performed. Enhanced green fluorescent protein (EGFP) was encoded into a rAAV2 single stranded vector, utilizing the non-cell-autonomous, constitutively active CBA promotor to drive transgene expression. These constructs were then pseudotyped into each of 29 unique capsids as previously described (Goodwin 2020 Utilizing minimally MOL DEGENERATION; O'Carroll, Young AAV targeting of glial cell types 2021 FRON MOL NEURO). Transduction in each of the 15 unique glioma cell models, including primary human and murine xenografts, was assessed via EGFP relative fluorescence. Based on the results of this screen, rAAV6 was selected for further examination as it demonstrates moderate to high transduction in nearly all models tested. Quantitative flow cytometric evaluation of EGFP expression in three distinct models of aggressive syngeneic murine glioblastoma 72 hours following rAAV6-EGFP transduction shows moderate transduction of GL261 and KR158, with >25% of cells positive for the transgene at this time point, with lower transduction observed in CT-2A cells (<20%). An AAV6 capsid encoding murine CXCL9 in an AAV2 vector (rAAV6-CXCL9) was generated to examine the effects of this construct on immune trafficking in syngeneic murine glioblastoma (FIG. 9).

### AAV6 Transduces Tumor-Reactive Astrocytes In Vivo in Preclinical Models of GBM

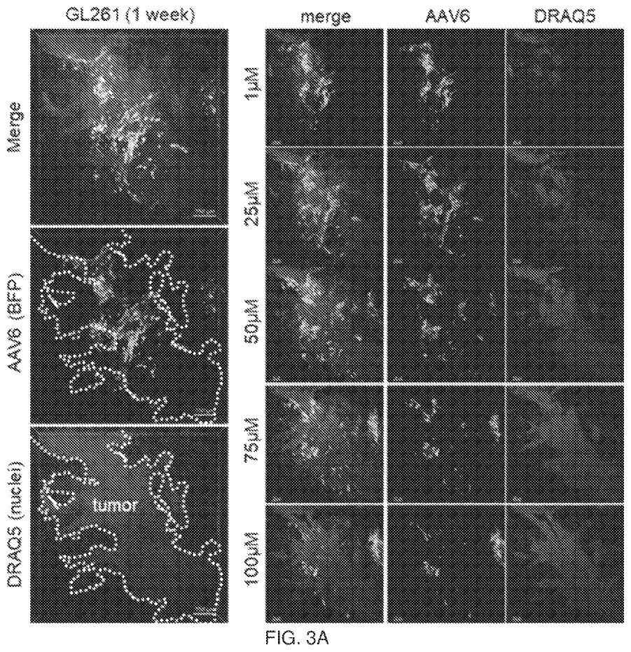

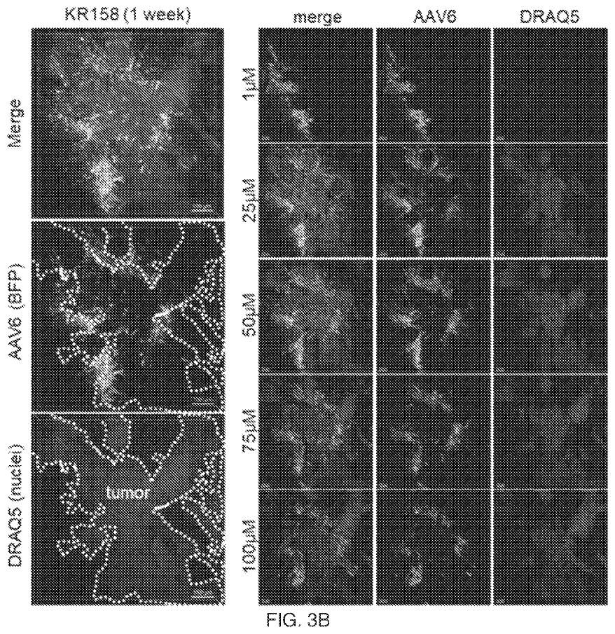

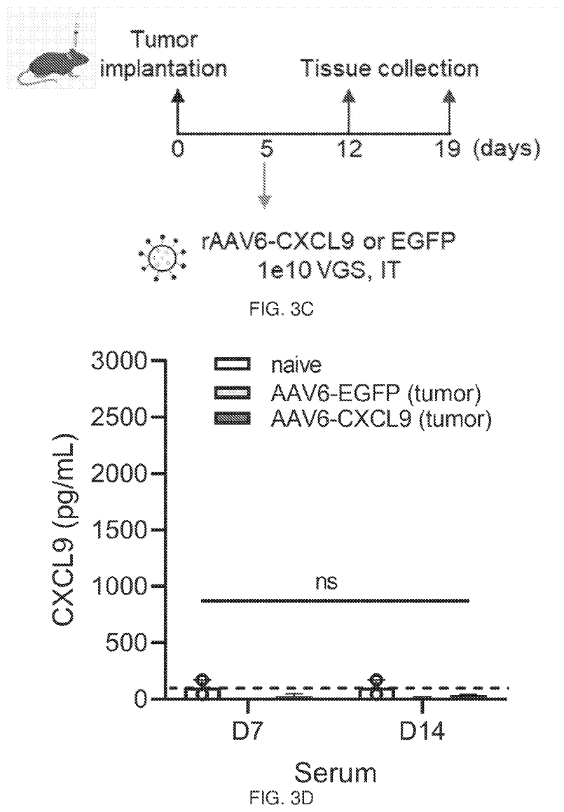

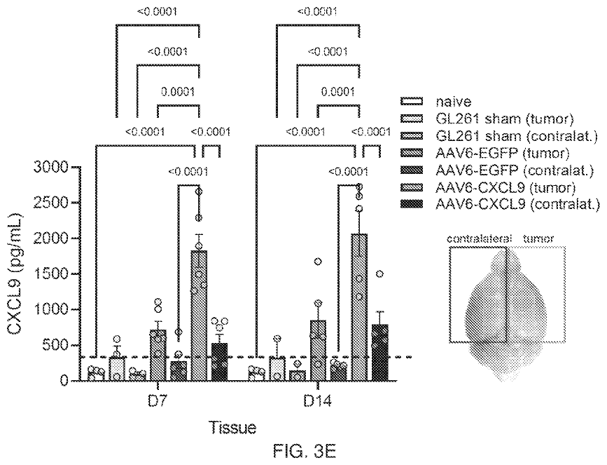

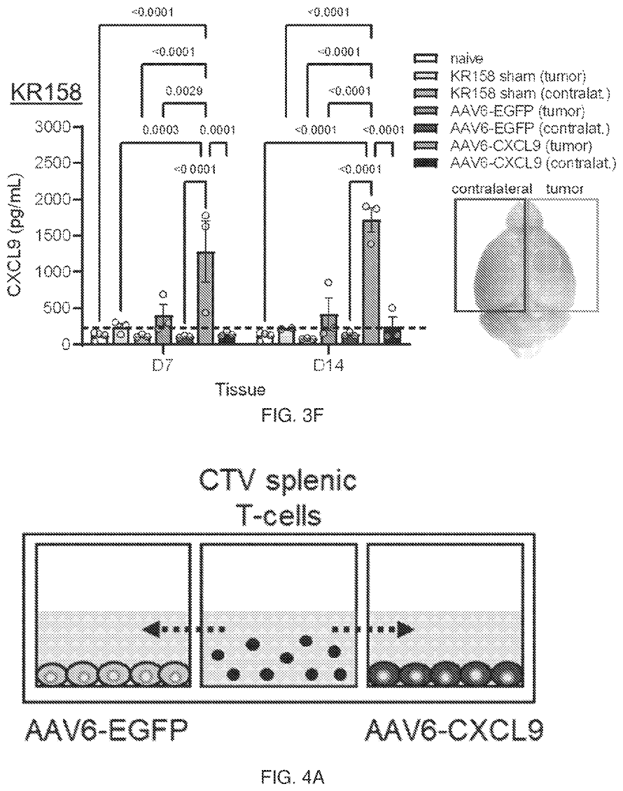

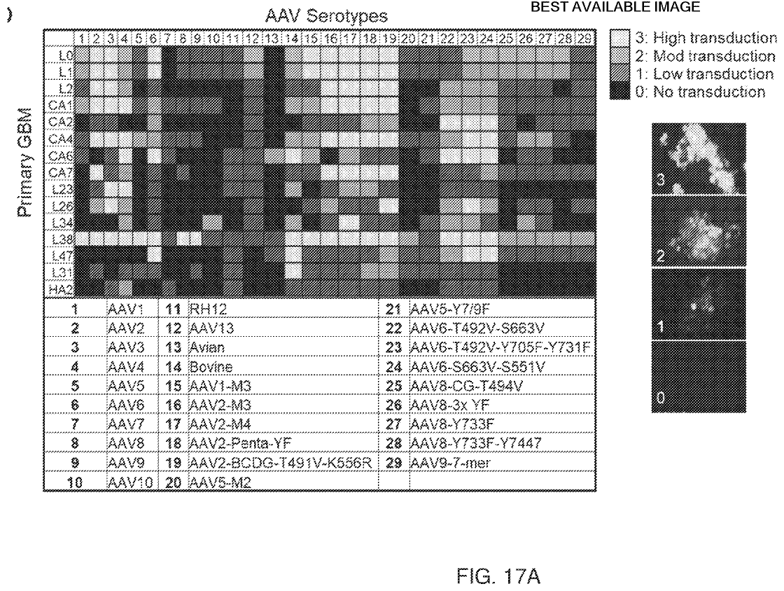

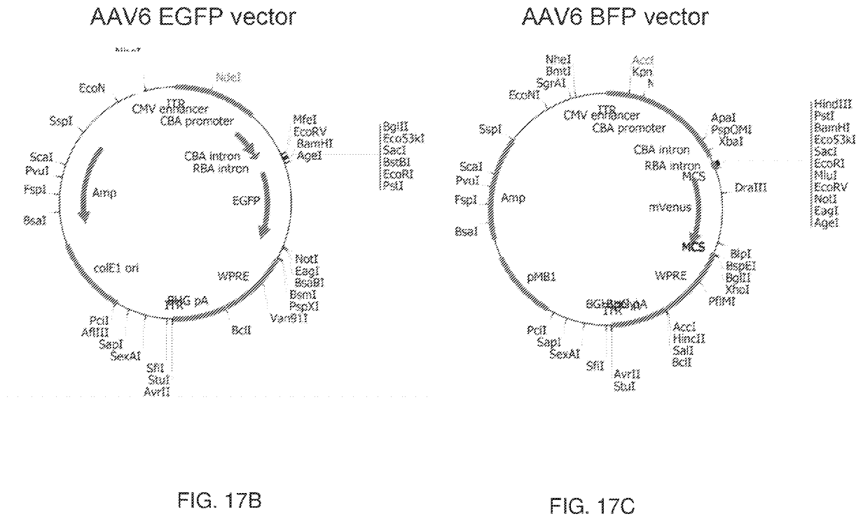

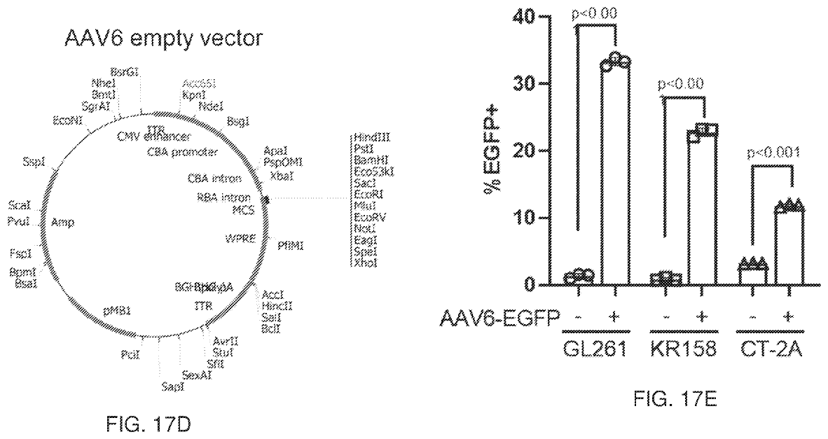

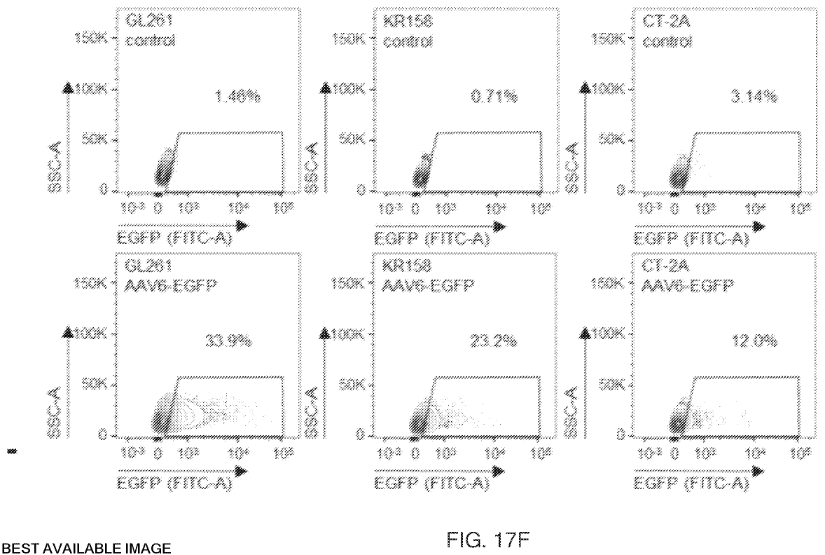

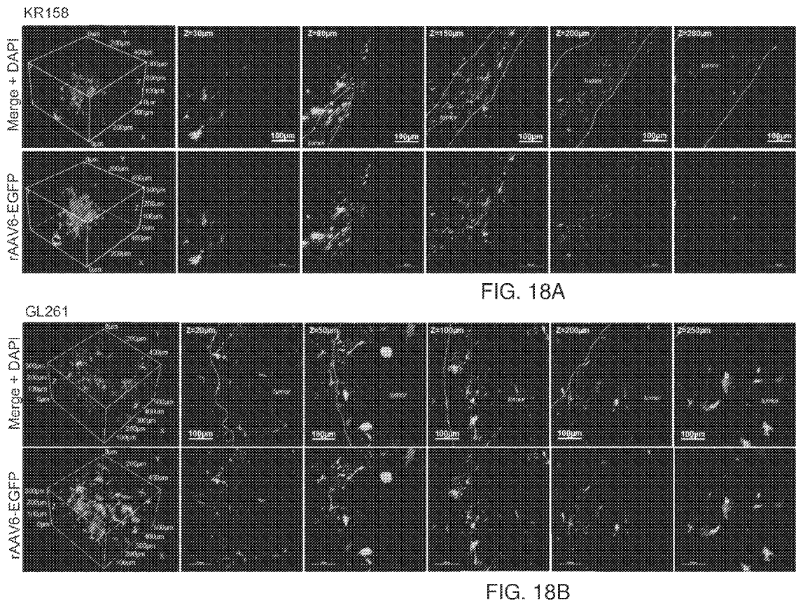

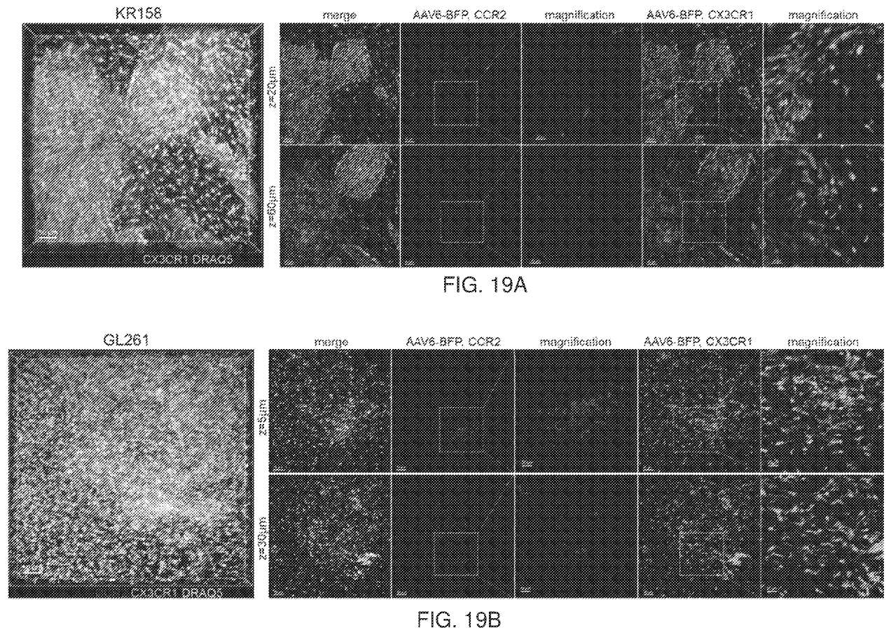

To define AAV6 tropism in murine GBM in vivo, AAV6-EGFP (FIG. 17B) was intratumorally injected into established intracranial KR158 and GL261 tumors. EGFP expression was detected 1 week following tumor transduction in both models (FIGS. 18A-18B), however the morphological appearance and contiguous distribution of transduced cells suggest that AAV6 targeted cells are likely tumor-associated, and not cancer cells directly. Microglia and tumor-associated macrophages are reported to comprise a significant cellular proportion of glioma tumors, and so it may be beneficial to identify if AAV6 was targeting either population. KR158 and GL261 were implanted into CCR2RFPCX3CR1GFP (B6.129 (Cg)-Cx3cr1tm1Litt Ccr2tm2.1lfc/JernJ) dual reporter mice, where microglia can be identified via GFP expression, and bone marrow-derived inflammatory cells via RFP expression. Tumors were evaluated by 3D IHC for viral transduction one week following intratumor injection with AAV6 encoding a BFP reporter (FIG. 17C). In both tumor model systems no co-localization between BFP and either RFP or GFP was observed, indicating that neither microglia nor tumor-associated macrophages are the principal target of AAV6 transduction (FIGS. 19A-19B). To assess the degree of AAV6 transduction specifically in tumor cells, RFP-labeled KR158 or GL261 cells were implanted in mice. Tumors were evaluated by 3D IHC for viral transduction one week following intratumor injection with AAV6-EGFP. Resected tumors were immuno-labeled against glial fibrillary acidic protein (GFAP) to detect astrocytes, another candidate tumor-associated cell population. Both tumor models reveal a high degree of overlap between GFAP (red pseudocolor) and EGFP (green pseudocolor), with minimal overlap between tumor cells (light blue pseudocolor) and EGFP (FIGS. 2A, 2C), indicating that EGFP-positive cells are likely astrocytes. Voxel-based co-localization algorithms to quantitate EGFP co-localization with each tumor or astrocytes confirm astrocytes as the principal cell target of AAV6 transduction, accounting for ~60-70% of EGFP-positive cells in both GL261 (FIG. 2E) and KR158 (FIG. 3D) intracranial tumors. Because tumor presence can stimulate different activation states in astrocytes that may cause them to be more or less susceptible to viral transduction, CNS tropism of AAV6 was also evaluated in age-matched naïve mice. AAV6 was equally efficient at transducing astrocytes in naïve animals as shown by co-localization between GFAP immunostaining and EGFP transgene expression (FIGS. 2E, 9).

One of the advantageous features of AAV gene transduction is that it rarely integrates into the host genome. Following uncoating in the host nucleus, single-stranded genomes are converted to double-stranded multimeric circular concatemeric episomes. As such, AAV transgene expression can persist long-term in post mitotic cells. Because tumor cells undergo rapid cell division, it may be possible that transgene expression is lost over time through sequential dilution of episomes passed down to daughter cells. To explore this, AAV6-EGFP transgene expression was evaluated longitudinally across early time points in mice harboring RFP labeled GL261 cells, which demonstrate the highest transduction efficiency in vitro (FIGS. 17E-17F). Tumors were resected 3, 5, and 7 days following AAV6-EGFP intra-tumor injection as outlined in FIG. 2G, and EGFP transgene expression in tumors and astrocytes was measured by flow cytometry. Even at early time points, AAV6 predominantly transduces astrocytes identified as GFAP+RFP− (70-80% EGFP+ cells), with limited EGFP expression observed in RFP+ tumor cells (<15%) (FIG. 2G, FIG. 20A). By 7 days post intratumor viral injection, less than 5% of EGFP+ cells on average were RFP+ tumor cells, where astrocytes consistently comprised 70-80% of EGFP+ cells at each time point. These data indicate that AAV6 more selectively transduces astrocytes in vivo, with limited and transient expression in tumor cells.

### Transgene Signal Distribution and Durability in Glioblastoma

Next, the distribution of transgene signal was examined in both the GL261 and KR158 tumor models via 3D IHC to better understand the avidity of AAV6 for tumor-associated versus distal astrocytes following direct intra-tumor injection. BFP transgene expression was observed in a peritumoral pattern in and around the tumor body in both model systems (FIG. 3A, 3B), redolent of glial scar formation found in human brain malignancies. As CXCL9 is a small, secreted chemokine, it may be beneficial to determine if signal expression was still focal to the tumor or could be detected in contralateral brain and/or systemically. Whole brain was collected at 1 and 2 weeks following AAV6-CXCL9 or AAV6-EGFP intratumor injection as outlined in FIG. 3C. Cerebellar tissue was removed, and remaining tissue was dissected into the tumor containing and contralateral hemispheres. Serum was collected following peripheral blood draws taken from the posterior vena cava. Brain tissue and serum was also collected from non-transduced (sham) tumor controls, and naïve (non-tumor bearing) controls to establish CXCL9 baseline values. Serum levels of CXCL9 following intratumor delivery of AAV6-CXCL9 measured using high sensitivity ELISA assay did not exceed those observed in naïve controls (FIGS. 3B, 3D). In the brain, elevated CXCL9 expression was selectively detected in the tumor bearing hemisphere transduced with AAV-CXCL9, with minimal signal observed in the contralateral hemisphere in both GL261 and KR158 model systems (FIG. 3E-3F). Transgene CXCL9 expression appears to be stable, as signal intensity was consistent in AAV6-CXCL9 transduced tumors at both the 1 and 2 week time points in each tumor model (FIG. 3E-3F). Of note, a small increase in CXCL9 expression was observed in AAV6-EGFP control transduced GL261 tumors and could be indicative of a mild inflammatory response to AAV6, however these values were not found to be statistically significant. Together these data demonstrate that AAV6 intratumor delivery of CXCL9 results in focal and durable expression of encoded transgene, where tumor-reactive astrocytes are the target of AAV6 transduction.

### Effects of AAV6-CXCL9 on Lymphocyte Chemotaxis

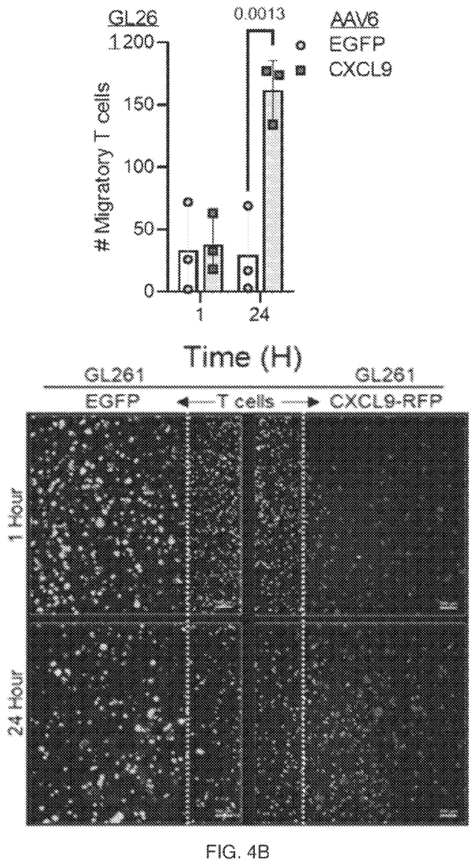

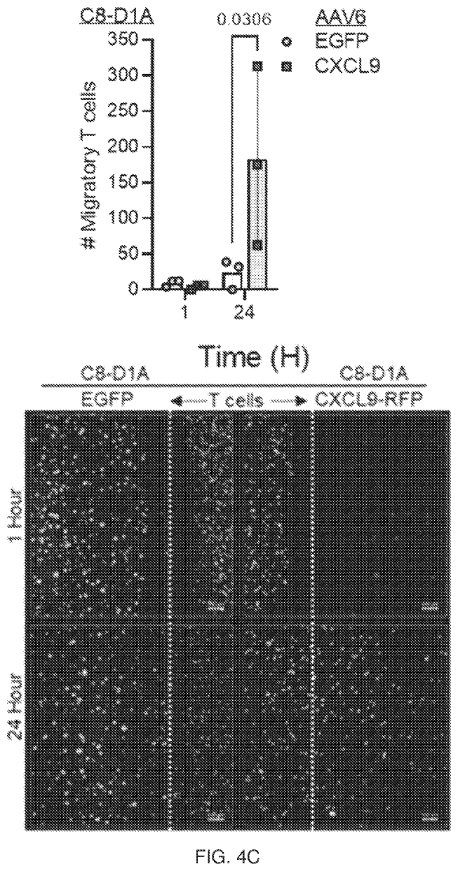

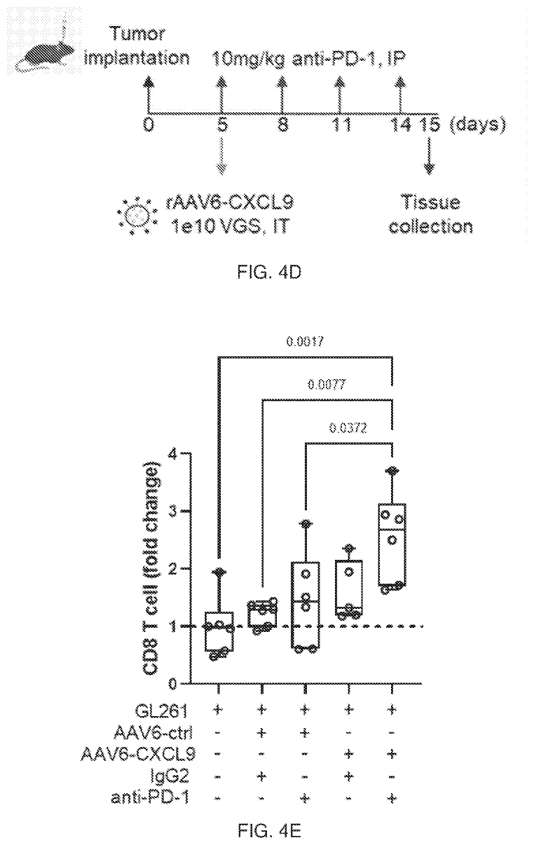

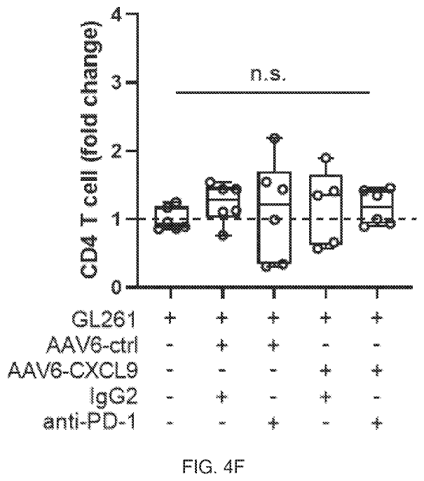

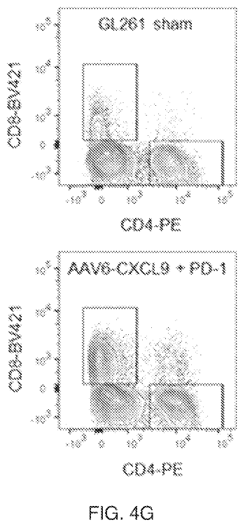

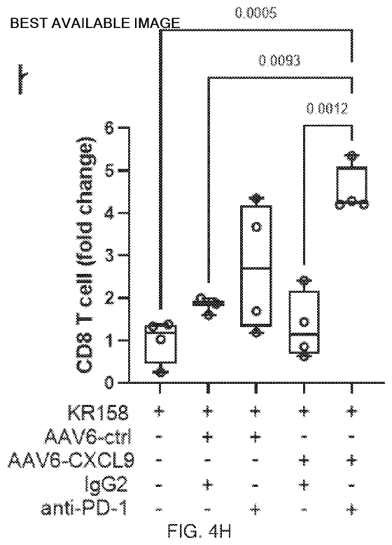

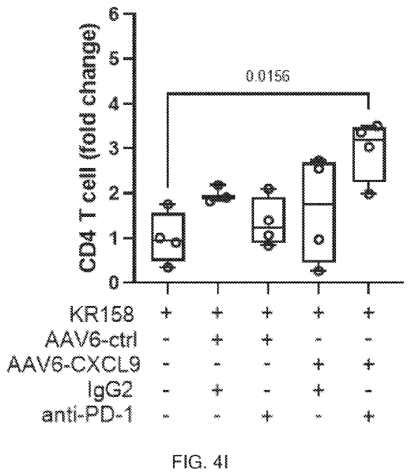

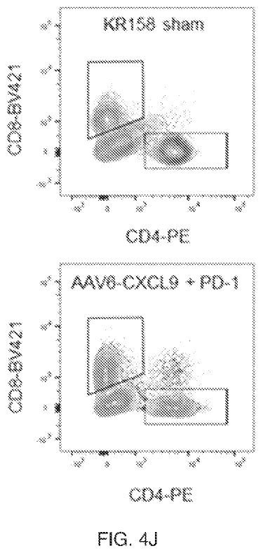

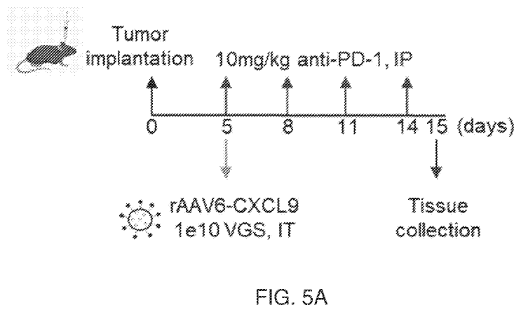

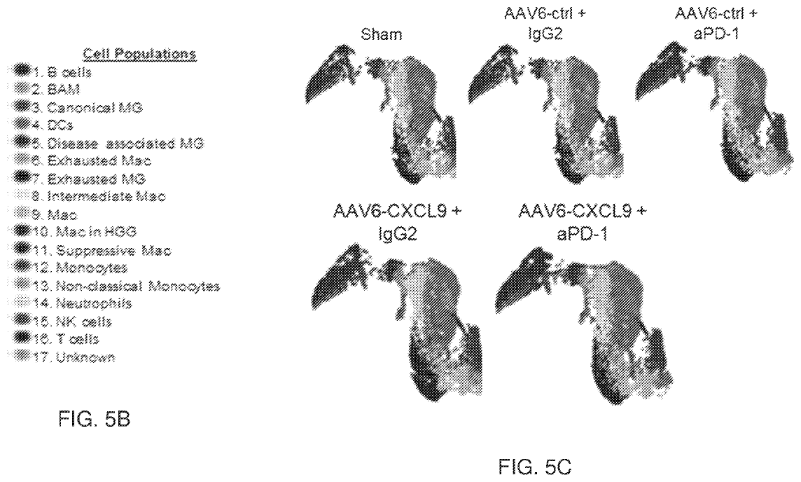

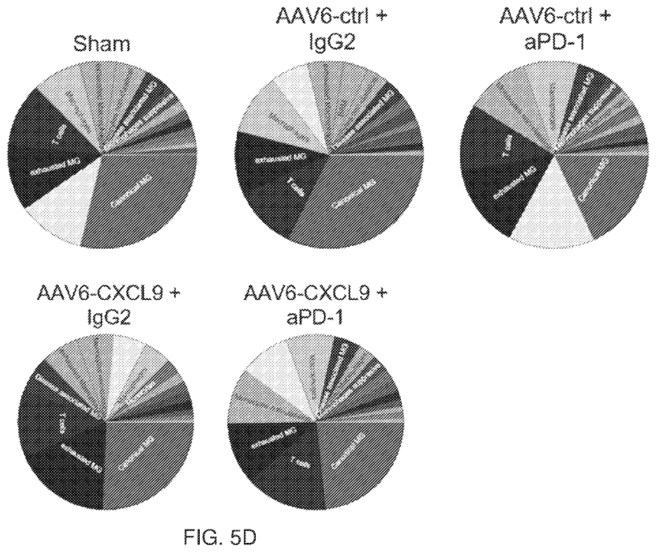

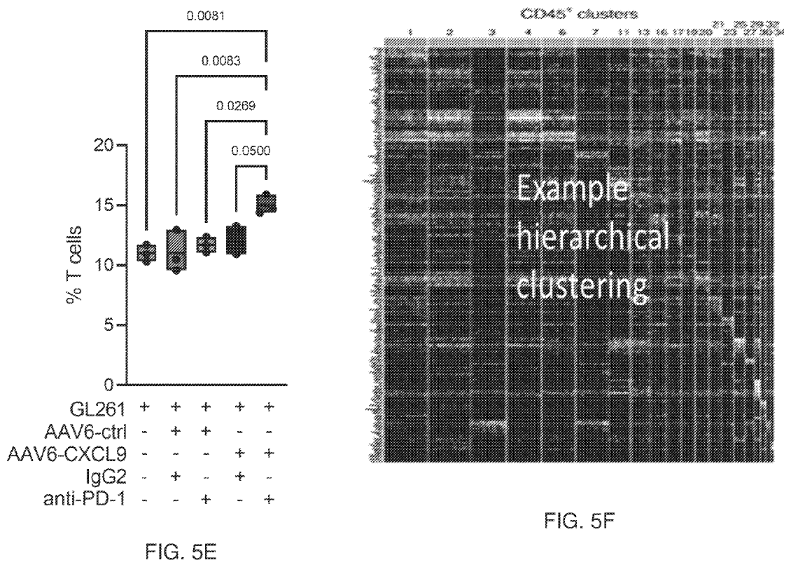

To evaluate the biologic activity of AAV6-CXCL9 on lymphocyte recruitment, competitive in vitro chemotaxis assays were performed. Briefly, CTV-labeled splenic-derived T lymphocytes were flanked by target cells transduced with AAV6 encoding either EGFP or CXCL9, and migration was monitored via fluorescence microscopy at 1 and 24 hours following co-culture (FIG. 4A). Using GL261 tumor cells as the target population for AAV6 transduction, significantly more T lymphocytes co-localized in the CXCL9 transduced tumor field as compared to EGFP at 24 hours (FIG. 4B). Given that astrocytes are the principle target of AAV6 transduction in vivo, chemotaxis was reassessed via competitive co-culture using astrocytes (C8-D1A) in lieu of GL261 glioma cells. Astrocytes transduced with AAV6-CXCL9 similarly showed enhanced recruitment of T lymphocytes (FIG. 4C), confirming that transgene encoding CXCL9 produces a biologically functional chemokine. To determine the effect of AAV6-CXCL9 on T lymphocyte recruitment in vivo, multiparametric flow cytometry was performed to quantitate the number of T cells present in dissociated tumors following intratumor delivery. These studies were done in combination with anti-PD-1 ICB, where tissue was collected one day following the final dose of ICB to capture events within therapeutic response window as outlined in FIG. 4D. In both GL261 and KR158 tumor models AAV6-CXCL9 alone had minimal impact on enhancing T cell recruitment to the tumor, however significant increases in T lymphocyte infiltration was observed in the context of combination treatment. AAV6-CXCL9 plus ICB increased CD8 T lymphocytes >2.5-fold in the GL261 model and >4.5-fold in the KR158 model (FIGS. 4E-4F and FIG. 20B). While no significant changes in CD4 T lymphocyte recruitment in response to treatment was observed in the GL261 model (FIG. 3H), a >3-fold increase was detected in the KR158 model (FIG. 3I). Anti-PD-1 ICB treatment in combination with control AAV6 (EGFP) modestly increased CD8 T lymphocyte recruitment in GL261 by 1.4-fold and in KR158 by 2.7-fold, indicating that CXCL9 markedly improves tumor infiltration by these cells. These data highlight a potential role for anti-PD-1 ICB in mobilizing T lymphocytes systemically, where sequestration of T lymphocytes was recently proposed as a novel mechanism of immune suppression in brain tumors.

scRNAseq Identifies Treatment-Related Immune Response to AAV6-CXCL9 and Anti-PD-1 ICB

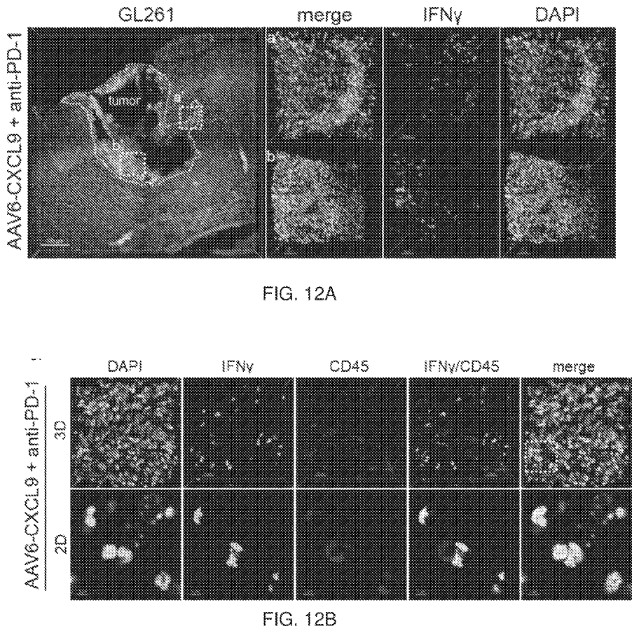

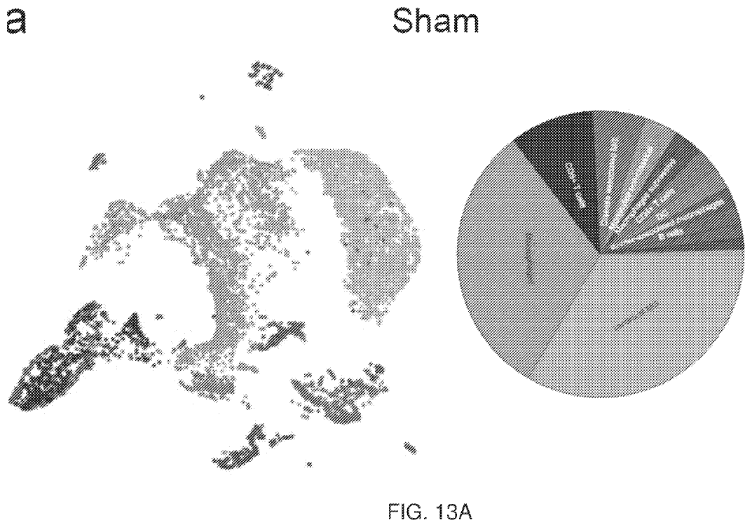

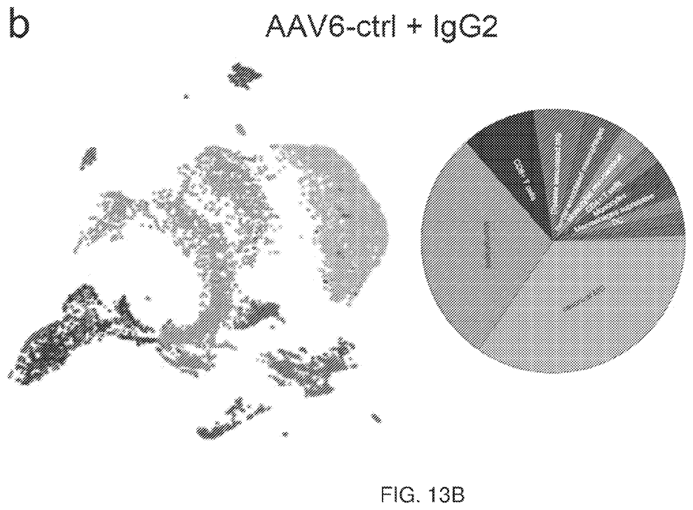

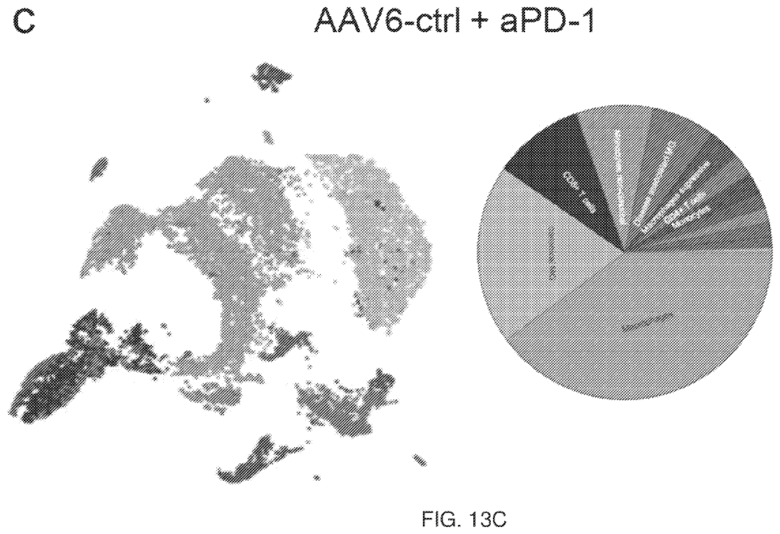

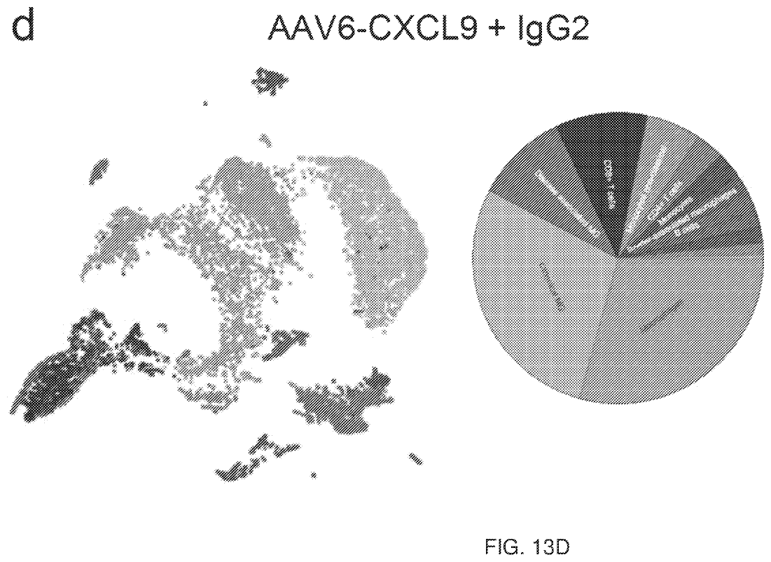

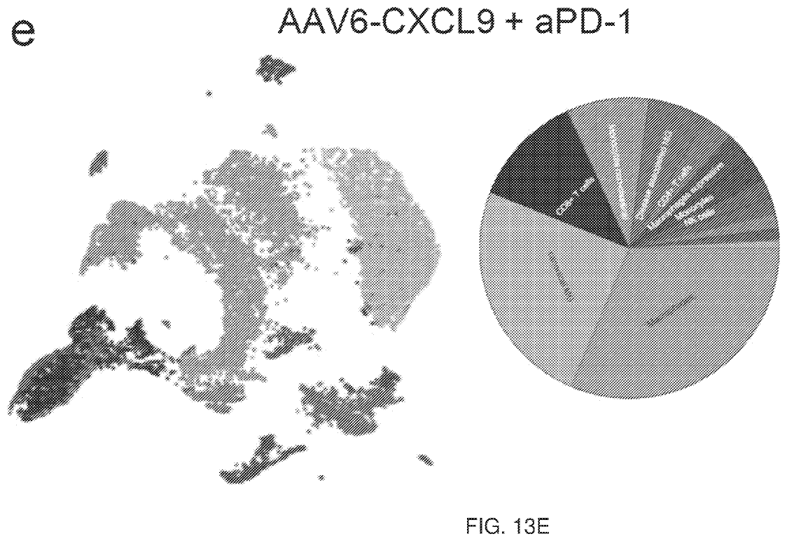

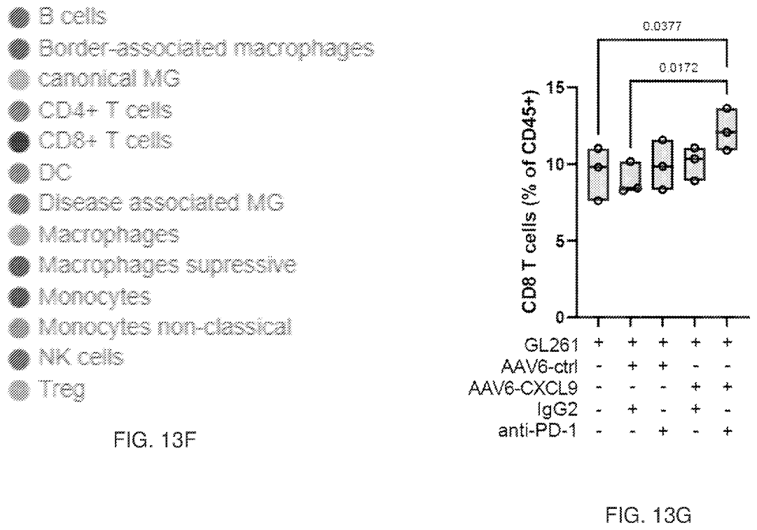

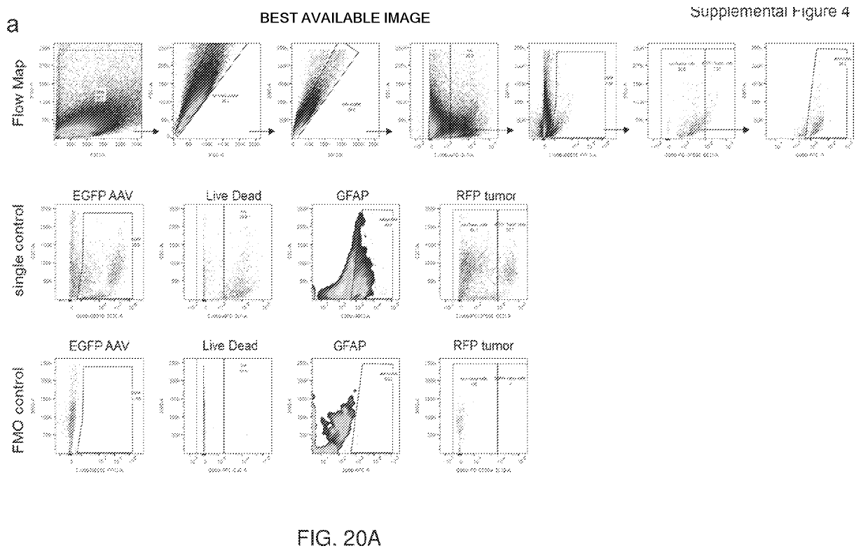

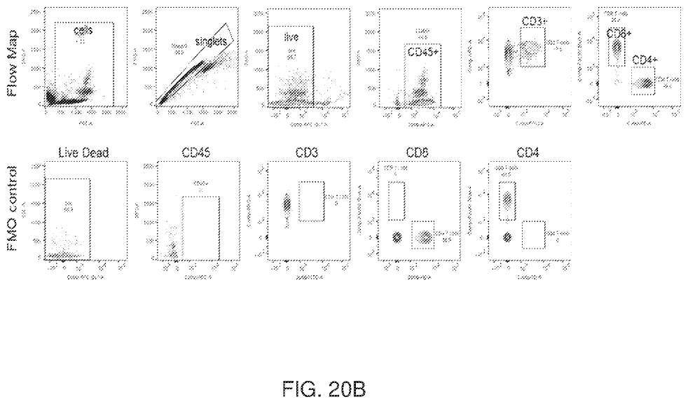

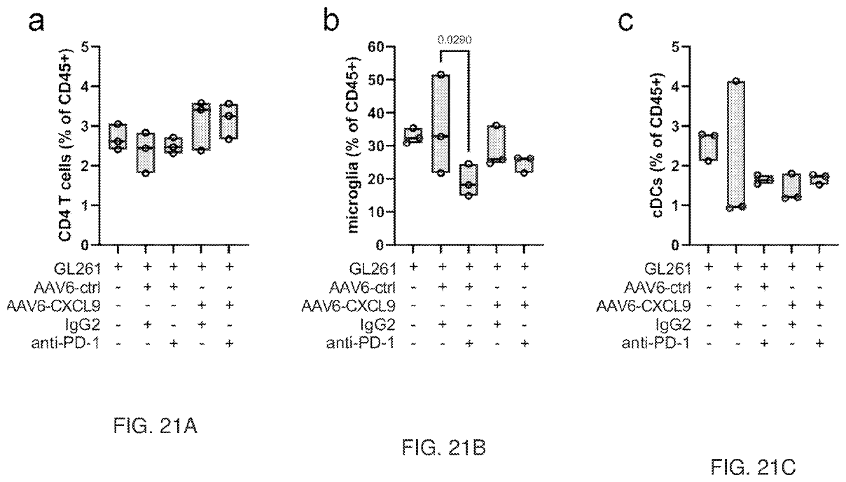

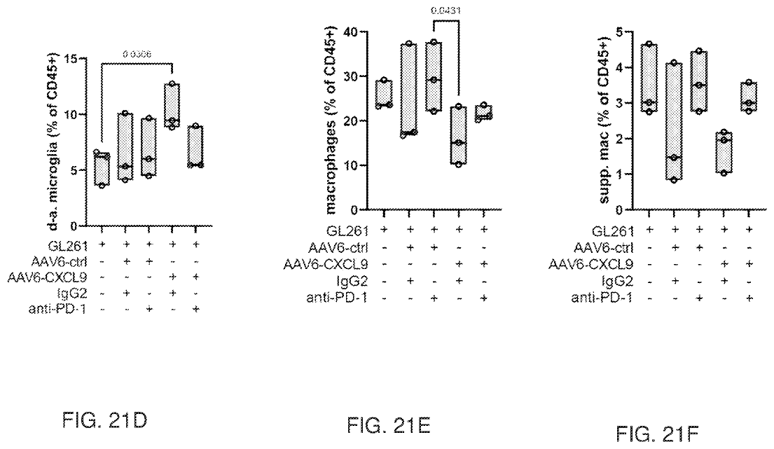

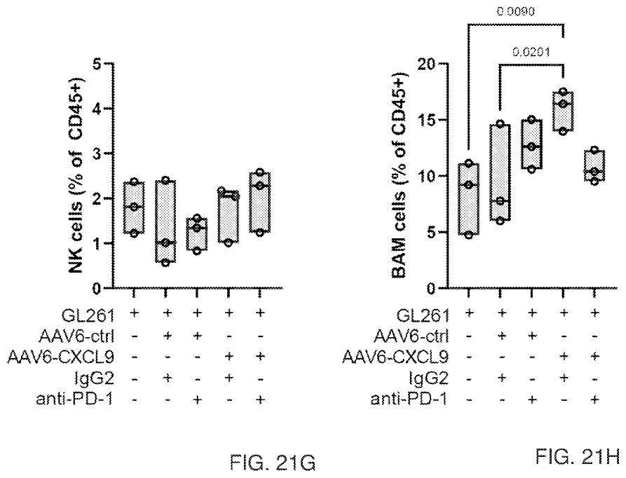

In an effort to define the immunological landscape of AAV6-CXCL9 treated tumors with or without concurrent anti-PD-1 ICB, single cell RNA sequencing (scRNAseq) was performed on CD45-positive cells isolated from GL261 tumors collected on day 15 of treatment as outlined in FIG. 4D. Dimensionality reduction using uniform manifold approximation and projection (UMAP) was performed on 52,344 cells collected across five treatment groups: sham (saline), AAV6-ctrl+IgG, AAV6-ctrl+aPD-1, AAV6-CXCL9+IgG, and combination AAV6-CXCL9+aPD-1, 3 mice per group (FIG. 13A-E). Top differentially expressed genes from each pooled population were identified, and cluster cell types were defined using the expression of known marker genes resulting in the identification of 13 unique cell clusters (FIG. 13F and Table 1). Analyses of lymphocyte tumor recruitment across treatment groups recapitulate the earlier observation, with combination therapy yielding a significant increase in total infiltrating CD8 T lymphocytes (FIG. 13G), identified using the gene expression markers Cd3d, CD8a, Cd8b1 as previously described. T regulatory lymphocytes (Treg), defined by Cd4, Foxp3, and I12ra gene expression, were also increased in response to combination therapy, although collectively these represent <1% of the total tumor-associated immune population (FIG. 13H). Increased tumor infiltration by monocytes, classified by high Ly6c1 expression, was observed across all treatment groups as compared to sham control mice (FIG. 13I), with an enrichment of non-classical monocytes characterized by Spn, Cx3cr1, and Tnfrsf1b expression in groups receiving anti-PD-1 treatment (FIG. 13J). Graphical summaries for all remaining cells clusters in response to each treatment are shown in FIGS. 21A-21H.

### Example 2

### Combination of rAAV6-CXCL9 and Anti-PD-1 Treatment Increases Cellular Cross-Talk in Lymphocytes

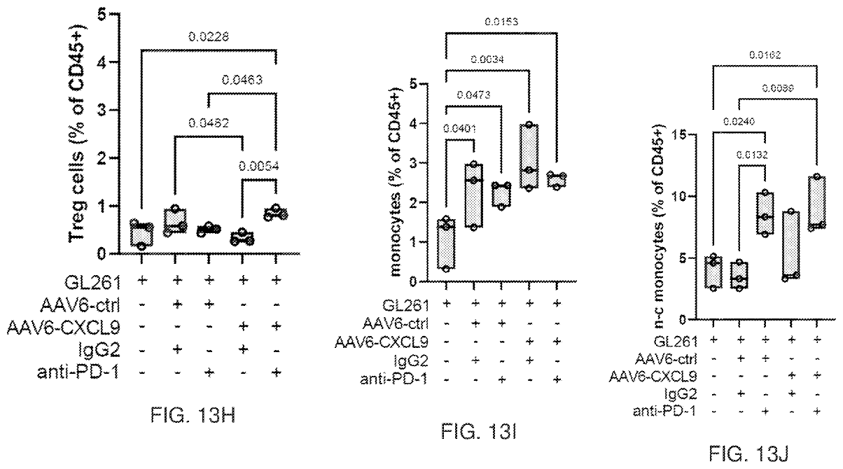

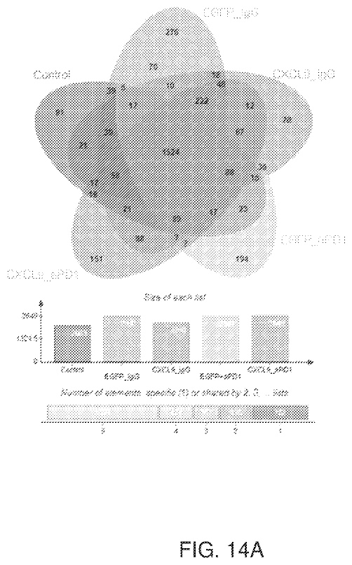

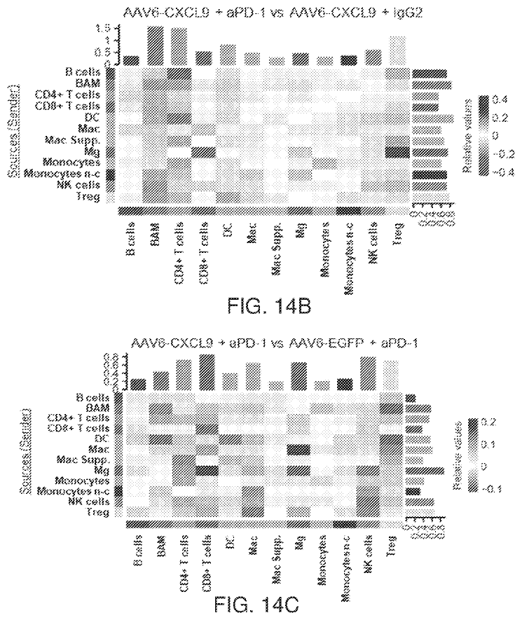

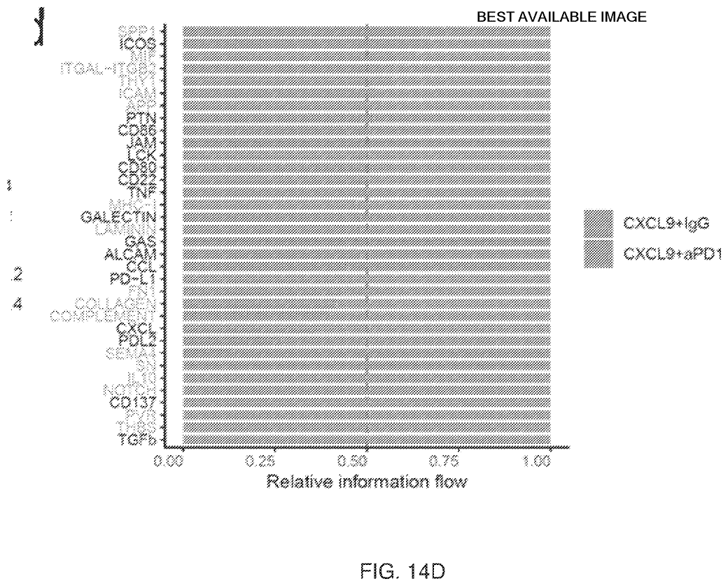

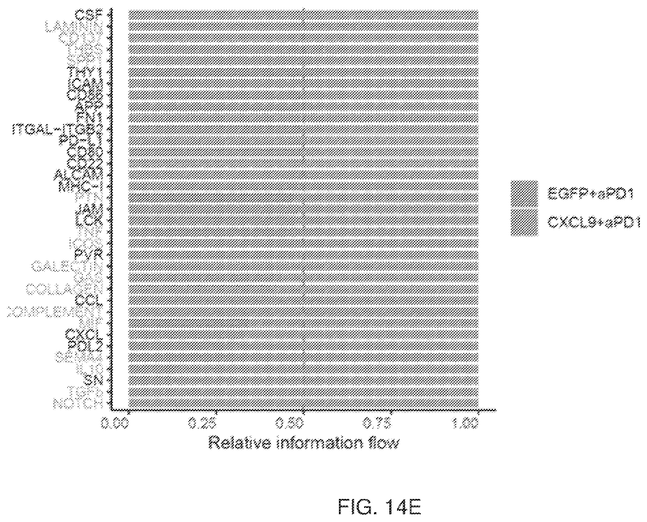

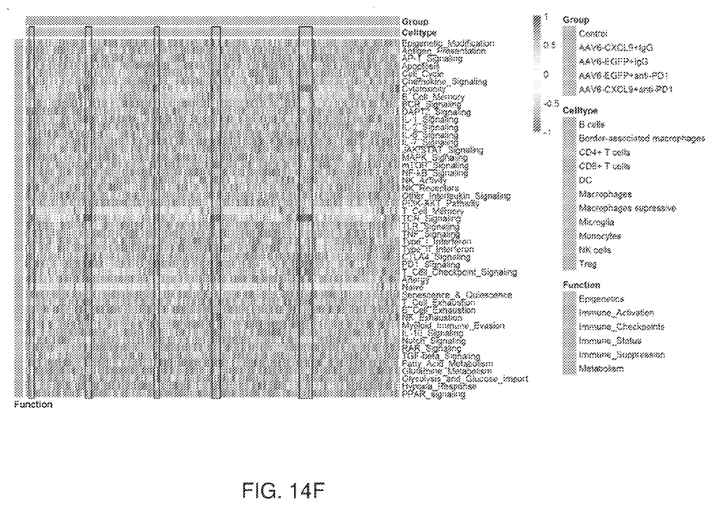

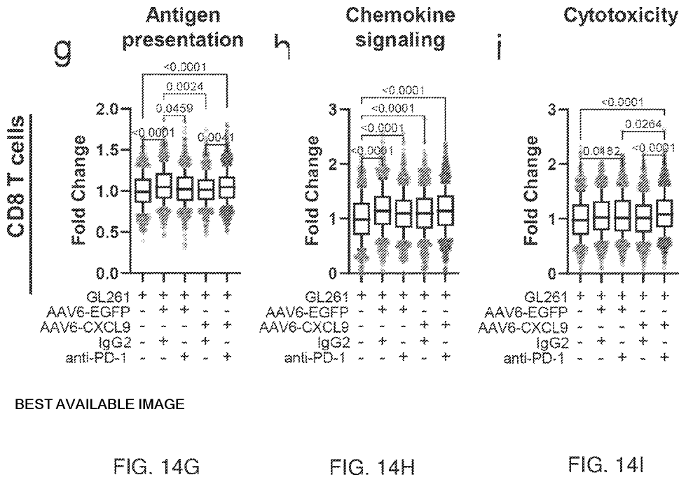

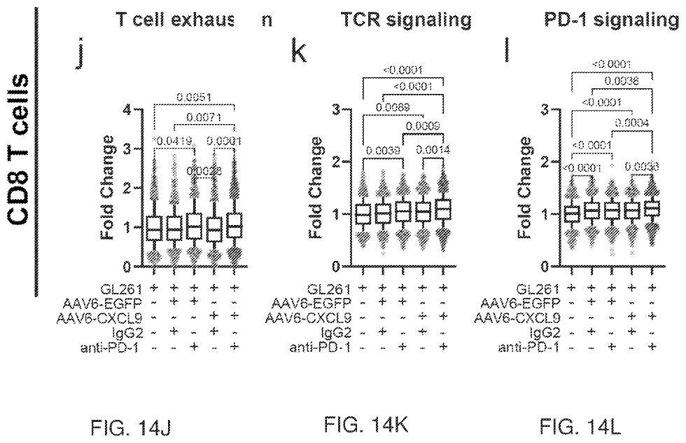

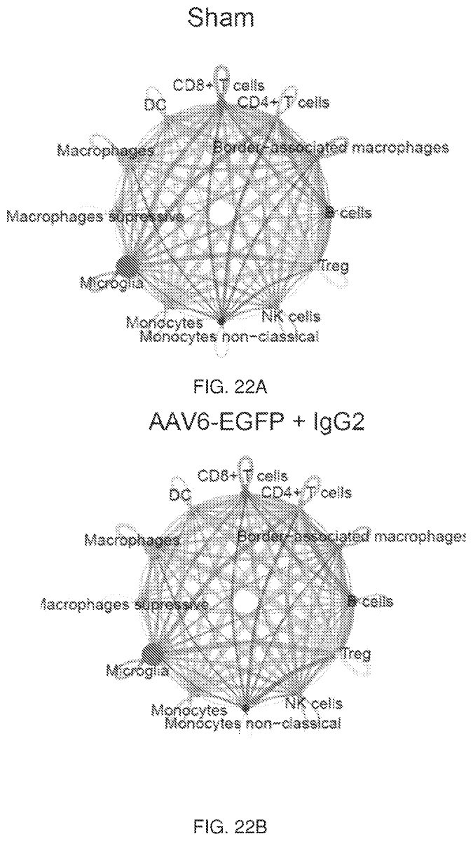

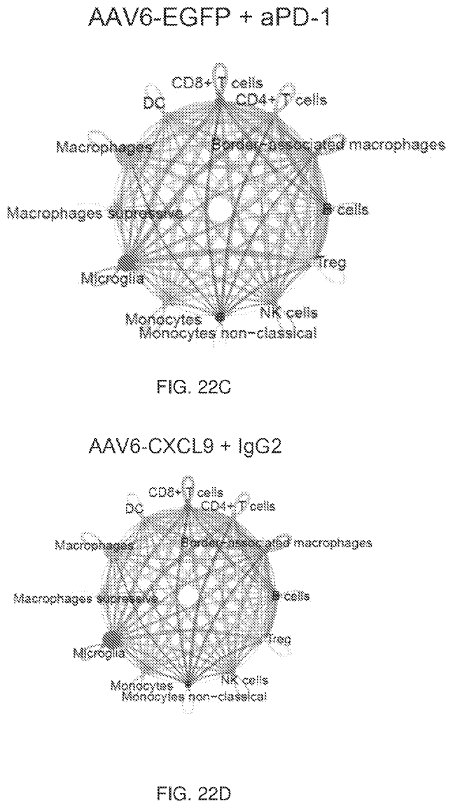

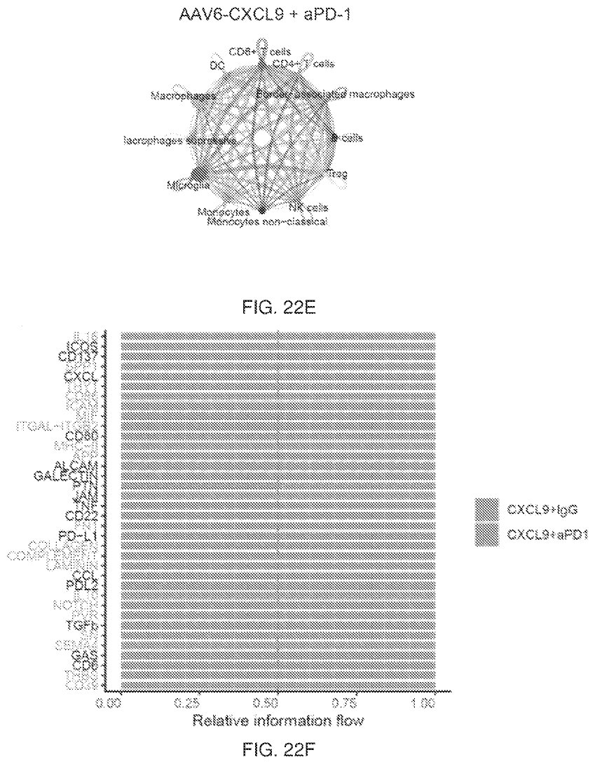

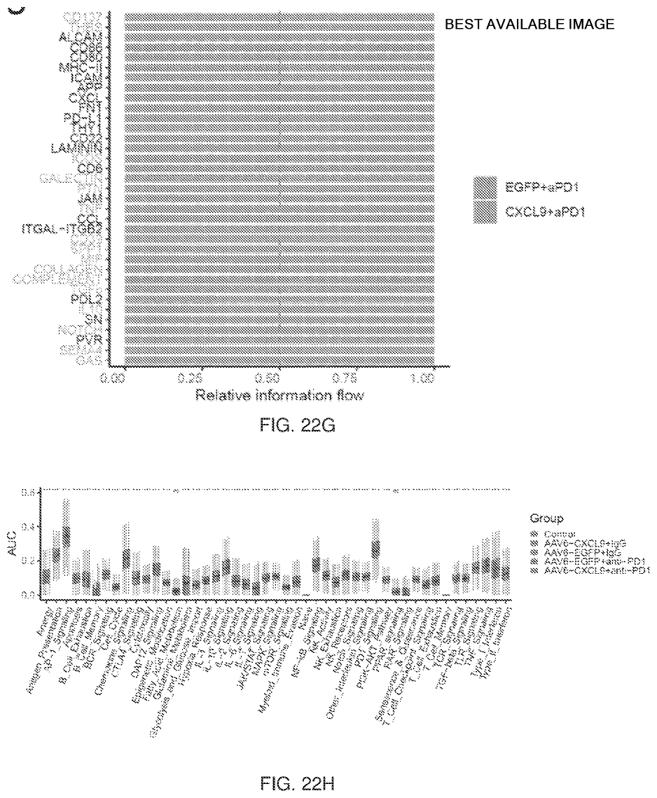

As shown in FIG. 14A, 2,260 differentially expressed genes (DEGs) associated with AAV6-CXCL9 treatment, 2,607 DEGs associated with anti-PD-1 treatment, and 2,649 DEGs associated with these treatments combined were identified. Of these, 70, 194, and 151 DEGs appear to be unique to each given treatment strategy, respectively, and may provide unique insight toward treatment impact on immune cell functional states. Through transcriptional expression of distinct ligands and receptors, cell-type-specific interactions was inferred, providing additional insight towards the inflammatory profile of tumors and how they change in response to treatment. Using predefined cell clusters, a simplified DEG set was established for each. DEGs were then queried against public ligand-receptor databases (see Methods). Summary results are shown in Chord Plots, where line thickness represents the number of predicted interactions between two defined cell clusters (FIG. 22A-22E). Next, direct comparisons of interactome activity were performed between treatment groups to elucidate heightened or decreased connectivity associated with AAV6-CXCL9 and anti-PD-1 ICB, where heatmap relative values in red indicate increases and blue decreases in prospective ligand-receptor interactions. In evaluating AAV6-CXCL9 in combination with either anti-PD-1 ICB or IgG2 control to resolve the contributions of ICB, notable increases in signals emanating from each macrophages (Mac), border-associated macrophages (BAM), microglia (Mg), and NK cells signaling to CD8+ and regulatory T cells were observed (FIG. 14B). Decreased incoming signals were noted in BAMs, CD4+ T cells, and dendritic cells (DCs) stemming from nearly all cell clusters (FIG. 14B). Cell-cell interactions associated with AAV6-CXCL9 shown in FIG. 14C reveal heightened communication directed toward both CD4+ and CD8+ T cell subsets, and NK cells prompted by all clusters excluding B cells and DCs. Signaling originating from all lymphocyte populations, and most innate immune cells including Macs, Mgs, Monocytes, and NK cells was increased, suggesting that AAV6-CXCL9 treatment broadly stimulates immune activity.

Given that combination treatment promotes CD8 T cell tumor infiltration which is required for anti-tumor efficacy, it may be beneficial to resolve how treatment might impact CD8 T cell effector function via pathway analysis of DEGs specifically within these cells. Comparative pathway analysis between CD8 T cell DEGs shows selective enrichment of thrombospondin (THBS), poliovirus receptor (PVR, CD155), CD137 (4-1BB), fibronectin-1 (FN1), laminin, and major histocompatibility complex class I (MHC I), among others, as uniquely affiliated with combination therapy when compared to AAV6-CXCL9 plus IgG2 control (FIG. 14D). These data suggest that anti-PD-1 treatment prompts T cell activation via CD137, but also reciprocal immune suppression via CD155 given its inhibitory function as a ligand for T cell immunoreceptor with immunoglobulin and ITIM domain (TIGIT). FN1, laminin, and THBS are major constituents of the extracellular matrix, and when produced by lymphocytes have been described to support cell-cell engagement, transendothelial migration, and lymphoproliferation. DEG comparisons between combination therapy and AAV6-EGFP control plus anti-PD-1 ICB treatment reveals selective pathway enrichment of macrophage migration inhibitory factor (MIF), growth arrest specific (GAS), galectin, inducible T cell co-stimulator (ICOS), tumor necrosis factor (TNF), and pleiotrophin (PTN) as a result of AAV6-CXCL9 treatment (FIG. 14E). These data infer that AAV6-CXCL9 directly promotes CD8 T cell activation through increased ICOS and TNF expression, T cell migration via PTN, and reciprocally enhances innate immune stimulation of NK cells and myeloid cells via GAS and MIF secretion, respectively. It also reveals galectin-9 as a possible mechanism for CD8 T cell acquired exhaustion. Both anti-PD-1 ICB and AAV6-CXCL9 treatments stimulate NOTCH, TGFβ, IL-10, SEMA4, CXCL, Complement, and CCL pathway activation (FIG. 14D-14E), each with varying impact on T cell maturation, effector function, homeostasis, survival, and migration. Comparative pathway analysis was also performed for CD4 T cells across treatment groups (FIGS. 22F-22G).

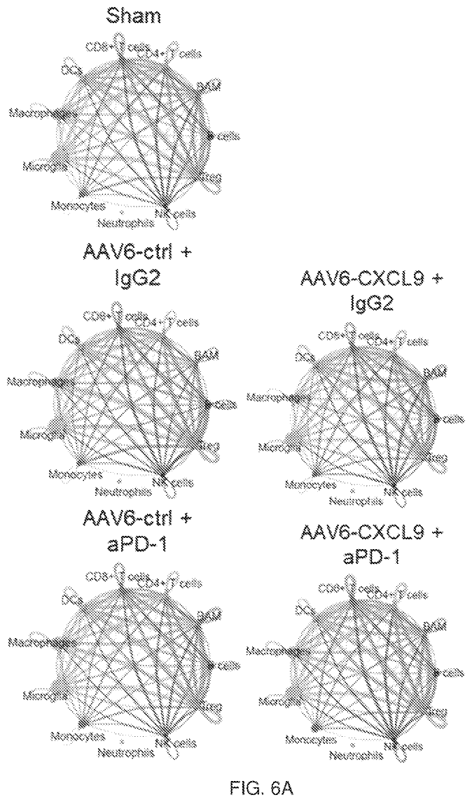

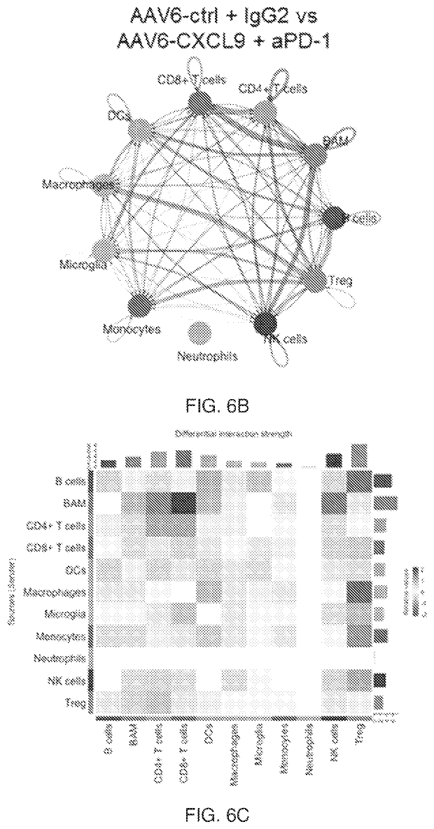

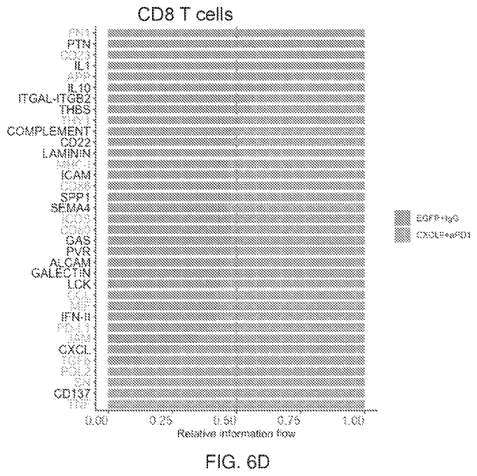

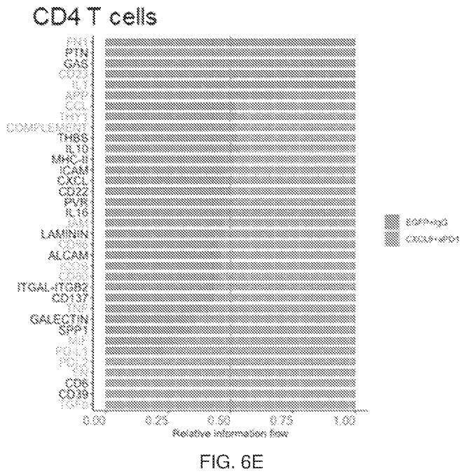

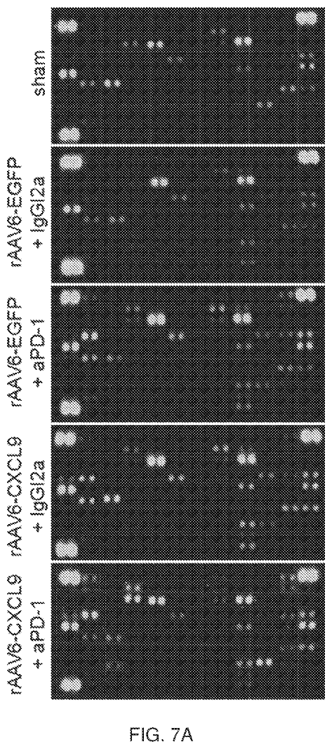

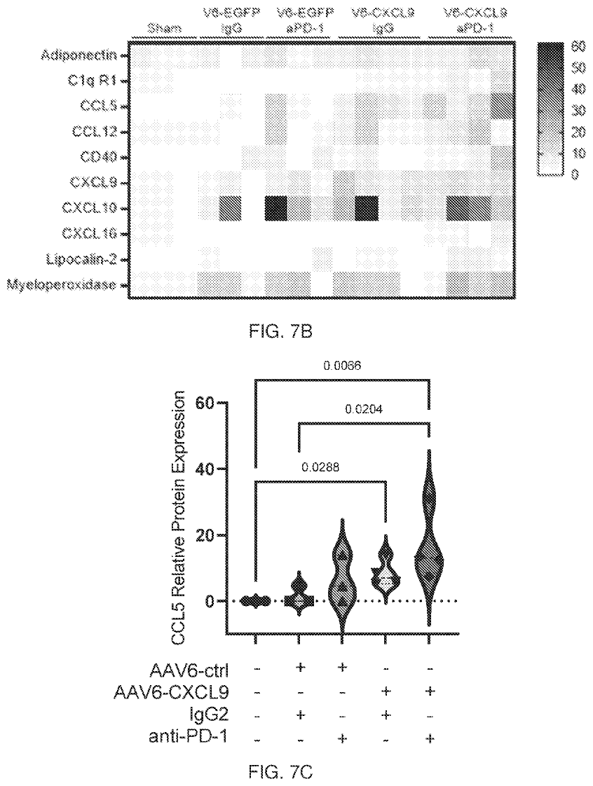

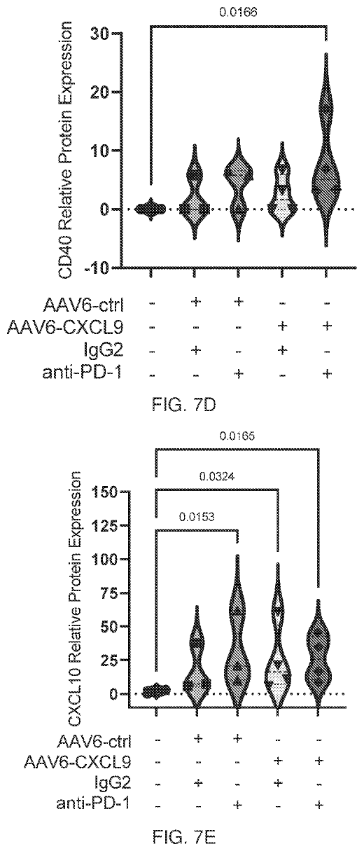

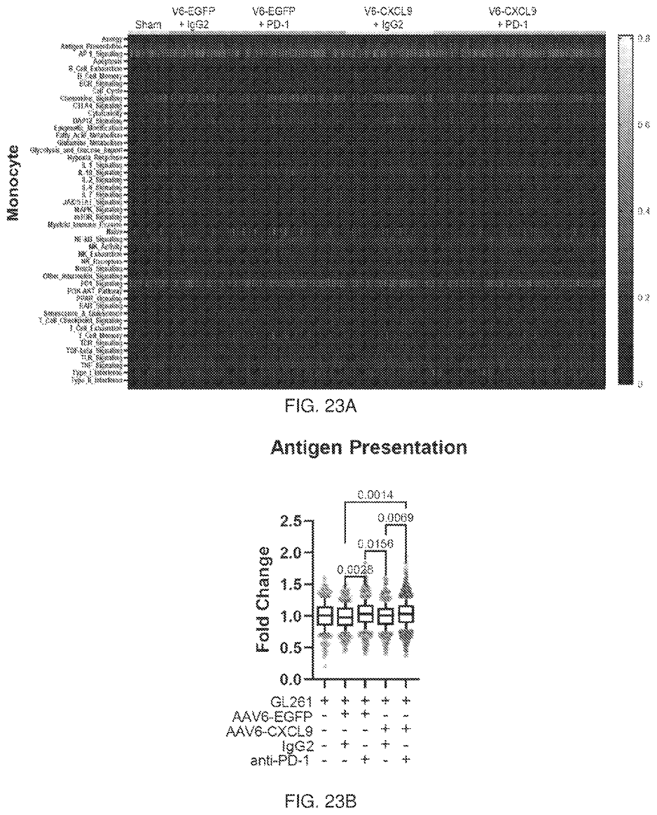

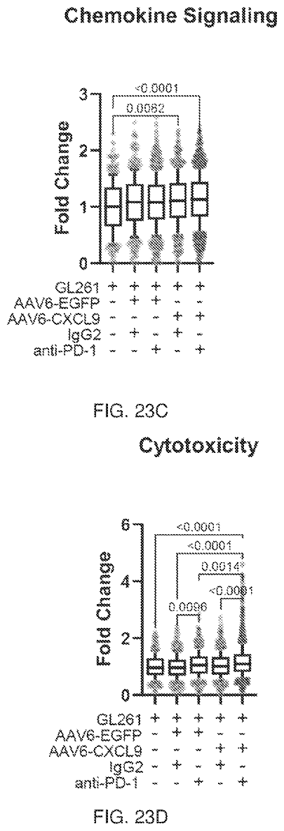

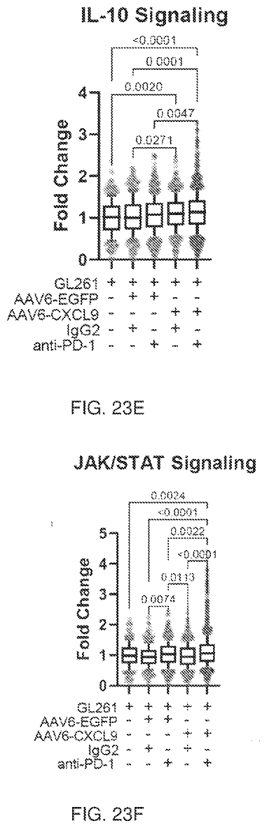

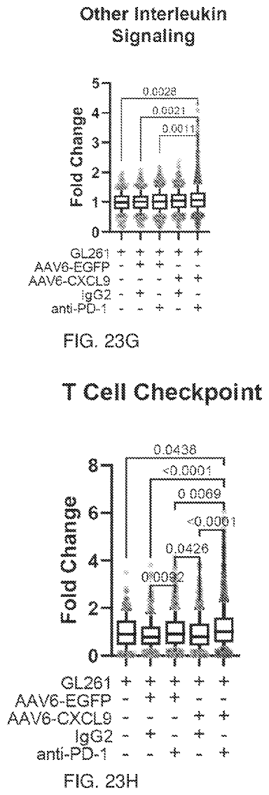

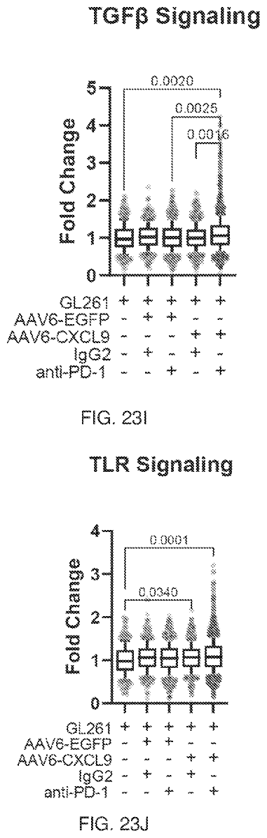

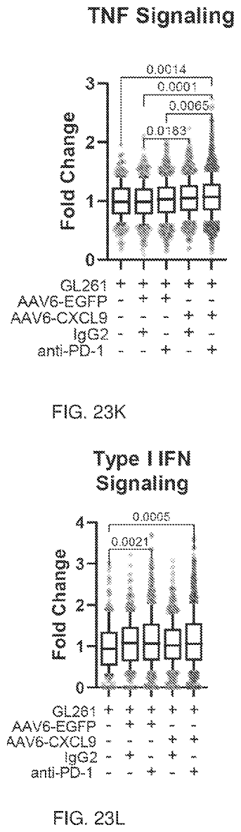

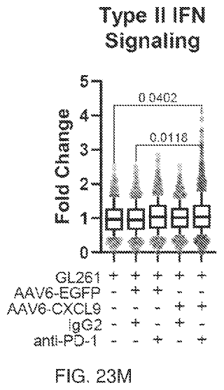

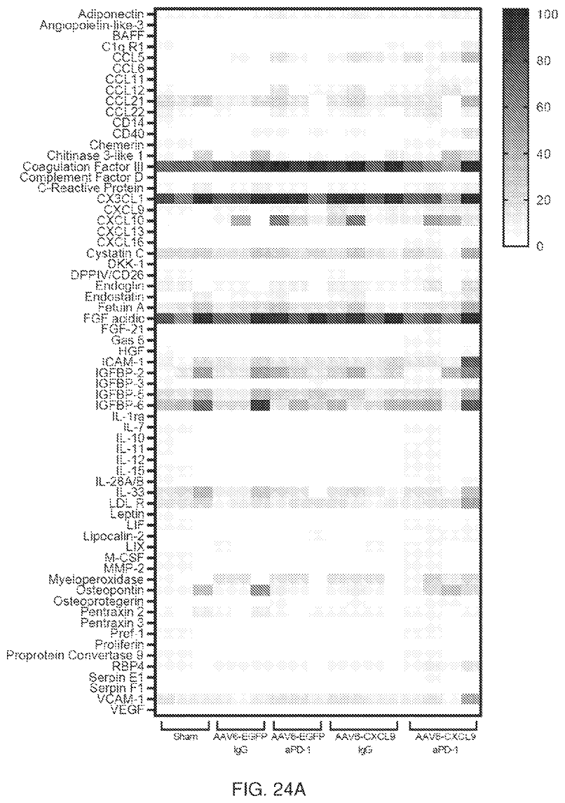

The nanoString nCounter® Immune Exhaustion Panel was used to further characterize immune status and inflammatory signatures associated with each respective treatment. A summary of pathway activation across all cell subsets in response to individual treatments is shown in FIGS. 6F, 22H, with CD8 T cell clusters outlined in black for each treatment group. Through deeper analysis of differential pathway activation, specifically in the CD8 T cell subset, heightened antigen presentation (FIG. 14G), chemokine signaling (FIG. 14H), cytotoxicity (FIG. 14I), T cell exhaustion (FIG. 14J), TCR signaling (FIG. 14K), and PD-1 signaling (FIG. 14L) were observed. In particular, combination AAV6-CXCL9 plus anti-PD-1 ICB was associated with the highest increase in cytotoxicity, TCR signaling, and PD-1 signaling. Increased T cell exhaustion appears to be associated with AAV6-CXCL9 treatment. Given that monocyte tumor infiltration was additionally increased in response to treatment (FIG. 13I-13J), pathway activation in these cells was evaluated to better understand their functional status. Enhanced activation was observed across 12 pathways, including antigen presentation, chemokine signaling, cytotoxicity, IL-10 signaling, JAK/STAT signaling, other interleukin signaling, T cell checkpoint, TGFβ signaling, TNF signaling, Type I interferon signaling, and Type II interferon signaling (FIG. 23A-23M). Of these, AAV6-CXCL9 treatment appears to be associated with increased antigen presentation, cytotoxicity, JAK/STAT signaling, and Type I interferon signaling, where anti-PD-1 ICB induces IL-10 signaling, TLR signaling, and TNF signaling. Together these data suggest that treatment may augment the pro-inflammatory function of these cells.

### Cytokine Profiling of Combination AAV6-CXCL9 Plus Anti-PD-1 Treatment

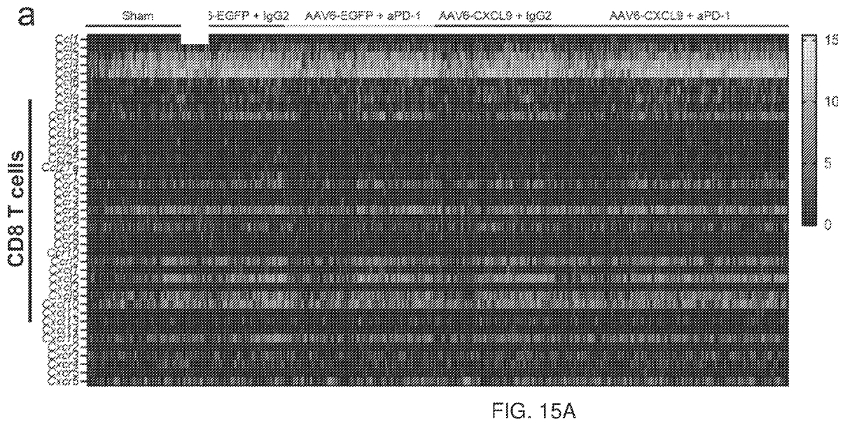

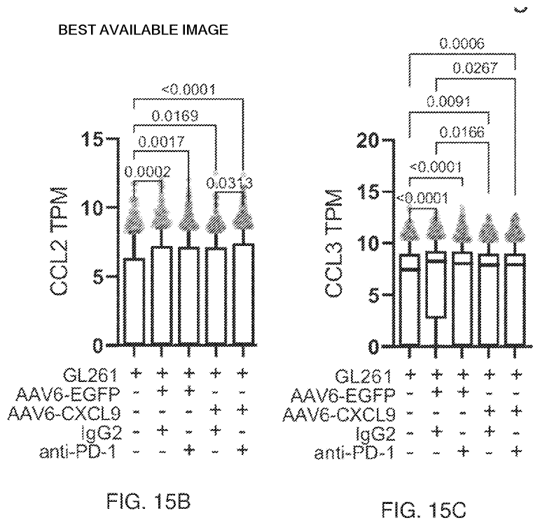

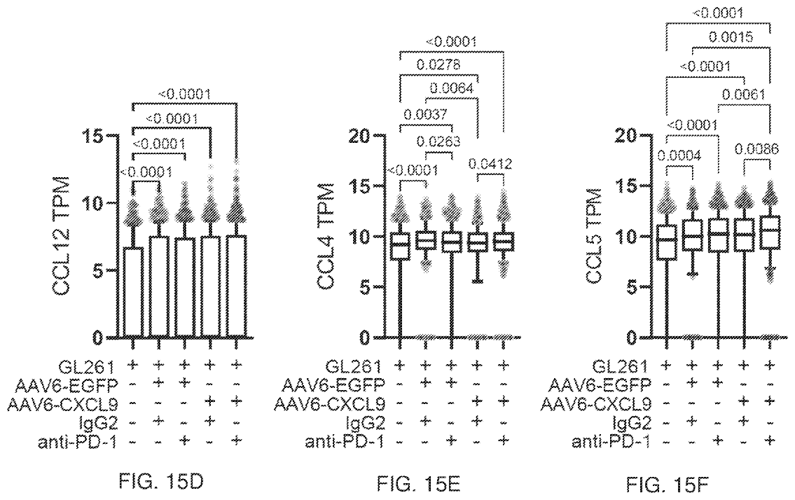

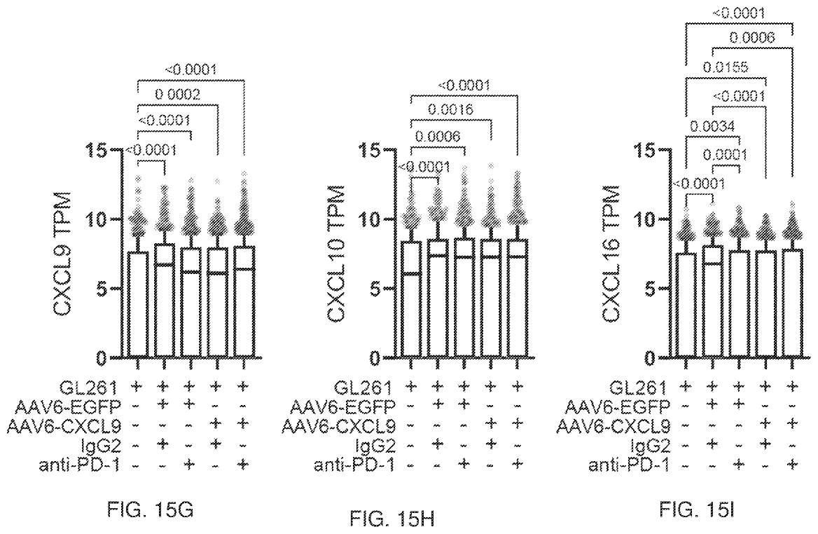

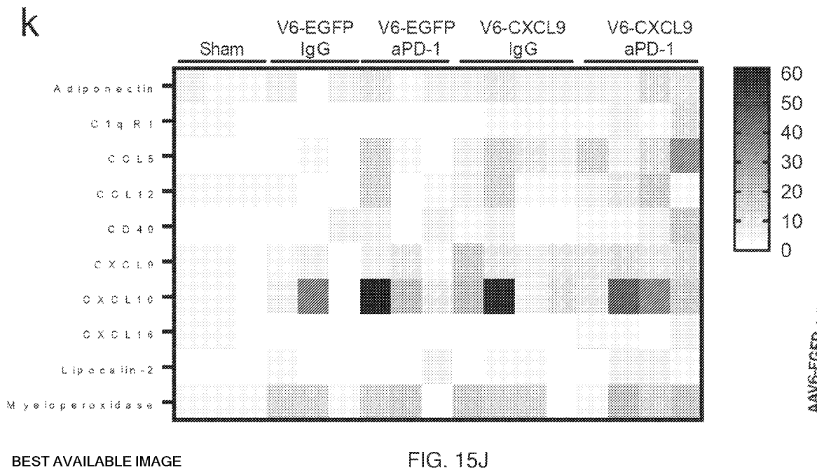

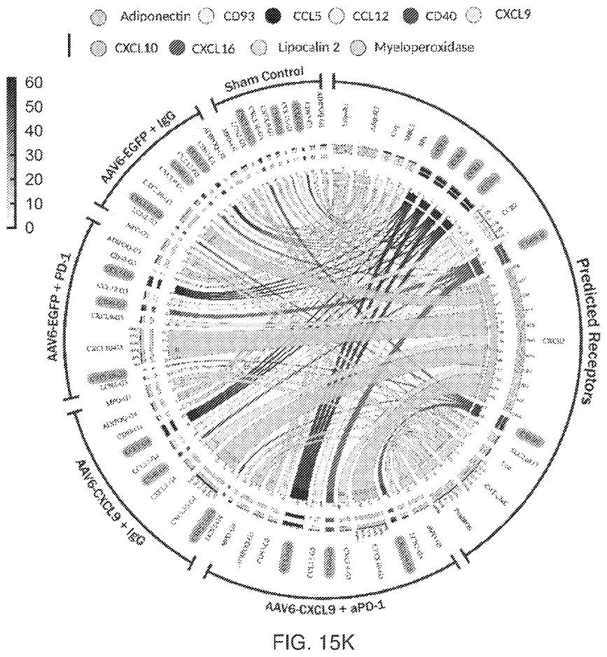

As combination therapy increases DEGs of both the CCL and CXC superfamily of secreted chemokines and cytokines, it may be beneficial to parse out transcriptional changes within CD8 T cells as an additional means to evaluate the activation state of these cells given the central role of these ligands in directing migration and activation of immune cells during inflammation. A summary of all CCL and CXC family ligand and receptor transcripts expressed by CD8 T cells is presented in the heatmap in FIG. 15A. CD8 T cell mediated stimulation of monocytes/macrophages is evidenced by increased transcription of CCL2, CCL3, and CCL12 across all treatment groups as compared to sham control (FIG. 15B-D). CCL4 transcription was also increased (FIG. 15E), indicative of NK stimulation by CD8 T cells. CCL5 was found to be the most differentially upregulated soluble ligand in combination treated CD8 T cells as compared to all other treatment groups (FIG. 15F), and is strongly indicative of CD8 T cell effector function. While AAV6 delivered CXCL9 transgene expression predominantly emanates from tumor-reactive astrocytes, scRNAseq data shows that each anti-PD-1 and AAV gene therapy induces CXCL9 transcription within CD8 T cells (FIG. 15G) additionally demonstrating immune activation as a result of treatment. CXCL10 was also found to be transcriptionally upregulated in response to anti-PD-1 and AAV gene therapy (FIG. 15H), which prompts further CD4, CD8, and NKT lymphocyte recruitment. Altogether, these data support that combination AAV6-CXCL9 and anti-PD-1 ICB both increases lymphocyte trafficking to intracranial GBM tumors and potently stimulates effector lymphocyte cellular communication and activation.

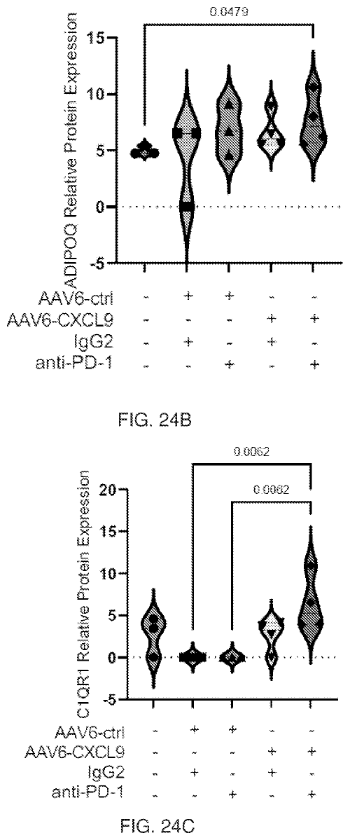

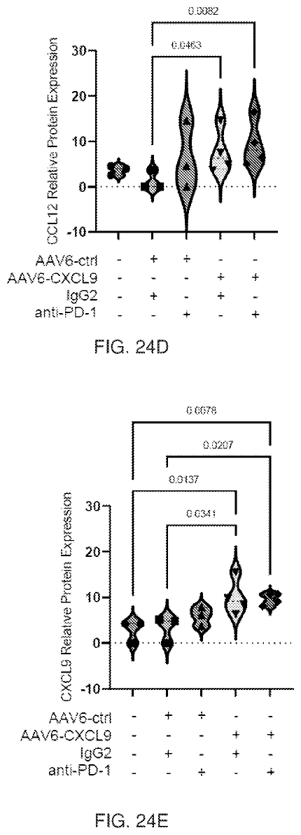

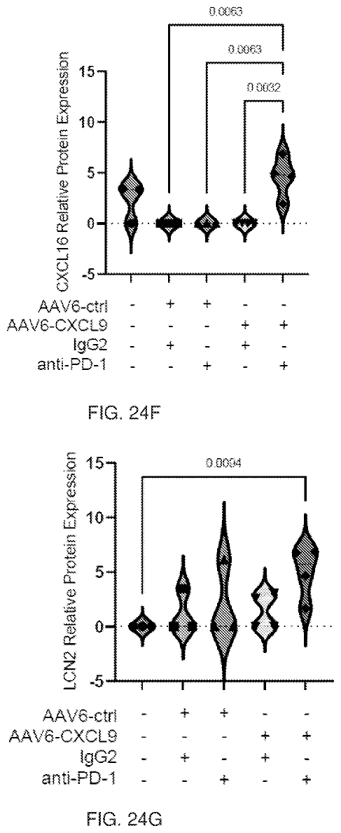

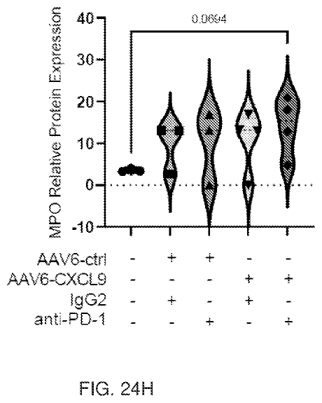

As described above, secreted cytokines can influence the trajectory of tumors in a multitude of ways-reprogramming tumor-associated cells and suppressing infiltrating inflammatory subsets which allows for tumor tolerance, progression, metastasis, and even therapeutic resistance or, alternatively, creating an environment favorable for innate and adaptive immune activation to facilitate tumor rejection. Moreover, the cytokine profile of a tumor may serve as predictive and/or therapeutic biomarkers allowing for the detection of tumor presence, forecasting therapeutic response, and can also be used to guide therapeutic choices. A large-scale cytokine proteomic assessment of single agent and combination treated tumors was performed to identify candidate biomarkers of response to therapy. Tumors were resected ten days after the onset of treatment as shown in FIG. 4D. Of the 111 soluble murine proteins on the array, relative expression of 65 were detected in treated and/or control GL261 tumor samples as summarized in FIG. 24H, with representative cytokine immunoblots shown in FIG. 15J. 10 secreted factors were identified as differentially expressed as compared to sham control tumors following either single or combination treatment with AAV6-CXCL9 and anti-PD-1 ICB: ADIPOQ, C1QR1 (CD93), CCL5, CCL12, CD40, CXCL9, CXCL10, CXCL16, LCN2, and MPO (FIG. 15J, FIG. 24B-24H). Of these, CCL5, CD40, and CXCL16 were most potently induced by combination treatment. These markers are highly indicative of lymphocyte presence and activation, where CCL5 is a potent pro-inflammatory ligand manufactured principally by CD8 T lymphocytes, and CD40 is a co-stimulatory ligand that triggers lymphocyte proliferation and cytokine production. Of note, elevated CCL5 ligand expression demonstrates concordance with scRNAseq data (FIG. 15F). In addition, CXCL9, CXCL10 and CXCL16 are strong chemotactic signals for lymphocyte recruitment. Both CXCL10 and CXCL16 are induced by interferon gamma (IFNγ) and tumor necrosis alpha (TNFα), powerful catalysts of innate and adaptive inflammation. A summary of treatment-induced secreted ligands and known receptor interactions are depicted via circular interactome analysis performed using Circos® visualization software, revealing insight towards immune reprogramming that occurs in response to each respective treatment (FIG. 151). These data combined validate that AAV6 delivery of CXCL9 to the tumor microenvironment in tandem with anti-PD-1 ICB not only facilitate lymphocyte recruitment to GBM tumors, but also reprograms the immunological landscape towards a pro-inflammatory phenotype.

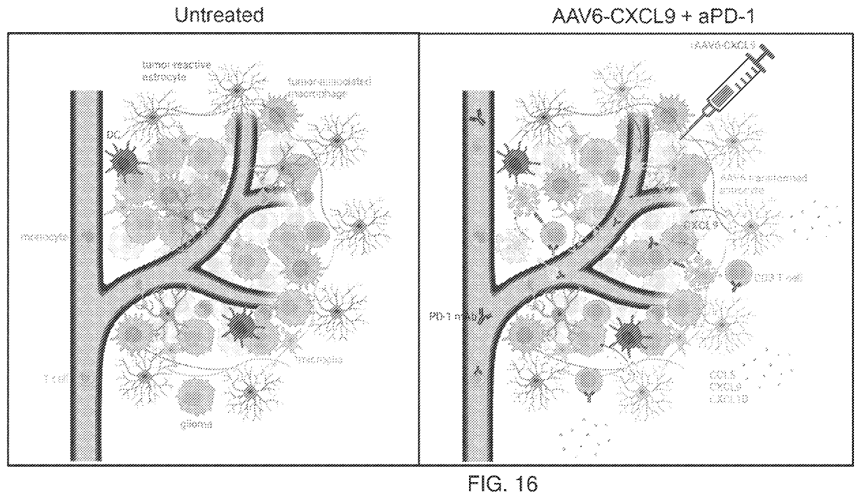

In summation (FIG. 16), intra-tumor delivery of AAV6 encoded CXCL9 results in the production of a pro-lymphocyte chemotactic gradient by transduced tumor-reactive astrocytes. This, in concert with anti-PD-1 ICB, significantly increases tumor infiltration by lymphocytes likely through CXCL9 engagement with its cognate receptor expressed by these cells-CXC motif chemokine receptor 3 (CXCR3). In particular, CD8 T lymphocytes are the premier arbiters of anti-tumor response, where depletion of this lymphocyte subset negates therapeutic efficacy. Moreover, CD8 T cell effector activation and function is evidenced by heightened expression of co-stimulatory molecules, such as 4-1BB and ICOS, and production of pro-inflammatory chemokines and cytokines. Beyond CD8 T lymphocyte activation, AAV6-CXCL9 and anti-PD-1 ICB appear to contribute widespread immunological activation, demonstrated by heightened cellular cross-talk across numerous immune clusters, and protein detection of pro-inflammatory molecules. Notably, CCL5, CXCL9, CXCL10, and CD40 are detected in response to combination therapy within resected tumors, and may serve as biomarkers of therapeutic response.

### AAV6-CXCL9 Sensitizes Preclinical GBM to Anti-PD-1 Checkpoint Blockade

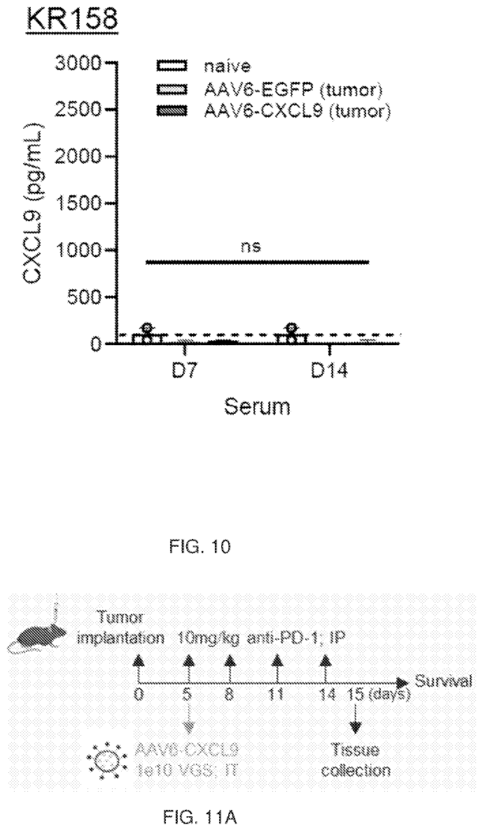

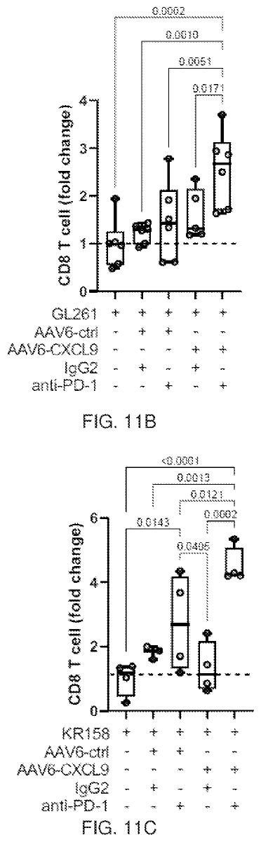

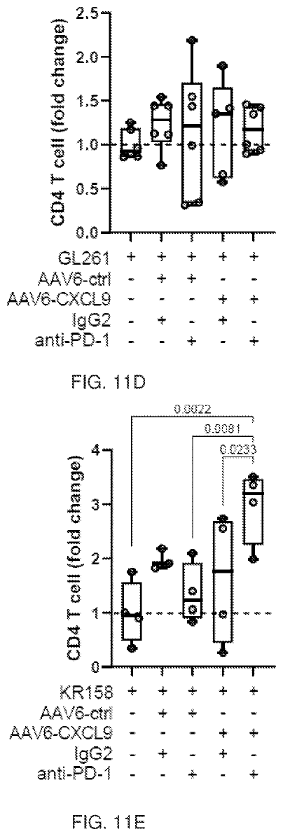

To assess if enhanced lymphocyte recruitment and immunological reprogramming through combination treatment could produce anti-tumor responses against GBM, survival analyses were performed in both the GL261 and KR158 syngeneic model systems. 5 days following tumor implantation, AAV6 encoding CXCL9 or EGFP control transgene was injected intratumorally, with anti-PD-1 ICB (10 mg/kg) administered intraperitoneally for a total of 4 doses given every 72 hours (FIG. 11A). In the GL261 model, anti-PD-1 ICB was observed to produce a small, but non-significant increase in overall survival as compared to sham treated control animals (p=0.060), where AAV6-CXCL9 treatment yielded no survival benefit as a monotherapy (FIG. 4A). Combination treatment significantly improved overall survival, with 50% of animals exhibiting durable outcomes (FIG. 4A). Similar results were observed in the KR158 model, which carries a low mutational burden and is recalcitrant to immunotherapy, with combination treatment significantly improving median survival, and long-term survival observed in 25% of this cohort (FIG. 4B). As an additional metric to validate the ability of combination AAV6-CXCL9 plus anti-PD-1 ICB to immunologically transform GBM tumors, GL261 tumors were implanted in GREAT transgenic mice to evaluate tumor-wide IFNγ expression following treatment as described in FIG. 3D. IFNγ was detected in combination treated tumors evidenced by EYFP signal detection via 3D IHC (FIG. 12A). Immunolabeling of tissues for CD45 confirms that EYFP (IFNγ)+ cells are immune cells (FIG. 12B), indicative of pro-inflammatory immune activation.

To determine if CD8 lymphocytes contribute to the therapeutic survival effect, combinatorial treatment was repeated with concomitant CD8 depletion (FIG. 4E) in the GL261 model. On study day 18, it was observed that all animals treated with CD8 depleting antibodies had no detectable levels of circulating CD8 T lymphocytes, and no changes in the quantity of circulating CD4 T lymphocytes (FIG. 4F, FIG. 24B). CD8 depletion reversed the survival benefit observed with combination AAV6-CXCL9 plus anti-PD-1 ICB, and this cohort progressed as quickly as control treated subjects (FIG. 4G). To determine if combination treatment could confer long-term immune memory formation, a GL261 tumor rechallenge was performed in long-term survivors (>55 days) that had received AAV6-CXCL9 plus anti-PD-1 ICB. No observable residual tumor was present from the initial tumor implantation during the second implantation. A second cohort of age-matched naïve animals was intracranially injected with GL261 as a control. Control animals all succumbed to tumor burden within 30 days of tumor implantation, whereas 100% of rechallenge animals remained disease free (FIG. 4H). These data confirm that therapeutic response to combination therapy is dependent on tumor infiltration by CD8 T lymphocytes as part of the adaptive immune cascade, and combination therapy can convey long-term immune memory protection against recurrence.

### Cell Culture

KR158B-luc (Kluc) glioma line (provided by Dr. Karlyne M. Reilly, NCI Rare Tumor Initiative, NIH) and GL261 cells have been verified histologically as high-grade glioma, and gene expression analysis confirmed appropriate haplotype background and expression of astrocytoma-associated genes. CT-2A were purchased from Millipore Sigma. Primary human glioma cells including L0, L1, L2, CA1, CA2, CA4, CA6, CA7, L23, L26, L31, L34, L38, L47, and HA2 were a kind gift from Dr. Brent A. Reynolds20. C8-D1A primary astrocytes were purchased from ATCC. All cells were cultured in DMEM (Fisher-Scientific) supplemented with 10% FBS (VWR) and 1% Penn-Strep (Life Technologies), and maintained at 370C in humidified conditions with 5% CO2. At the beginning of the study, cells were expanded, stocks made, and thawed vials were maintained in culture for no more than 3 weeks.

### In Vivo Studies

C57BL/6J (Strain #000664), CCR2RFPCX3CR1GFP (Strain #032127), and GREAT (Strain #017581) mice were purchased from Jackson Laboratory. On day 0, 5×104 tumor cells suspended in 50% methylcellulose and 50% saline (Fisher-Scientific) were stereotaxically injected into murine brain at a depth of 3 mm, 2 mm lateral to bregma, at a volume of 2 μl in 8-16-week-old animals. On day 5, AAV6 vectors were intratumorally injected in the same coordinates as tumor implantation. Where indicated, monoclonal antibody treatment (PD-1 ICB, IgG control, CD8a depletion) was administered beginning Day 5 via intraperitoneal injection and given every 72 hours. Protocols were reviewed and approved by the University of Florida Institutional Animal Care and Use Committee.

### Clinical Specimens

De-identified patient tissues were procured by the Florida Center for Brain Tumor Research (FCBTR) under the University of Florida Institutional Review Board protocols 201300482.

### Drug

In VivoMAb anti-mouse PD-1 and In VivoMAb rat IgG2a isotype control monoclonal antibodies were purchased from BioXcell, diluted in Sterile Saline 0.9% solution (Patterson Veterinary Supply, Inc.), and administered via intraperitoneal injection at a dose of 10 mg/kg given every 72 hours for a total of 4 doses. In VivoMAb anti-mouse CD8a and In VivoMAb rat IgG2b isotype control monoclonal antibodies were purchased from BioXcell, diluted in Sterile Saline 0.9% solution (Patterson Veterinary Supply, Inc.), and administered via intraperitoneal injection at a dose of 300 μg/mouse given every 72 hours for a total of 6 doses.

### AAV Protocol

HEK 293T cells (ATCC cat #CRL3216) were cultured to ~70% confluency in two Cellstacks (Corning cat #3269) per construct and transfected using PEI 25k MW (Polysciences cat #23966-1) for 3 days. The cells were then harvested via shaking and centrifugation until a cell pellet was formed. The pellet was then digested with a final concentration of 50U/mL of Benzonase (Sigma cat #E8263) and 0.5% sodium deoxycholate in a lysis buffer (150 mM NaCl, 50 mM Tris-HCl pH 8.4) for 30 minutes at 370 C. Following incubation, the supernatant was supplemented with 5M NaCl until a 1M final concentration was achieved. Afterwards, the supernatant was lysed via 3 freeze thaw cycles of −800C and 500 C. The lysate was spun down and supernatant transferred to an ultracentrifuge tube (Beckman cat #342414), where it is layered with discontinuous layers of iodixanol (Accurate Chemical cat #AN1114542) to separate out viral particles from the supernatant. This was spun for 1 hour at 180C at 69,000 rpm. The viral particles were isolated and removed, then washed four times in a dialysis column (Millipore cat #UFC910024) with PBS before being finally purified in a sterile filtration column (Millipore cat #UFC30DV00).

### AAV Quantification

The viruses were titrated by quantitative PCR (Bio-Rad CFX384) using custom probes designed to target the ITR sequences. First, 1 μL of the virus was treated with DNAseI (Thermo Fisher cat #18068015) for 15 minutes at room temperature, inactivated by heat and EDTA, protein coat of virus digested with Proteinase K (Thermo Fisher cat #25530049) and finished with a second heat-inactivation step. Following incubations, the sample was diluted and mixed with a Taqman PCR Master Mix (Thermo Fisher cat #4352042) and the custom designed probes (Thermo Fisher cat #4332078). The probe sequences were as follows: Forward-GGAACCCCTAGTGATGGAGTT (SEQ ID NO: 1), Reverse-CGGCCTCAGTGAGCGA (SEQ ID NO: 2), Probe-CACTCCCTCTCTGCGCGCTCG (SEQ ID NO: 6). The samples were then compared to a standard curve consisting of a linearized plasmid with ITRs from a range of 1e4 to 1e8 genomic copies per mL. The samples were then run on a standard program of 10 minute denature at 950C, then cycled 39 times at 950C at 1 minute and 600C at 30 seconds.

### Lentiviral Transduction of Tumor Cells

RFP labeled GL261 and KR158 tumor cells were transduced with a LentiBrite RFP Control Lentiviral Biosensor (Millipore-Sigma), MOI 50. Following cell expansion, RFP-positive cells were FACS sorted using a BD FACSAria-II cell sorter, yielding RFP-stable tumor cells.

### Proteome Arrays

Following resection, the right hemisphere (cerebellum removed) of murine brain (tumor-containing) were transferred to 1.5 mL microtubes, snap frozen in LN2, and stored at −80° C. until lysis. De-identified flash frozen patient GBM tissue was procured from the FCBTR. Tissue shavings were collected on dry ice and transferred to 1.5 mL microtubes. 300-500 μl PBS containing 1× Halt™ Protease/Phosphatase inhibitor (Thermo Fisher) and 1% Triton X-100 (Sigma) was added to samples and transferred to wet ice. Tissue was lysed manually using a 20-gauge needle attached to a 1 ml syringe followed by vortexing every 5 minutes for 30-60 minutes. Supernatant was collected following centrifugation at 10,000G at 4° C., and assayed for protein concentration using a NanoDrop Spectrophotometer. 0.75 mg of each human sample was used for the Human Chemokine Array Profiler (R&D Systems, ARY017), and 1 mg of each murine sample was used for the Mouse XL Cytokine Array (R&D Systems, ARY028) following manufacturer's instruction. Images were captured using BioRad ChemiDoc MP Imaging System with ImageLab 6.1 software over a series of exposure times. Mean voxel intensity per capture antibody was calculated using Imaris ×64 v9.7.0, and protein signal was normalized against internal reference controls. Detected protein and predicted receptor interactions were analyzed and visualized using Circos® 60.

### ELISA

Tissue specimens were collected at 1 and 2 weeks post-AAV6 injection. Peripheral blood was taken from the anterior vena cava, centrifuged at 12,000 rpm×10 minutes at room temperature (RT), and serum collected and stored at −80° C. Whole brain was resected, cerebellum removed, and divided into the tumor-bearing (AAV6 injected) and contralateral hemispheres. Naive brain and serum were collected and used to set the baseline for both week 1 and week 2 datasets. Tissue was snap frozen and stored at −800C until lysis. Tissue was lysed using RIPA buffer containing 2× Halt protease/phosphatase inhibitor cocktail (Thermo Fisher) with manual dissociation performed using a 20-gauge needle attached to a 1 mL syringe followed by vortexing every 5 minutes for 30-60 minutes, and maintained on ice. Following lysis, tissue samples were centrifuged at 12,000 rpm at 40C×10 minutes. Supernatant was collected and assayed for protein concentration using a NanoDrop. Protein concentrations were adjusted using RIPA buffer. MIG/CXCL9 ELISA (Thermo Fisher) performed according to manufacturer protocol. Serum diluted 1:2 using Assay Diluent B. Tissue sample concentration: 2 mg. All samples were run in duplicate.

### 3D Tissue Clearing and Immunolabeling

Brain tissue was collected after cardiac perfusion with cold saline followed by PBS supplemented with 4% acrylamide (Sigma-Aldrich), 0.05% N,N′-methylenebisacrylamide (Sigma-Aldrich), 4% paraformaldehyde and 0.25% VA-044 (TCI America). Tissues were stored at 40C for 3 days to allow hydrogel permeation of tissues. Following hydrogel polymerization at 370C ×3 hours, whole brain was sectioned to 2 mm and passively cleared over 3-7 days with PBS containing 200 mM boric acid (Sigma-Aldrich) and 4% sodium-dodecyl-sulfate (Fisher-Scientific), pH 8.5 at 500C. After clearing, samples were washed in PBS with 0.1% Triton X-100 for 2 days, and immunostained at 40C for 2 days with the following antibodies and stains: GFAP (Thermo Fisher, cat #PA1-10004), CD45 (Thermo Fisher, cat #14-0451-82), anti-chicken Alexa Fluor™ 647 antibody (Thermo Fisher, cat #A-21449), anti-rat Alexa Fluor™ 568 (Thermo Fisher, cat #A-11077), and either DAPI (Sigma-Aldrich) or DRAQ5 (Thermo Fisher) nuclear dye. Samples were whole-mounted onto slides using 62% 2,2′-Thiodiethanol (Sigma-Aldrich). Images were acquired using a Nikon A1RMP confocal microscope and analyzed using Imaris ×64 v9.7.0 software.

### Tissue Dissociation & Flow Cytometry

Brain tissue was digested using the Multi-tissue Dissociation kit (Miltenyi Biotec) on a gentleMACS™ Octo Dissociator with heat, followed by sample clean-up using Debris Removal Solution (Miltenyi Biotec) according to manufacturer's protocol. Tumor-infiltrating leukocytes were isolated using CD45 microbeads (Miltenyi Biotec) filtered through LS columns (Miltenyi Biotec) on a QuadroMACS Separator (Miltenyi Biotec) according to manufacturer's protocol. Blood samples were collected from the anterior vena cava, and RBC lysis performed using Pharm Lyse solution (BD Biosciences) per manufacturer's protocol. Samples were washed 2× with cold PBS. Unstained cells were reserved for unlabeled and FC controls, and dead cells were labeled with Zombie NIR™ Fixable Viability Kit (Biolegend) according to manufacturer's protocol. Cells were washed 2× in PBS containing 0.5% BSA (Sigma) and 2 mM EDTA (Thermo Fisher) FC buffer and blocked for 10 minutes on ice using TruStain FcX (Biolegend) prior to cell surface antigen labeling with the following antibodies: CD45-APC (Biolegend, cat #103112), CD3-FITC (Biolegend, cat #100204), CD4-PE (Biolegend, cat #100408), CD8-BV421 (Biolegend, cat #100738) for 45 minutes on ice. For astrocyte detection, cells were fixed and permeabilized using True-Nuclear™ Transcription Factor Buffer Set (Biolegend) following manufacturer's protocol following debris removal step, with no CD45 microbead isolation. Samples were labeled with Zombie NIR™ Fixable Viability Kit (Biolegend), blocked with TruStain FcX (Biolegend), and immunolabeled with GFAP-APC (ThermoFisher, cat #51-9792-82). Following immunolabeling, all samples were washed 2× with FC buffer and analyzed using a BD FACSymphony A3 flow cytometer.

### In Vitro Chemotaxis

GL261 or C8-D1A cells were plated in 24-well dishes at 1×105/well in pre-warmed complete media. AAV6-EGFP or AAV6-CXCL9 (RFP+) was added at a final concentration of 105 VGS. 24 hours following transduction, cells were transferred into the outer chambers of μ-Dishes with 3-well culture inserts (Ibidi), 104, suspended in 15 μl of growth-factor reduced Matrigel® (Corning). 40 μl of complete media was added following polymerization for 10 mins at 370C in humidified conditions with 5% CO2, CD3+ T cells were isolated from naïve C57BL/6 mouse spleen (8-12 weeks) using MojoSort CD3 T cell isolation kit (Biolegend) per manufacturer's protocol. T cells were labeled with Cell Trace Violet dye (CTV) (Thermo Fisher) per manufacturer protocol. 1×104 labeled T cells were suspended in 15 μl cold growth-factor reduced Matrigel® (Corning), and added to the μ-Dish center well. Following polymerization as described above, media was removed from all wells, and 3-well insert was carefully removed. The gap between wells was filled with additional Matrigel to form a continuous substrate and allow for cell migration, and allowed to polymerize for 20 minutes. Complete media was added to cover cells, and incubated at 37° C. in humidified conditions with 5% CO2. IF images were acquired using a Nikon A1RMP confocal microscope at 1- and 24-hours following co-culture, and T cell chemotaxis was quantified as the number of migratory cells visible in either the EGFP or CXCL9 (RFP+) transduced tumor/astrocyte field.

### Single Cell RNA Sequencing, Quality Control, and Data Analysis

Following whole brain resection, cerebellar tissue was removed and the right hemisphere collected for processing. Tissue dissociation and CD45 TIL isolation was performed as described under Tissue Dissociation above. The cells directly after depletion were washed with PBS and 0.04% bovine serum albumin two times and filtered with 40-un cell drainer. Cells were collected by centrifugation at 500 g for 5 min and subsequently counted with hemocytometer. Cells were diluted in ice-cold PBS containing 0.04% BSA at a density of 1000 cells/μL. The final cell suspension volume equivalent to 8000 target cells was used for further processing. Cells were loaded into a Chromium NextGEM Chip G (10× Genomics, Pleasanton, California) and processed in Chromium X following the manufacturer's instructions. Preparation of gel beads in emulsion and libraries were performed with Chromium Next GEM Single Cell 3′ Kit v.3.1 (Dual Index) according to User Guide provided by the manufacturer. Libraries quality and quantity were verified with 2200 TapeStation (Agilent technologies, USA). Libraries were pooled based on their molar concentrations. Pooled library was sequencing on the NovaSeq 6000 instrument (Illumina, San Diego, California). For sequencing 3′ gene expression libraries, the following read length was used: Read 1-28 cycles; i7Index-10 cycles; i5Index-10 cycles; Read 2-90 cycles. Raw base call (BCL) files generated by NovaSeq 6000 sequencer were processed using Cell Ranger software (10X Genomics, version 7) for demultiplexing, barcode processing, and single-cell 3′-gene counting. Mouse genome reference GRCm38 was used for sequence alignment using STAR aligner. A read was considered exonic, if at least 50% of it mapped to an exon, intronic (if it was non-exonic and intersected an intron), or intergenic otherwise. For reads that aligned to a single exonic locus but also aligned to 1 or more non-exonic loci, the exonic locus was prioritized and the read was considered to be confidently mapped to the exonic locus. Cell Ranger also aligned exonic reads to annotated transcripts. An annotated transcript that aligned to the same strand was considered to be confidently mapped to the transcriptome. These confidently mapped reads were used for unique molecular identifier (UMI) counting and subsequent analysis to generate h5 files. The h5 file of each sample was then processed with Partek Flow analysis software (version 10). Cells meeting the following quality control (QC) parameters were included in the analysis: total reads between 1000 to 33,649; expressed genes between 187 to 5464; mitochondrial reads percentage <20%. Following this selection, 48159 cells that passed QC filters were obtained. Next, features were filtered in order to include only genes expressed in more than 0.01% of cells and 20,785 genes were retained. UMI counts were then normalized following Partek® Flow® recommendations: for each UMI in each sample the number of raw reads was divided by the number of total mapped reads in that sample and multiplied by 1,000,000, obtaining a count per million value (CPM), the normalized expression value was log-transformed. Starting from the normalized data node, clustering analysis were performed for each sample separately by means of graph-based clustering task in Partek® Flow® software which employs the Louvain algorithm. Clustering analysis was done based on the first 100 principal components. To visualize single cells in a two-dimensional space, a Uniform Manifold Approximation and Projection (UMAP) plot was applied using the first 50 principal components for each sample separately and for the entire data set. Cell types were determined by the expression of marker genes that define specific cell types (Table 1). Pathway enrichment analysis for tumor cells and immune cells was performed with AUCell algorithm using the NanoString nCounter Immune Exhaustion panel. Interactions between immune populations were analyzed and visualized using the CellChat algorithm. The pheatmap package was used for unsupervised hierarchical clustering to create heatmaps (Kolde, R., Pheatmap: pretty heatmaps. R package version, 1 (2), 726.).

TABLE 1
Non-limiting gene markers for target groups of interest.

| Target group | Gene markers | | | Reference |
|---|---|---|---|---|
| canonical MG | Fcrls | Tmem119 | P2ry12 | Ochocka et al (32) |
| Disease associated MG | Lpl | Cst7 | | Ochocka et al (32) |
| Macrophages | Ly6c2 | Ccr2 | Ifitm2 | Ochocka et al (32) |
| Border-associated macrophages | Cd163 | Mrc1 | Lyve1 | Ochocka et al (32) |
| Macrophages suppresive | Ccl22 | Cd274 | | Ochocka et al (32) |
| Monocytes | Ly6c1 | | | Ochocka et al (32) |
| Monocytes non-classical | Spn | Cx3cr1 | Tnfrsf1b | Ochocka et al (32) |
| DC | Xcr1 | Flt3 | Cd24a | Hongo et al (30) |
| NK cells | Klra1 | Ncr1 | | Ochocka et al (32) |
| B cells | Cd79a | Cd79b | Ms4a1 | Lee et al. (29) |
| CD8+ T cells | Cd3d | Cd8a | Cd8b1 | Zheng et al (31) |
| CD4+ T cells | Cd3d | Cd4 | | Zheng et al (31) |
| Treg | Cd4 | Foxp3 | Il2ra | Zheng et al (31) |

### Statistical Analysis

Statistical analyses performed using GraphPad Prism 9 as described in figure legends. Significance determined as p<0.05. Cell counts on 3D IHC images were established using Imaris ×64 v9.7.0, where cell counts were based on target channel fluorescence from ‘spots’ identified as a diameter of >6 μm and a set minimum voxel intensity, with background subtraction applied. For survival studies, animals were randomized prior to treatment.

### Other Embodiments

It should be understood that the examples and embodiments described herein are for illustrative purposes only and that various modifications or changes in light thereof may be suggested to persons skilled in the art and are to be included within the spirit and purview of this application and the scope of the appended claims. Recitation of ranges of values herein are merely intended to serve as a shorthand method of referring individually to each separate value falling within the range, unless otherwise indicated herein, and each separate value is incorporated into the specification as if it were individually recited herein. The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein may be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are chemically and/or physiologically related may be substituted for the agents described herein while the same or similar results would be achieved. All such similar substitutes and modifications apparent to those skilled in the art are deemed to be within the spirit, scope and concept of the invention as defined by the appended claims.

1. A recombinant adeno-associated virus (rAAV) particle comprising:
a nucleic acid vector comprising an expression cassette comprising a transgene encoding a chemokine, wherein the chemokine is selected from CXCL9, MIP-1α/β, CXCL10, CXCL16, and variants thereof, wherein the transgene is operably linked to a promoter, and wherein the expression cassette is flanked on each side by an inverted terminal repeat sequence (ITR) of serotype 2; and
a capsid protein of serotype 6.

2-3. (canceled)

4. The rAAV particle of claim 1, wherein the promoter is a promoter that mediates expression in neurons and/or glial cells.

5. The rAAV particle of claim 1, wherein the promoter is selected from a Glial Fibrillary Acidic protein (GFAP) promoter, MECP2 promoter, IBA1 promoter, JeT promoter, human synapsin (hSYN) promoter, and Microtubule-associated protein 2 (MAP2) promoter.

6-7. (canceled)

8. The rAAV particle of claim 1, wherein the nucleic acid vector comprises a nucleic acid sequence having at least about 85% sequence identity to the sequence of SEQ ID NO: 5.

9. (canceled)

10. The rAAV particle of claim 1, wherein the rAAV particle is an rAAV2/6-GFAP-CXCL9 particle.

11. (canceled)

12. The rAAV particle of claim 1, wherein the transgene comprises a nucleic acid sequence that is at least about 90%, 92.5%, 95%, 98%, or 99% identical to SEQ ID NO: 3 or 4.

13-20. (canceled)

21. A kit comprising:
i) a first composition comprising the rAAV particle of claim 1; and
ii) a second composition comprising an immune checkpoint inhibitor.

22. The kit of claim 21, wherein the immune checkpoint inhibitor is a PD-1 antagonist.

23-25. (canceled)

26. The kit of claim 21, wherein the first composition is adapted for intracranial or ICV delivery, and/or the second composition is adapted for intravenous delivery.

27. A method of treating a subject suffering from a cancer, wherein the cancer is a solid tumor, comprising administering a therapeutically effective amount of the rAAV particle of claim 1.

28. The method of claim 27, wherein the rAAV particle is administered via an intracranial or ICV injection.

29. The method of claim 27, wherein the cancer is a brain tumor.

30. (canceled)

31. The method of claim 27, wherein the cancer is a non-brain solid tumor that has metastasized to the brain.

32. The method of claim 27, wherein the subject is a human.

33. The method of claim 27, wherein the rAAV particle or composition is administered directly into the tumor.

34. The method of claim 27, wherein the rAAV particle is administered in amount of between about 1×10⁹ and about 1×10¹¹ rAAV vector genomes.

35-41. (canceled)

42. The method of claim 27 further comprising administering a PD-1 antagonist.

43-46. (canceled)

47. A method of contacting a neuron, glial cell or astrocyte with the rAAV particle of claim 1.

48. (canceled)

49. A method of treating a subject suffering from glioma or GBM, the method comprising:
administering a therapeutically effective amount of an rAAV particle comprising (i) a nucleic acid vector comprising an expression cassette comprising a transgene encoding a chemokine, wherein the chemokine is human CXCL9 or a variant thereof, wherein the transgene is operably linked to a promoter, and wherein the expression cassette is flanked on each side by an ITR of serotype 2, and (ii) a capsid protein of serotype 6,
and wherein the administration results in expression of a therapeutically effective amount of human CXCL9, thereby treating the glioma or GBM.

50-51. (canceled)

52. The rAAV particle of claim 1, wherein the transgene comprises the nucleic acid sequence of SEQ ID NO: 3 or 4.


SEQUENCE LISTING (7 sequences)

SEQ ID NO: 1 - DNA, 21 bp, synthetic construct
  source 1..21: mol_type = other DNA
ggaacccctagtgatggagtt

SEQ ID NO: 2 - DNA, 16 bp, synthetic construct
  source 1..16: mol_type = other DNA
cggcctcagtgagcga

SEQ ID NO: 3 - DNA, 804 bp, Mus musculus
  source 1..804: mol_type = genomic DNA
gggcatatggcttcagacaccagaaggatacaagcagcagcgagtaccagtcccttttctgttctgctgacaagctcaccctctgtcacctgctcaacatcatgaaggtctccaccactgcccttgctgttcttctctgtaccatgacactctgcaaccaagtcttctcagcgccatatggagctgacaccccgactgcctgctgcttctcctacagccggaagattccacgccaattcatcgttgactattttgaaaccagcagcctttgctcccagccaggtgtcattttcctgactaagagaaaccggcagatctgcgctgactccaaagagacctgggtccaagaatacatcactgacctggaactgaatgcctgagagtcttggaggcagcgaggaaccccccaaacctccatgggtcccgtgtagagcaggggcttgagccccggaacattcctgccacctgcatagctccatctcctataagctgtttgctgccaagtagccacatcgagggactcttcacttgaaattttatttaatttaatcctattggtttaatactatttaattttgtaatttattttattgtcatacttgtatttgtgactatttattctgaaagacttcaggacacgttcctcaacccccatctccctcccagttggtcacactgtttggtgacagctattctaggtagacatgatgacaaagtcatgaactgacaaatgtacaatagatgctttgtttataccagagaagtaataaatatgccctttaacaagtgaaaaaaaaaaaaaaa

SEQ ID NO: 4 - DNA, 678 bp, Homo sapiens
  source 1..678: mol_type = genomic DNA
ctctttaagacttttatttttatctctagaaggggtcttagccccctagtctccagttgctgctgacacgccgaccgcctgctgcttcagctacacctcccggcagattccacagaatttcatagctgactactttgagacgagcagccagtgctccaagcccggtgtcatcttcctaaccaagcgaagccggcaggtctgtgctgaccccagtgaggagtgggtccagaaatatgtcagcgacctggagctgagtgcctgaggggtccagaagcttcgaggcccagcgacctcggtgggcccagtggggaggagcaggagcctgagccttgggaacatgcgtgtgacctccacagctacctcttctatggactggttgttgccaaacagccacactgtgggactcttcttaacttaaattttaatttatttatactatttagtttttgtaatttattttcgatttcacagtgtgtttgtgattgtttgctctgagagttcccctgtcccctcccccttccctcacaccgcgtctggtgacaaccgagtggctgtcatcagcctgtgtaggcagtcatggcaccaaagccaccagactgacaaatgtgtatcggatgcttttgttcagggctgtgatcggcctggggaaataataaagatgctcttttaaaaggtaaa

SEQ ID NO: 5 - DNA, 7212 bp, synthetic construct
  source 1..7212: mol_type = other DNA
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtaccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaagaattggatcaaagcttcaattggatatcactagcgctaccggactcagatctcgagctcaatgaagtccgctgttcttttccttttgggcatcatcttcctggagcagtgtggagttcgaggaaccctagtgataaggaatgcacgatgctcctgcatcagcaccagccgaggcacggtccactacaaatccctcaaagacctcaaacagtttgccccaagccccaattgcaacaaaactgaaatcattgctacactgaagaacggagatcaaacctgcctagatccggactcggcaaatgtgaagaagctgatgaaagaatgggaaaagaagatcaaccaaaagaaaaagcaaaagagggggaaaaaacatcaaaagaacatgaaaaacagaaaacccaaaacaccccaaagtcgtcgtcgttcaaggaagactacagattacaaggatgacgacgataagtaaagcttcgaattctgcagtcgacggtaccgcgggcccgggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgccctgtacaagtaaagcggccgcgactctagatcataatcagccataccacatttgtagaggttttacttgctttaaaaaacctcccacacctccccctgaacctgaaacataaaatgaatgcaattcctcgagcagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattc

SEQ ID NO: 6 - DNA, 21 bp, synthetic construct
  source 1..21: mol_type = other DNA
cactccctctctgcgcgctcg

SEQ ID NO: 7 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct